(19) United States
(12) Patent Application Publication
(10) Pub. No.: US 2007/0111247 A1
(43) Pub. Date: May 17, 2007

(54) Systems and methods for the biometric analysis of index founder populations

(76) Inventors: Joel C. Stephens, Guilford, CT (US); Joseph R. Flicek, New York, NY (US)

Correspondence Address: JONES DAY, 222 EAST 41ST ST, NEW YORK, NY, 10017, US

(21) Appl. No.: 11/601,471

(22) Filed: Nov. 17, 2006

Related U.S. Application Data
(60) Provisional application No. 60/737,900, filed on Nov. 17, 2005.

(51) Int. Cl. C12Q 1/68; G06F 19/00

(52) U.S. Cl. 435/6; 702/20

(57) ABSTRACT

Systems, methods and apparatus for associating a clinical parameter with one or more candidate chromosomal regions in the human genome are provided. An index founder population is identified in a test population based upon the genotype X of each member of the test population such that the posterior probability Pr(K|X) for the index founder population is greater for K=1 than any other integer K, where K is the number of subpopulations in the index founder population. The clinical parameter is measured for each respective member of the index founder population. Then a quantitative phenotypic analysis is performed between (i) the genotype X of each respective member of the index founder population and (ii) the clinical parameter thereby identifying one or more candidate chromosomal regions in the human genome that associate with the clinical parameter.

### CROSS REFERENCE TO RELATED APPLICATION

This application claims priority to U.S. Patent Application No. 60/737,900, filed Nov. 17, 2005, which is hereby incorporated by reference herein in its entirety.

### 1. FIELD OF THE INVENTION

The field of this invention relates to computer systems and methods for identifying genes and biological pathways associated with phenotypes within index founder populations.

2. BACKGROUND OF THE INVENTION

In the past decade, technical advances in the areas of DNA sequencing and data or information mining have led to the industrialization of the gene discovery process and the sequencing of the human genome. This sequence now provides a wealth of potential targets for the development of new therapeutics to treat human diseases. Proper use of new technology is now required to validate the roles that these genes play in human diseases and to discover new drugs at the scale and scope of the genome. With the elucidation of the sequence of the human genome, a complete list all human genes is rapidly being completed. Researchers now agree that there exists an unprecedented opportunity to understand the mechanistic basis of major human diseases and to develop novel therapeutics to improve human health.

Advances in molecular biology, genetics, and information technology over the past 25 years have led to the identification of many gene mutations that underlie inherited diseases. Included in this list are the CFTR gene in cystic fibrosis, the IT15 gene in Huntington's disease, the Bcr-Abl fusion gene in chronic myeloid leukemia, and the LDL receptor in familial hypercholesterolemia. The absolute correlation between the presence of these genetic variants and disease pathology has provided support for the molecular basis of disease and resulted in a major shift in drug discovery efforts in the pharmaceutical industry from activity-based screens to molecular target-based approaches.

Linkage and association analyses in humans has been performed successfully for fine mapping of a large number of genes that have large effect on rare phenotypes that segregate in pedigrees.

There are a large number of complex diseases that are far more common, yet tend to occur more frequently among relatives of affected individuals than in the general population and have substantial heritability. In most cases of complex diseases, a single gene of small effect is not sufficient to produce a clinical symptom, but the combined effect of multiple genes confers additive genetic contributions.

Because there is a clear genetic component to these diseases, it is believed that allelic association and linkage analysis methods could identify the genes underlying these complex traits. The difficulty is that the effect of any single allele on the risk for chronic disease is typically weak and therefore more difficult to identify. Thus, what is needed in the art are systems and methods for making this statistical pattern identification problem more tractable.

### 3. SUMMARY OF THE INVENTION

One aspect of the invention provides a method of associating a clinical parameter with one or more candidate chromosomal regions in the human genome. In the method, a test population is validated as an index founder population. To accomplish this, the genotype X of each member of the test population is received or obtained (e.g., by clinical measurement). When the posterior probability Pr(K|X) for the test population is greatest for K=1, where K represents the number of subpopulations in the test population, the test population is deemed to be an index founder population. In the method, the clinical parameter is measured from each respective member of the index founder population. Then a quantitative trait locus analysis is performed between (i) the genotype X of each member of the index founder population and (ii) the clinical parameter thereby identifying one or more candidate chromosomal regions in the human genome that associate with the clinical parameter.

In some embodiments, the quantitative trait locus analysis comprises (i) testing for linkage between a position in a chromosome, in the human genome, and the clinical parameter used in the quantitative trait locus analysis; (ii) advancing the position in the chromosome by an amount; and (iii) repeating steps (i) and (ii) until an end of the chromosome is reached. In some embodiments, steps (i) through (iii) are repeated for each chromosome in the human genome. In some embodiments, the amount that is advance is less than 100 centiMorgans, less than 10 centiMorgans, less than 5 centiMorgans, or less than 2.5 centiMorgans. In some embodiments, the testing comprises performing linkage analysis or association analysis. In some embodiments, the quantitative trait locus analysis computes a logarithm of the odds score at each position.

In some embodiments, the genotype comprises at least five markers, at least one hundred markers, at least one thousand markers, or at least twenty thousand markers. In some embodiments, the clinical parameter is an abundance level measurement associated with a gene in a biological sample obtained from the respective member. In some embodiments, the abundance level measurement associated with the gene is determined by measuring an amount of a cellular constituent encoded by the gene in one or more cells in the biological sample. In some embodiments, the amount of the cellular constituent comprises an abundance of an RNA species present in or secreted by one or more cells in the biological sample. In some embodiments, the abundance of the RNA species is measured by contacting a gene transcript array with an RNA species from the one or more cells, or with nucleic acid derived from the RNA species, where the gene transcript array comprises a positionally addressable surface with attached nucleic acids or nucleic acid mimics, the nucleic acids or nucleic acid mimics capable of hybridizing with the RNA species, or with nucleic acid derived from the RNA species. In some embodiments, the amount of the cellular constituent comprises an abundance of a protein present in or secreted by one or more cells in the biological sample.

In some embodiments, the clinical parameter is a heart rate, a skin reflectivity, a blood pressure, a cholesterol level, or a tryglyceride level. In some embodiments, the clinical parameter is absence, presence, or stage of a disease. In some embodiments, the disease is a complex disease such as adult macular degeneration, asthma, ataxia telangiectasia, autism, bipolar disorder, breast cancer, a cancer, cardiomyopathy, celiac disease, Charcot-Marie-Tooth disease, colon cancer, insulin-dependent diabetes mellitus, T2 diabetes, diabetic retinopathy, dementias, early-onset Parkinson's disease, epilepsy, familial hypercholesteremia, glaucoma, heart disease, hereditary early-onset Alzheimer's disease, hereditary nonpolyposis, hypertension, infection, late-onset Alzheimer's disease, late-onset Parkinson's disease, leukemias, longevity, lung cancer, maturity-onset diabetes of the young, mellitus, migraine, myofibrillar myopathy, multiple sclerosis, a neuropathy, nonalcoholic fatty liver (NAFL), nonalcoholic steatohepatitis (NASH), non-insulin-dependent diabetes mellitus (NIDDM), non-syndromic blindness, non-syndromic deafness, neuropathies, osteoporosis, pancreatic diabetes, pancreatic cancer, parkinsonisms, polycystic kidney disease, prostate cancer, psoriases, rheumatoid arthritis, schizophrenia, sickle cell disease, steatohepatitis, stroke, systemic lupus erythematosus, or xeroderma pigmentosum.

In some embodiments, the clinical parameter is body temperature, respiration rate, pulse, blood pressure, or tryglyceride level. In some embodiments, the posterior probability Pr(K|X) for the index founder population for any K less than 6 and greater than 1 is 0.4 or less. In some embodiments, the posterior probability Pr(K|X) for the index founder population for any K less than 6 and greater than 1 is 0.3 or less.

In some embodiments, the method further comprises identifying each member of the index founder population using at least one criterion selected from the group consisting of geographical region, consanguinity, average family size, availability of medical records, and life expectancy. In some embodiments, the method further comprises obtaining a biological sample from each member of the test population, prior to the identifying step; and determining, for each respective member i of the test population, a genotype Xᵢ from the biological sample obtained from the respective member of the test population, prior to the identifying step. In some embodiments, the test population comprises more than 5 members, more than 10 members, more than 100 members, or more than 500 members. In some embodiments, the index founder population comprises less than 500 members. In some embodiments, the test population comprises more than 1000 members and the index founder population comprises less than 1000 members. In some embodiments, the test population comprises more than 2500 members and the index founder population comprises less than 2500 members.

Another aspect of the present invention provides a computer program product for use in conjunction with a computer system. The computer program product comprising a user readable storage medium and a computer program mechanism embedded therein. The computer program mechanism is for associating a clinical parameter with one or more candidate chromosomal regions in the human genome. The computer program mechanism comprises instructions for identifying an index founder population in a test population based upon the genotype X of each member of the test population, the posterior probability Pr(K|X) for the index founder population is greatest for K=1, and where K represents the number of subpopulations in the index founder population. The computer program mechanism further comprises instructions for receiving a measurement of the clinical parameter from each respective member of the index founder population; and instructions for performing a quantitative trait locus analysis between (i) the genotype X of each member of the index founder population and (ii) the clinical parameter thereby identifying one or more candidate chromosomal regions in the human genome that associate with the clinical parameter.

Still another aspect of the present invention provides a computer system for associating a clinical parameter with one or more candidate chromosomal regions in the human genome. The computer system comprises a processor and a memory encoding one or more programs coupled to the processor. The one or more programs cause the processor to perform a method comprising:

(i) identifying an index founder population in a test population based upon the genotype X of each member of the test population, where the posterior probability Pr(K|X) for the index founder population is greatest for K=1, and where K represents the number of subpopulations in the index founder population;

(ii) receiving a measurement of the clinical parameter from each respective member of the index founder population; and

(iii) performing a quantitative trait locus analysis between (i) the genotype X of each member of the index founder population and (ii) the clinical parameter thereby identifying one or more candidate chromosomal regions in the human genome that associate with the clinical parameter.

### 4. BRIEF DESCRIPTION OF THE DRAWINGS

FIG. 1 illustrates a computer system for associating a clinical parameter with one or more candidate chromosomal regions in the genome of a human in accordance with one embodiment of the present invention.

FIG. 2 illustrates a method for associating a clinical parameter with one or more candidate chromosomal regions in the genome of a human in accordance with one embodiment of the present invention.

FIG. 3 illustrates an exemplary expression statistic set in accordance with one embodiment of the present invention.

FIG. 4 illustrates the Gulf States in their regional settings.

FIG. 5 illustrates an enlarged view of the Gulf States.

Like reference numerals refer to corresponding parts throughout the several views of the drawings.

### 5. DETAILED DESCRIPTION

5.1 Definitions

As used herein, the terms “disease” and “disorder” are used interchangeably to refer to a condition in a subject. Preferably, the condition is a pathological condition.

As used herein, the terms “gene expression” and “expression of a gene” refer to gene expression detected and/or measured at either the RNA or protein level, or both. In certain embodiments, either total RNA or mRNA is detected and/or measured. It is appreciated that mRNA may be detected and/or measured indirectly, for example by the detection of cDNA. In certain embodiments, RNA, mRNA, or cDNA is detected and/or measured, for example, via hybridization assays or PCR-based assays. In other embodiments, protein is detected and/or measured, for example, via immunoassays, or assays for protein activity. In still other embodiments, mRNA and protein are both detected and/or measured.

As used herein, the terms “peptide, polypeptide, and protein” are used to refer to amino acid sequences of various approximate lengths. For example, a peptide refers to a chain of two or more amino acids joined by peptide bonds, generally of less than about 50 amino acid residues, while a polypeptide refers to a longer chain of amino acids. In the context of a polypeptide that is a portion of a protein, the polypeptide is a chain of amino acids that is less in length than the length of the protein. It is appreciated that the terms “peptide” and “polypeptide” are not meant to refer to a precise length of a chain of amino acid residues and that in certain contexts, the two terms may be used interchangeably.

As used herein, the terms “subject”, “patient” and “member” are used interchangeably to refer to a human subject.

As used herein, the term “subpopulation” refers to a population, which optionally a subset of a larger population, that is deemed to have a common ancestral origin. The subpopulation may be any portion of the larger population, including the entirety of the larger population, so long as the subpopulation is deemed to have a common ancestral origin. In other words, a subpopulation does not exhibit genetic admixture with respect to specific markers that are used to distinguish whether subjects have a common ancestral origin. See, for example, Gower, 2003, Diabetes 52, 1047, which is hereby incorporated by reference, for an exemplary implementation of genetic admixture analysis.

As used herein, the terms “therapy” and “therapeutic” refers to any protocol, method and/or agent that can be used in the prevention, treatment, management or amelioration of a disorder or one or more symptoms thereof. In certain embodiments, the terms “therapies” and “therapy” refer to a biological therapy, supportive therapy, and/or other therapies useful in treatment, management, prevention, or amelioration of a disorder or one or more symptoms thereof known to one of skill in the art such as medical personnel.

5.2 Exemplary System and Method

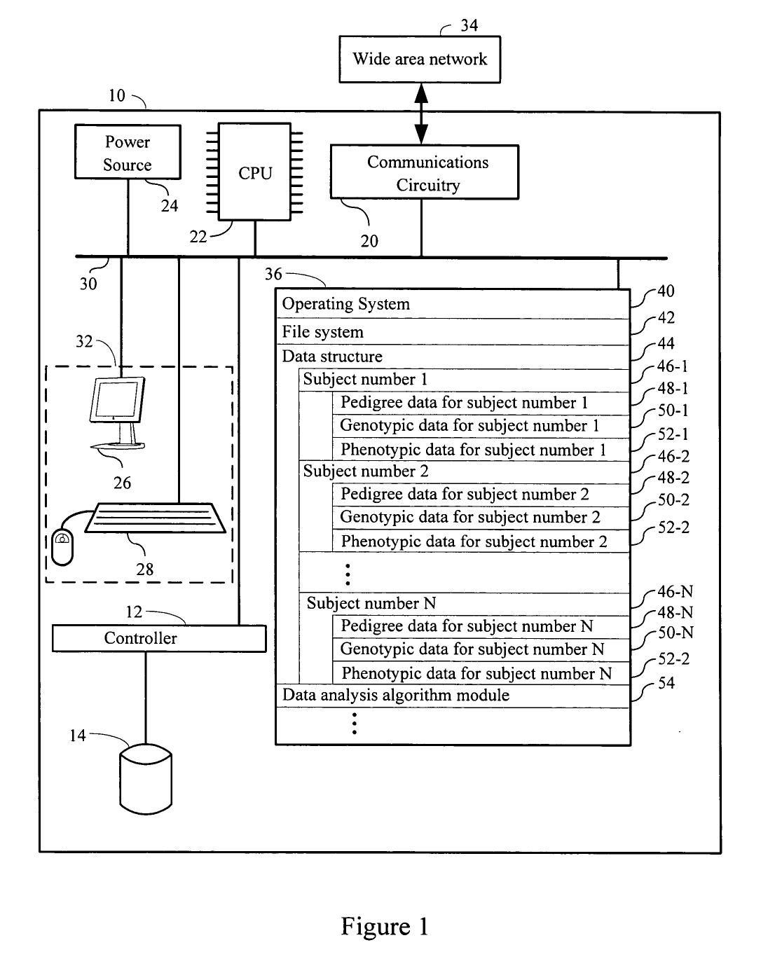

It is widely acknowledged that data about the level and nature of linkage disequilibrium between alleles of tightly linked single nucleotide polymorphisms (SNPs) can be readily found. Increasing evidence of allelic heterogeneity at the loci predisposing to complex disease has been observed. The present invention provides improved systems and methods for performing this form of analysis. Index founder populations are selected in the present invention that improve the probability of identifying the true or most significant genes or family of interacting genes. The present invention provides methods, computer systems, and computer program products for performing such selections and genetic analysis. FIG. 1 details an exemplary computer system in accordance with one such embodiment of the present invention.

The computer system of FIG. 1 is preferably a computer system 10 having:

a central processing unit 22;

a main non-volatile storage unit 14, for example, a hard disk drive, for storing software and data, the storage unit 14 controlled by storage controller 12;

a system memory 36, preferably high speed random-access memory (RAM), for storing system control programs, data, and application programs, comprising programs and data loaded from non-volatile storage unit 14; system memory 36 may also include read-only memory (ROM);

a user interface 32, comprising one or more input devices (e.g., keyboard 28) and a display 26 or other output device;

a network interface card 20 for connecting to any wired or wireless communication network 34 (e.g., a wide area network such as the Internet);

an internal bus 30 for interconnecting the aforementioned elements of the system; and

a power source 24 to power the aforementioned elements.

Operation of computer 10 is controlled primarily by operating system 40, which is executed by central processing unit 22. Operating system 40 can be stored in system memory 36. In addition to operating system 40, in a typical implementation system memory 36 includes:

file system 42 for controlling access to the various files and data structures used by the present invention;

a data structure 44 for storing biological information about an index founder population in accordance with the present invention; and

a data analysis algorithm module 54 for associating traits with genetic loci in accordance with the present invention.

As illustrated in FIG. 1, computer 10 comprises software program modules and data structures. Each of the data structures can comprise any form of data storage system including, but not limited to, a flat ASCII or binary file, an Excel spreadsheet, a relational database (SQL), or an on-line analytical processing (OLAP) database (MDX and/or variants thereof). In some specific embodiments, such data structures are each in the form of one or more databases that include hierarchical structure (e.g., a star schema). In some embodiments, such data structures are each in the form of databases that do not have explicit hierarchy (e.g., dimension tables that are not hierarchically arranged).

In some embodiments, each of the data structures stored or accessible to system 10 are single data structures. In other embodiments, such data structures in fact comprise a plurality of data structures (e.g., databases, files, archives) that may or may not all be hosted by the same computer 10. For example, in some embodiments, data structure 44 comprises a plurality of Excel spreadsheets that are stored either on computer 10 and/or on computers that are addressable by computer 10 across wide area network 34. In another example, data structure 44 comprises a database that is either stored on computer 10 or is distributed across one or more computers that are addressable by computer 10 across wide area network 34.

It will be appreciated that many of the modules and data structures illustrated in FIG. 1 can be located on one or more remote computers. For example, some embodiments of the present application are web service-type implementations. In such embodiments, a data analysis algorithm module 54 and/or other modules can reside on a client computer that is in communication with computer 10 via network 34. In some embodiments, for example, a data analysis algorithm module 54 can be an interactive web page.

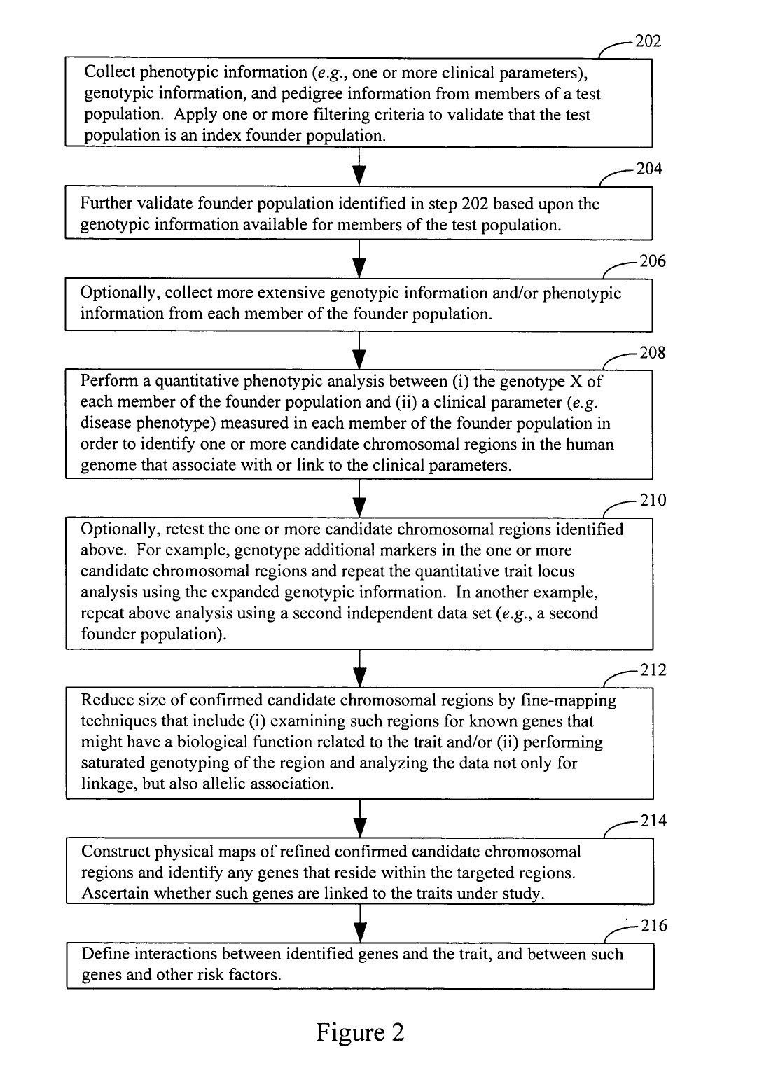

Now that an exemplary computer system has been described, one novel method that is performed in accordance with the systems and methods of the present invention will be described in conjunction with FIG. 2. Such systems and methods can be used to identify index founder population that can be used to identify genes or proteins that link to diseases. Exemplary populations that can be used to elucidate indexed founder populations using the systems and methods of the present invention are described in Section 5.3. Exemplary diseases that can be elucidated using the systems and methods of the present invention are described in Section 5.12.

In the following steps, a potential index founder population is selected. Then, the systems and methods of the present invention apply one or more filtering criteria to validate that the potential index founder population is an index founder population.

Step 202. In step 202, phenotypic information (e.g, disease phenotype, one or more clinical parameters, etc.), genotypic information, and pedigree data from members of a test population (potential index founder population) is collected. In some embodiments, the phenotypic information is stored as data 52, the genotypic information is stored as data 50, and the pedigree data is stored as data 48 in data structure 44 in computer system 10. In some embodiments, the test population comprises more than 5 members, more than 500 members, more than 1000 members, or more than 2500 members.

In typical embodiments, phenotypic information is collected for all or a portion of the members of the test population. In some embodiments, a “portion of the members of the test population” is at least X % of the test population, where X=10, 25, 50, 60, 70, 80, 90, or 95. Exemplary phenotypic information (e.g., clinical parameters, disease phenotype) that can be measured in a population and stored as phenotypic data 52 in data structure 44 of computer system 10 include, but are not limited to, age, body mass index (BMI), diastolic blood pressure, diet, electrocardiogram, environmental exposure, ethnicity, exercise logs, heart rate, height, gender, glycaemic parameters, glucose levels, hematocrit, insulin resistance index, lipid profile, medical disorders, medication, mental disorder, physical activity, serum adiponectin levels, smoking habits, systolic blood pressure, triglyceride levels, uric acid, weight, absence/presence of disease, and disease stage. In some embodiments of the present invention, candidate subjects 46 provide answers to questionnaires designed to elicit information relating to one or more of the factors that define an index founder population.

In typical embodiments, pedigree data is collected for all or a portion of the members of the test population. In some embodiments, a “portion of the members of the test population” is at least X % of the test population, where X=10, 25, 50, 60, 70, 80, 90, or 95. In one embodiment, the pedigree data comprises, for each member of the test population from which pedigree data is obtained, any combination of (i) a pedigree number, (ii) an individual identification number, (iii) a father's identification number, (iv) a mother's identification number, (v) a first offspring identification number, (vi) a next paternal sibling identification number, (vii) a next maternal sibling identification number, (viii) sex, and (ix) a proband status. A proband is the first affected individual in a family with a genetic disorder who is manifesting the disease and is diagnosed so. Between the ancestors of the proband, there are other members with the manifest disease, but they might be unknown due to the lack of information regarding those individuals or the disease at the time they lived. Other ancestors might be undiagnosed due to the incomplete penetration or variable expression. The diagnosis of the proband raises the level of suspicion for the proband's relatives and some of them may be diagnosed with the same disease. Conventionally, when drawing a pedigree chart, instead of the first diagnosed person, the proband may be chosen between the manifestly ill ancestors (parents, grandparents) from the first generation where the disease is found.

In typical embodiments, genotypic data is collected for all or a portion of the members of the test population. In some embodiments, a “portion of the members of the test population” is at least X % of the test population, where X=10, 25, 50, 60, 70, 80, 90, or 95. Such genotypic data can be collected using, for example, the methods described in Section 5.4, below.

In some embodiments, test populations are selected from distinct geographical sources so that genetic variability is minimized. Examples of geographic regions having populations with reduced genetic variability include, but are not limited to, Kuwait, the United Arab Emirates, Qatar, Yemen, Saudi Arabia, Oman, and India as described in Section 5.3, below. However, the present invention in not limited to such embodiments. In some embodiments, populations that have reduced genetic variability but are not restricted to a specific geographical location (e.g., some nomadic populations) are sought. In general, what is sought are populations that have reduced genetic variability. Thus, for example, some nomadic populations that have a degree of genetic isolation are also used in some embodiments of the present invention.

Filtering criteria or factors are imposed in order to identify populations that are index founder populations. It is most likely that index founder populations will have reduced genetic variability. The filtering criteria serve to define index founder populations. The most important filtering criteria is consanguinity, which is described in further detail below. Additional, optional factors that can be used to help identify a population with reduced genetic variability include, but are not limited to, availability of medical records, degree of consanguinity (as a result of caste systems, political considerations, etc.), average family size, number of generations in the region, accessibility/willingness of the population, genetic isolation of the population, availability of historical population and demographic data, family structure (e.g., polygamous, monogamous), life expectancy, and whether population is nomadic or stationary agricultural based society.

In some embodiments of the present invention, candidate subjects provide answers to questionnaires designed to elicit information relating to one or more of the factors that define an index founder population. In some embodiments, factors are ranked on a scale that ranges from a first extreme indicating a high degree of suitability for defining an index founder population to a second extreme indicating a low degree of suitability for defining an index founder population. By way of a non-limiting illustration, each factor or criterion used to define an index founder population can be assigned a rank between 1 and 5, where 1 indicates poor suitability and 5 indicates high suitability. For example, consider the criterion average family size. In some embodiments, an average family size of 1, meaning that each family has only one child, may receive a value of 1 (poor suitability), whereas an average family size of 5, meaning that each family has five children, may receive a value of 5 (high suitability). In some embodiments, an index founder population is constructed from those subjects whose aggregate score is above a threshold value. Such an aggregate score is the arithmetic combination of the individual scores for each of the factors relevant to the index founder population. In some embodiments, degree of consanguinity is used to determine whether a population is a suitable index founder population. In some embodiments, degree of consanguinity and one or more of the above-identified factors are used to determine whether a population is a suitable index founder population. More description of factors that can be used to define an index founder population are set forth in Section 5.3.2, below. Information on how such factors can be used in an indexing system to screen a test population for a suitable index founder population is provided in Section 5.3.3 below, in particular, Section 5.3.3.1. In some embodiments, some factors have more weight than others in defining an index founder population. Accordingly, in some embodiments, the indexing scheme used to define an index founder population uses a weighting scheme such as any of those described in Section 5.3.3.2 below.

Step 204. The questionnaire based approach to defining an index founder population based on phenotypic information helps to identify suitable populations in accordance with the present invention. It will be appreciated that other methods besides questionnaires can be used. For example, relevant information may already be available in the form of demographic records, medical records, or other publicly accessible information.

In preferred embodiments, confirmation that index founder populations identified in any manner disclosed in step 202 above are in fact single populations as opposed to an admixture of two or more populations is sought. In some embodiments, such confirmation is sought by obtaining genotypic information from candidate subjects using the techniques disclosed in Section 5.4 below. Such genotypic information is then used in a confirmatory scoring scheme based on genotypes that are designed to determine whether the identified index founder population is truly a single population as opposed to an admixture of multiple populations. For example, in some embodiments, an index founder population is identified in a test population based upon the genotype X of each member of the test population, where the posterior probability Pr(K|X) for the index founder population is greatest for K=1, and where K represents the number of subpopulations in the index founder population. Examples of such confirmatory scoring schemes based on genotypic data (e.g., posterior probability Pr(K|X)), are described in Section 5.3.3.3. In some embodiments a population is referred to as a “test population” until it has been verified to a founder population using the systems and methods of the present invention.

Step 206. In some embodiments, an inexpensive initial genotypic screening test is performed on members of a test population in order to identify an index founder population. In some embodiments, once a potential index founder population is defined, more extensive genotypic information is obtained from the members of the index founder population using the techniques described, for example, in Section 5.4. In this second round of genotyping more extensive genotypic data is sought for a confirmatory scoring scheme based on genotypes such as the one disclosed in Section 5.3.3.3. Step 206 serves to remove subjects in the index founder population that do not belong to a single population, as determined by genetic criteria, and/or to rejection of a particular population outright because it represents more than one genetic population. In some embodiments, admixed individuals are excluded from the index founder population. In some embodiments, sequencing is done in addition to or instead of genotyping. Exemplary sequencing techniques are described in Section 5.14, below.

Step 208. One of the advantages of the index founder populations identified using the methods of the present invention is that smaller populations can be studied in follow up genetic studies as compared to instances where conventional outbred populations are studied. Accordingly, once an index founder population has been identified, quantitative phenotype analyses are performed using the genotypic data available for members of the index founder population and at least one clinical parameter measured for each member of the index founder population in order to identify one or more candidate chromosomal regions in the human genome that associate with (e.g., link to) the clinical parameters. In some embodiments, pathways can be identified using the methods disclosed in step 208.

For embodiments in which multiple tissue samples are collected from each member of the index founder population, a separate quantitative phenotype analysis can be performed for each different tissue sample. For example, in embodiments in which samples are collected from two different tissues, two different quantitative phenotype analyses are performed for each subject in the index founder population. In one embodiment, each quantitative phenotype analysis is performed by data analysis algorithm module 54 (FIG. 1). In one example, each quantitative phenotype analysis steps through each chromosome in the human genome. At each such location, a comparison is made between the genotype of one or more markers and the variation in the quantitative phenotype across the index founder population. Linkages, associations or other forms of genetic locus analysis are tested at each step or location along the length of the chromosome. In such embodiments, each step or location along the length of the chromosome can be at intervals that have an average length. In some embodiments, these regularly defined intervals are defined in Morgans or, more typically, centiMorgans (cM). A Morgan is a unit that expresses the genetic distance between markers on a chromosome. A Morgan is defined as the distance on a chromosome in which one recombinational event is expected to occur per gamete per generation. In some embodiments, each regularly defined interval is less than 100 cM. In other embodiments, each regularly defined interval is less than 10 cM, less than 5 cM, or less than 2.5 cM.

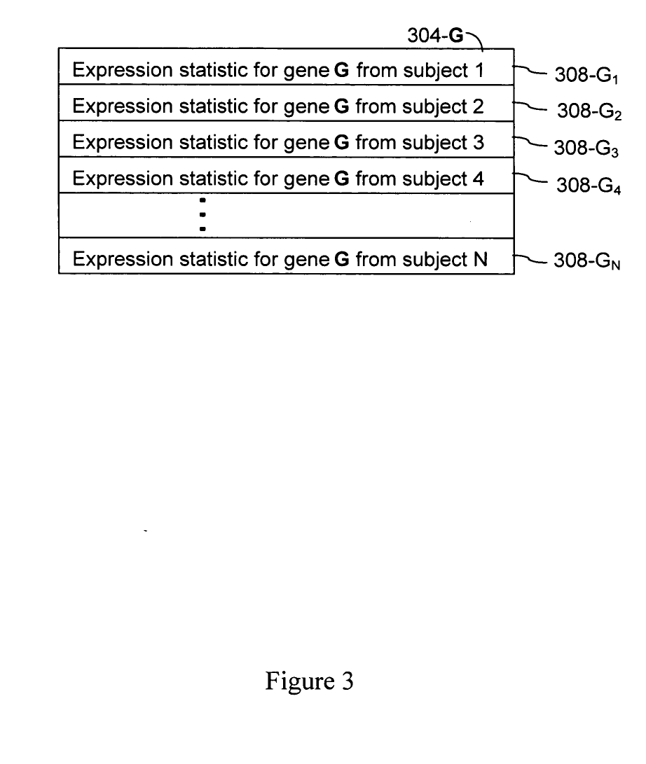

In each quantitative phenotype analysis, data corresponding to the measured clinical parameter under study is used as a disease phenotype. More specifically, for any given clinical parameter, the disease phenotype used in the quantitative phenotype analysis is the value for the clinical parameter from each member of the index founder population. In some embodiments, the clinical parameter is the expression of a gene. In such embodiments, an expression statistic set 304 is used as the quantitative trait, where the expression statistic set 304 comprises the corresponding expression statistic 308 for the gene 302 from all or a portion of the humans 306 in the index founder population under study. FIG. 3 illustrates an exemplary expression statistic set 304 in accordance with one embodiment of the present invention. Exemplary expression statistic set 304 includes the expression level 308 of a gene G (or cellular constituent that corresponds to gene G) from each member of the index founder population, including cases and controls. For example, consider the case where there are ten members in the index founder population, and each of the ten members expresses gene G. In this case, expression statistic set 304 includes ten entries, each entry corresponding to a different one of the ten humans in the plurality of humans. Further, each entry represents the expression level of gene G (or a cellular constituent corresponding to gene G) in the human represented by the entry. So, entry “1” (308-G-1) corresponds to the expression level of gene G (or a cellular constituent originating from the transcription or translation of gene G) in human 1, entry “2” (308-G-2) corresponds to the expression level of gene G (or a cellular constituent originating from the transcription or translation of gene G) in human 2, and so forth.

In one embodiment of the present invention, each quantitative phenotype analysis comprises: (i) testing for linkage or association between a position in a chromosome and the disease phenotype (e.g., expression values for a particular gene in each human in a plurality of humans) used in the quantitative phenotype analysis, (ii) advancing the position in the chromosome by an amount, and (iii) repeating steps (i) and (ii) until the end of the chromosome is reached. In some embodiments, the disease phenotype is an expression statistic set 304, such as the set illustrated in FIG. 3. More typically, the disease phenotype is another type of phenotypic characteristic, such as heart rate, a skin reflectivity, a blood pressure, a cholesterol level, or a tryglyceride level. In some embodiments, testing for linkage or association between a given position in the chromosome and the disease phenotype comprises correlating differences in the disease phenotype across the index founder population with differences in the genotype at the given position using a single marker test. Examples of single marker tests include, but are not limited to, t-tests, analysis of variance, or simple linear regression statistics. See, e.g., Statistical Methods, Snedecor and Cochran, 1985, Iowa State University Press, Ames, Iowa. However, there are many other methods for testing for linkage or association between a disease phenotype and a given position in the chromosome. In particular, if the disease phenotype is treated as the phenotype (in this case, a quantitative phenotype), then methods such as those disclosed in Doerge, 2002, Mapping and analysis of quantitative trait loci in experimental populations, Nature Reviews: Genetics 3:43-62, hereby incorporated herein by reference, may be used. Concerning steps (i) through (iii) above, if the genetic length of a given chromosome is N cM and 1 cM steps are used, then N different tests for linkage are performed on the given chromosome. This process can be repeated for each chromosome in the human genome.

In some embodiments, the data produced from each respective quantitative phenotype analysis comprises a logarithm of the odds score (LOD) computed at each position tested in the genome under study. A LOD score is a statistical estimate of whether two loci are likely to lie near each other on a chromosome and are therefore likely to be genetically linked. In the present case, a LOD score is a statistical estimate of whether a given position in the genome under study is linked to the disease phenotype corresponding to a given gene. LOD scores are further defined in Section 5.9, below. Generally, a LOD score of three or more suggests that two loci are genetically linked, a LOD score of four or more is strong evidence that two loci are genetically linked, and a LOD score of five or more is very strong evidence that two loci are genetically linked. However, the significance of any given LOD score may vary depending on the model used.

In some embodiments processing step 208 is essentially a linkage analysis, as described in Section 5.6, below. In other embodiments, processing step 208 is an allelic association analysis, as described in Section 5.7, below. In one form of association analysis, an affected population is compared to a control population. In particular, haplotype or allelic frequencies in the affected population are compared to haplotype or allelic frequencies in a control population in order to determine whether particular haplotypes or alleles occur at significantly higher frequency amongst affected samples compared with control samples. Statistical tests such as a chi-square test are used to determine whether there are differences in allele or genotype distributions.

Step 210. Step 208 serves to identify one or more candidate chromosomal regions. In some embodiments, verification that such regions link with clinical parameters associated with a disease is sought. In some embodiments, such verification is performed by retesting the linkage or association between the candidate chromosomal regions and a disease phenotype using an expanded set of genotypic markers from the candidate chromosomal regions. This may require expanded genotyping using, for example, the techniques disclosed in Section 5.4.2, below. In some embodiments, additional markers are genotyped in the one or more candidate chromosomal regions and the quantitative phenotypic analysis described in step 208 is repeated with the expanded genotypic information. In another example, steps 202 through 208 are repeated using a second independent data set. This second independent data set may be a second index founder population. In some instances, the second index founder population is constructed using the same factors and indexing scheme that was used to construct the original index founder population. In other instances, the second index founder population is constructed using different factors, different weights for such factors, and/or a different indexing scheme than was used for the original index founder population.

Step 212. In embodiments where, for example, the quantitative phenotypic analysis is linkage analysis, it is typically necessary to perform additional studies in order to reduce the size of the confirmed candidate chromosomal regions. For instance, a linkage analysis may produce a linkage region that spans a megabase of nucleotides or more. In fact, this linkage region may span dozens of genes. Thus, techniques are needed to pinpoint exactly what genetic variation within the linkage region is giving rise to a linkage with the disease phenotype. Methods by which this can be accomplished include, but are not limited to, fine-mapping techniques. Exemplary fine-mapping technique include: (i) examining such regions for known genes that might have a biological function related to the disease phenotype and/or (ii) performing saturated genotyping of the region and analyzing the data not only for linkage, but also allelic association. More details on suitable fine-mapping techniques are disclosed in Section 5.8, below.

In some embodiments, the candidate chromosomal regions are reduced by repeating the previous steps for a second index founder population. Phenotypic information (e.g., disease phenotype, one or more clinical parameters, etc.), genotypic information, and pedigree data from members of another test population are collected. In some embodiments, the new (second) test population belongs to a different race than the original (first) test population. In some embodiments, the new test population is the same race as the original test population. The filters described above are performed in order to verify that the new (second) test population in fact is a new (second) index founder population. Then, one or more candidate chromosomal regions are identified in the second index founder population using the same tests describe above. A composite genetic locus genetic locus that is linked or associated with a clinical parameter is taken as the intersection of the one or more chromosomal regions found in the first index founder population and the chromosomal regions found in the second index founder population. For example, consider the case in which the chromosomal regions A, B, and C are linked or associated with the disease phenotype in the first index founder population but only the chromosomal regions A and C are linked or associated with the disease phenotype in the second index founder population. In this instance, the intersection of the chromosomal regions found in the first index founder population and the chromosomal regions found in the second index founder population would consist of genomic regions A and C.

The size of the genetic loci (chromosomal regions) identified in the above-described techniques is dependent upon whether association analysis or linkage analysis is used to identify such genomic regions, the density of markers used in the analysis, as well as other factors. In some embodiments, each genetic locus (chromosomal regions) has a size of 10 megabases or less, 5 megabases or less, 1 megabase or less, between 50 kilobases and 5 megabases, or greater than 1 megabase.

Step 214. In step 214, a physical map of refined confirmed candidate chromosomal regions is constructed in order to identify any genes that reside within the targeted regions. Details on suitable techniques for identifying genes are disclosed in Section 5.9, below. When such genes are identified, the techniques disclosed in Sections 5.6 or 5.7 can be used to ascertain which of such genes are linked to the clinical traits under study. One of the advantage of the present invention is that necessity and sufficiency genes can be discovered in index founder populations that would not otherwise be discovered using conventional genetic techniques in the general population (e.g., a population that is not an index founder population). Necessity and sufficiency genes are described in Section 5.16, below. Thus, in some embodiments, the one or more chromosomal regions identified in the preceding steps encompasses a dominant or recessive necessity gene. In some embodiments, the one or more chromosomal regions identified in the preceding steps encompasses a dominant or recessive sufficiency gene.

Step 216. Once genes that link to the clinical traits under study are identified, the interactions that such genes make with other genes and other risk factors can be studied using known genetic techniques. Genes identified can be used for purposes described in Section 5.10. One such genetic technique is multivariate statistical methods such as those described in Section 5.13, below.

5.3 Index Founder Population

One of the advantages of the present invention is the elucidation of index founder populations as described in steps 202 and 204 of Section 5.2. Isolated populations are important in the discovery of disease genes for rare, single gene (Mendelian) disorders as well as common, polygenic (complex) diseases. Genetic isolates arise from a limited number of founders and can exist in cultural isolation within a specific geographic location (Arcos-Burgos and Muenke, 2002, Clin Genet. 61(4): 233-47). In nomadic situations, however, populations such as the Bedouins or Roma gypsies move from location to location but are still considered genetic isolates since they, like the stationary index founder populations, tend to practice endogamy (Farrer et al., 2003, J. Mol. Neurosci. 20(3): 207-12, Kalaydjieva et al., 2005, Bioessays 27: 1084-94). This prevents admixture with other genetic subgroups thus sustaining a homogenous index founder population. Marriage between closely related individuals further restricts genetic diversity within an index founder population, but most importantly, close-kin unions greatly influence the frequency of both benign and pathogenic gene variants. The presence of consanguinity in a population is an important determinant for the index founder population of the present invention and distinguishes it from classical genetic isolates such as Icelandic populations and Finish populations.

In some embodiments elucidation of an index founder population begins with the selection of subjects that reside or originate in specific geographic regions where populations have resided for relatively long periods of time with some degree of genetic isolation. Exemplary populations, organized by country of origin, are described Section 5.3.1, below. In some embodiments candidate populations that are not tied to a specific geographical location but nevertheless have reduced genetic variability (e.g., nomadic populations) are selected. Once a test population has been identified, additional filtering criteria, known as factors, may be applied in order to further define an index founder population. Exemplary filtering criteria are described in Section 5.3.2, below. Methods for applying such filtering criteria are described in Section 5.3.3, below. Of the factors, consanguinity is one of the most important. In some embodiments, an index founder population is one that is genetically isolated and in which there exists consanguinity.

5.3.1 Exemplary Geographic Sources of Index Founder Populations

The following subsections describe exemplary, nonlimiting regions where suitable candidate populations can be found. In some embodiments, suitable candidate populations are descendants (preferably, a direct descendant of people from the geographic regions described below) but do not reside within that geographic region. In some embodiments, geographic location is not used as a criterion for identifying a test population.

5.3.1.1 Kuwait

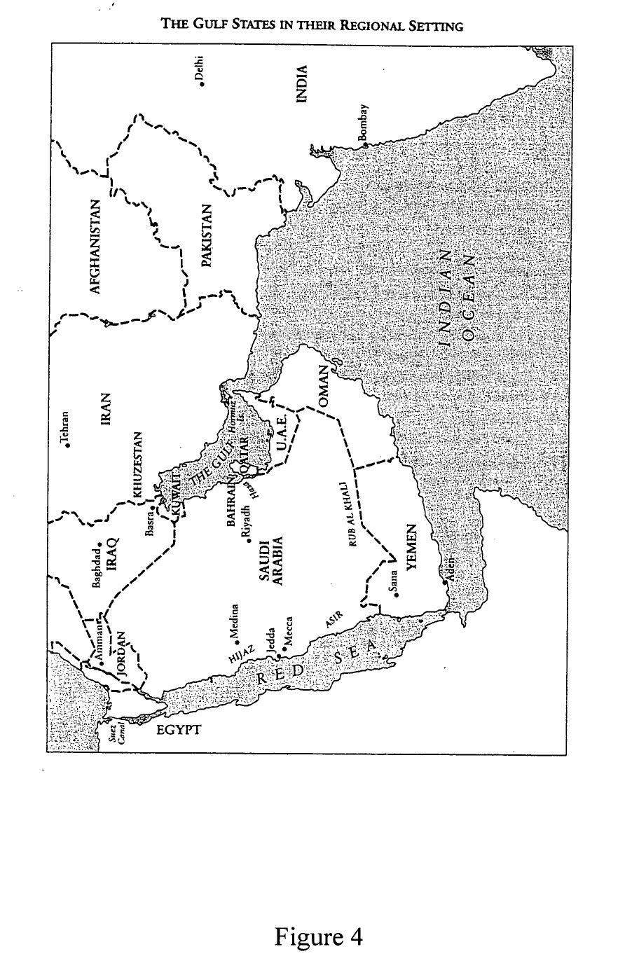

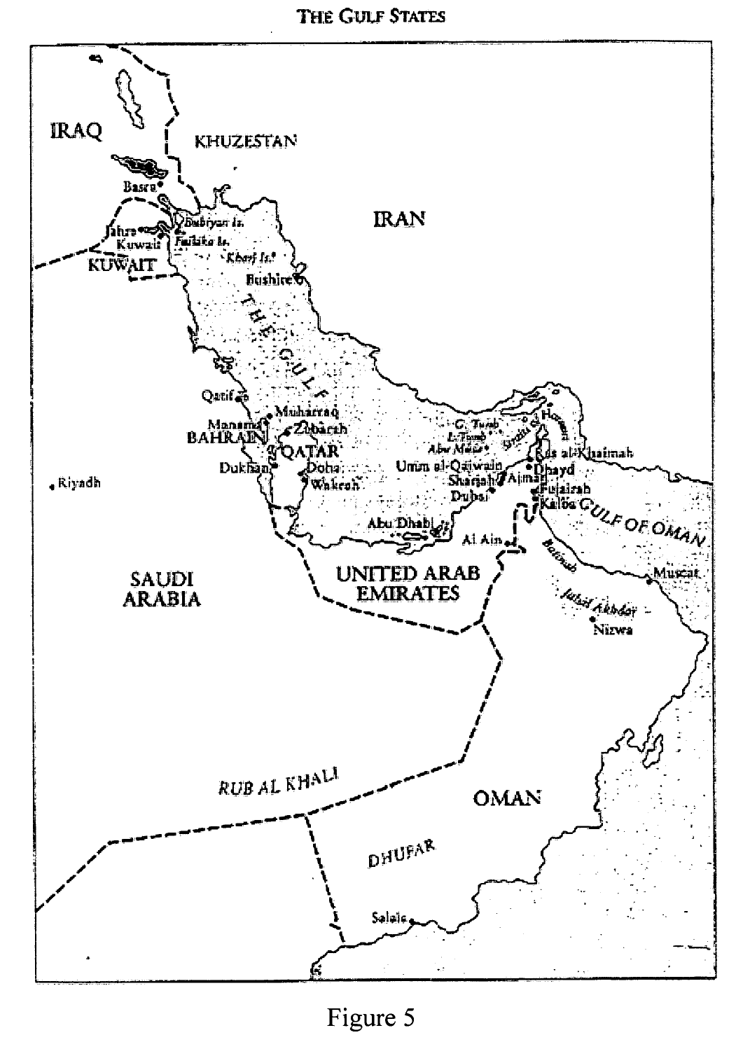

As illustrated in FIGS. 4 and 5, Kuwait is a shaikhdom situated on the western shore of the Arabian gulf. Kuwait was founded in the early eighteenth century by various clans of the Anaiza, who gradually migrated sometime in the late seventeenth century from Nejd to the shores of the Persian Gulf. In the course of these migrations, different tribal groups came together to form a new tribe, that became collectively known as Bani Utub after the migration.

Kuwait is isolated on three sides by vast expanses of desert and on the fourth by the Arabian gulf. Kuwait has been ruled by the same family since 1756. In 1949, Kuwait's population was estimated to be approximately 100,000. Kuwait's population increased by 557 percent between 1957 and 1975, an annual average increase of 24 percent over the twenty-three year period. Foreign immigration constituted the largest component of increase, and by 1965 Kuwaiti nationals constituted a minority in the nation.

The distinction between Kuwaiti nationals and non-Kuwaiti nationals has significance in Kuwait. According to Article 1 of the citizenship law of 1959, Kuwaiti nationality is recognized for those and their descendants who resided in Kuwait before 1920 and maintained residence there in 1959. By 1965, non-Kuwaitis constituted 52.9 percent of the population of Kuwait. As of 2004, the population of Kuwait was 2,257,549, of which 1,291,354 (57%) were non-nationals.

In some embodiments of the present invention, for the purposes of identifying an index founder population, nationals of Kuwait are considered a test population. In some embodiments, one or more additional criteria are imposed. For instance, in some embodiments, only those nationals of Kuwait that are Sunni Muslims are considered a suitable test population for the identification of an index founder population. In still other embodiments, only those nationals of Kuwait that are direct descendants of the Bani Utub are considered a suitable test population for the identification of an index founder population.

5.3.1.2 United Arab Emirates (Abu Dhabi, Dubai)

The United Arab Emirates, also called the UAE, is a Middle Eastern country situated in the south-east of the Arabian Peninsula in Southwest Asia on the Persian Gulf, comprising seven emirates: Abu Dhabi, Ajman, Dubai, Fujairah, Ras al-Khaimah, Sharjah and Umm Al Quwain. Before 1971, they were known as the Trucial States or Trucial Oman. As illustrated in FIGS. 4 and 5, the United Arab Emirates borders Oman and Saudi Arabia.

As of 2005, UAE's population stands at 4.041 million and consists of over 3.23 million non-nationals. Around 50% of the population is South Asian, with the remainder being Emirati, Arab, European and East Asian. Some of the natives are originally of Persian and Indian subcontinent descent. Religious beliefs are mostly Muslim (Islam is the state religion). However, there are sizable minorities of Christians, Hindus and other faiths.

In some embodiments of the present invention, for the purposes of identifying an index founder population, citizens of UAE are considered a test population. In some embodiments, one or more additional criteria are imposed. For instance, in some embodiments, only those citizens of UAE that are Sunni Muslims are considered a suitable test population for the identification of an index founder population.

5.3.1.3 QATAR

According to “The Emergence of Qatar” by Habibur Rahman (Kegan paul, London & New York, 2005, 282 pages), in 1905 Lorimer “estimated the total population of Qatar as 27,000 souls consisting of different tribes, namely, al-Maadhid, al Bu Ainain, al Nin Ali, al Bu Kuwara, al-Mohannedi, al-Kubaisat, al-Dawasir, al-Mani, al-Sulaithi, the Persians, etc.” Further, the al-Bu Kuwara were of Beni Tamimi descent, as were the al-Tahni and al-Maadhid.

Qatar has become one of the newer emirates in the Arabian Peninsula. After domination by Persians for thousands of years and more recently by Bahrain, by the Ottoman Turks, and by the British, Qatar became an independent state on Sep. 3, 1971. Unlike most nearby emirates, Qatar declined to become part of either the United Arab Emirates or of Saudi Arabia. Qatar, officially State of Qatar, independent emirate, is a largely barren peninsula in the Persian Gulf, bordering Saudi Arabia and the United Arab Emirates. See, FIGS. 4 and 5.

As of July 2005, the population of Qatar was 863,051. A minority, twenty percent, of the population of Qatar are Qatari citizens (Arabs of the Wahhabi sect of Islam). The rest of the population is largely other Arabs, Pakistanis, Indians, and Iranians. Qatar explicitly uses Wahhabi law as the basis of its government, and the vast majority of its citizens follow this specific Islamic doctrine. Muhammad ibn Abd al-Wahhab founded Wahhabism, a puritanical version of Islam which takes a literal interpretation of the Koran, also known as the Qu'aran and the Sunnah.

In some embodiments of the present invention, for the purposes of identifying an index founder population, citizens of Qatar are considered a test population. In some embodiments, one or more additional criteria are imposed. For instance, in some embodiments, only those citizens of Qatar that practice Wahhabism are considered a suitable test population for the identification of an index founder population.

5.3.1.4 Yemen

North Yemen became independent of the Ottoman Empire in 1918. The British, who had set up a protectorate area around the southern port of Aden in the 19th century, withdrew in 1967 from what became South Yemen. Three years later, the southern government adopted a Marxist orientation. The exodus of hundreds of thousands of Yemenis from the south to the north contributed to two decades of hostility between the states. The two countries were formally unified as the Republic of Yemen in 1990. A southern secessionist movement in 1994 was quickly subdued. Religions represented in Yemen include Muslim (e.g., Shaf'i (Sunni) and Zaydi (Shi'a)) and, to a lesser extent, Judaism, Christianity, and Hinduism. As of 2002, Yemen had an estimated population of 19,912,000.

In some embodiments of the present invention, for the purposes of identifying an index founder population, citizens of Yemen are considered a test population. In some embodiments, one or more additional criteria are imposed. For instance, in some embodiments, only those citizens of Yemen that practice Shaf'i are considered a suitable test population for the identification of an index founder population. In some embodiments, only those citizens of Yemen that practice Zaydi are considered a suitable test population for the identification of an index founder population.

5.3.1.5 Saudi Arabia

The Kingdom of Saudi Arabia is the largest country on the Arabian Peninsula. As illustrated in FIGS. 4 and 5, it borders Jordan on the north, Iraq on the north and north-east, Kuwait, Qatar, Bahrain, and the United Arab Emirates on the east, Oman on the south and south-east, and Yemen on the south, with the Persian Gulf to its north-east and the Red Sea to its west.

The Saudi state began in central Arabia in about 1750. Saudi Arabia's 2003 population was estimated to be about 24.3 million, including about 6.4 million resident foreigners. Until the 1960s, most of the population was nomadic or semi-nomadic; due to rapid economic and urban growth, more than 95% of the population now is settled. Most Saudis are ethnically Arab. Some are of mixed ethnic origin and are descended from Turks, Iranians, Malays, and others, most of whom immigrated as pilgrims and reside in the Hijaz region along the Red Sea coast. One hundred percent of the citizens of Saudi Arabia are Muslim.

In some embodiments of the present invention, for the purposes of identifying an index founder population, citizens of Saudi Arabia are considered a test population. In some embodiments, one or more additional criteria are imposed. For instance, in some embodiments, only those citizens of Saudi Arabia that can trace their lineage to a family that has been in Saudi Arabia more than twenty, thirty, forty, fifty, sixty, seventy, or eighty years is considered a test population for purposes of identifying an index founder population.

5.3.1.6 Oman

As illustrated in FIGS. 4 and 5, only the northernmost tip of Oman lies on the Gulf. The rest of the country borders the Gulf of Oman and consist of the inland Hajar mountain range; the coastal areas which stretch over 1,600 kilometers from the Gulf to the Gulf of Oman, the Arabian Sea and beyond to the Indian Ocean; and Rub al-Khali desert. This desert acts as a barrier to the rest of the Arabian peninsula.

As of July 2004, the population of Oman is 2,903,165, including 577,293 non-nationals. Most Omanis, particularly those in the interior, are Ibadis, a brand of the oldest sect in Islam. Because the Ibadis are outside mainstream Islamic society—elsewhere they are only to be found in parts of North and East Africa—this has tended to isolate the country further.

In some embodiments of the present invention, for the purposes of identifying an index founder population, citizens of Oman are considered a test population. In some embodiments, one or more additional criteria are imposed. For instance, in some embodiments, only those citizens of Oman that can trace their lineage to a family that has been in Oman more than twenty, thirty, forty, fifty, sixty, seventy, or eighty years is considered a test population for purposes of identifying an index founder population. In some embodiments, only those citizens of Oman that are also Ibadis is considered a test population for purposes of identifying an index founder population.

5.3.1.7 India

The Indus Valley civilization, one of the oldest in the world, goes back at least 5,000 years. Aryan tribes from the northwest invaded about 1500 B.C.; their merger with the earlier inhabitants created classical Indian culture. Formerly an English colony, India gained independence in 1947.

In 2001, the population of India was estimated to be 1,029,991,145. Ethnic groups include Indo-Aryans 72%, Dravidians 25%, Mongoloid and others 3%. Religions include Hindu 81.3%, Muslim 12%, Christian 2.3%, Sikh 1.9%, and other groups including Buddhist, Jain, and Parsi 2.5% and Judaism. Languages include Bengali (official), Telugu (official), Marathi (official), Tamil (official), Urdu (official), Gujarati (official), Malayalam (official), Kannada (official), Oriya (official), Punjabi (official), Assamese (official), Kashmiri (official), Sindhi (official), Sanskrit (official), and Hindustani (a popular variant of Hindi/Urdu spoken widely throughout northern India).

In some embodiments of the present invention, for the purposes of identifying an index founder population, citizens of India that are of Indo-Aryans heritage are considered a test population. In some embodiments, for the purposes of identifying an index founder population, citizens of India that are Dravidians are considered a test population. In some embodiments, for the purposes of identifying an index founder population, citizens of India that are Mongoloid are considered a test population. In some embodiments, one or more additional criteria are imposed in the selection of a test population. For instance, in some embodiments, only those citizens of India that speak a particular one of the official languages of India are considered a test population. In one example, only those citizens of India that speak Bengali are considered for a given test population from which an index founder population is derived.

Another criterion that can be used to select a test population is religion. In some embodiments, only those citizens of India that are Hindu are considered a test population. In other embodiments, only citizens of India that are Jain are considered a test population. In other embodiments, only citizens of India that are Parsi are considered a test population. In yet other embodiment only citizens of India that are Sikh are considered a test population.

In Hinduism there are four castes, which in order from the highest to lowest caste are Brahman, Kshataria, Vaisia and Sudra. Members of the Kshataria caste are the rulers and aristocrats of the society. Members of the Vaisia caste are the landlords and businessmen of the society. Members of the Sudra caste are the peasants and working class of the society. Below the four castes are the untouchables.

Another criterion that can be used to select a test population is caste. Although the caste system is illegal in India, many people marry within their caste.

Each caste and the untouchables are divided into many communities known as Jat or Jati. For example, the Brahmans have Jats call Gaur, Kokanashtha, Sarasvat, Iyer, and others. In some embodiments, only citizens of India that belong to a particular caste are considered a test population. In other embodiments, only citizens that belong to a particular Jat or Jati within a particular caste are considered a test population.

Another criterion that can be used to select a test population is geographic location within India. In some embodiments, only citizens of India that reside in or trace their ancestry to a particular state in India are considered a test population. In other embodiments, only citizens of India that reside in or trace their ancestry to a particular region within a particular state in India are considered a test population.

5.3.2 Factors for Defining Index Founder Populations

The populations identified in Section 5.3.1 provide a nonlimiting source of test populations that can be further screened in order to identify index founder populations suitable for use in the present invention. In some embodiments, however, the test population is not limited to a specific geographical area. Thus, in some embodiments, step 202 in Section 5.2 is directed to finding a test population that is not associated with a specific geographical area (e.g., a nomadic population). In some embodiments, identification of test populations, such as those described in Section 5.3.1, is done by asking willing participants to fill out a questionnaire. In some embodiments, additional factors are used to identify a suitable population for use in the disclosed systems and methods. Chief among these factors is the degree of consanguinity. In some embodiments, a test population identified in Section 5.3.1 is validated as an index founder population based on the consanguinity of the population.

Consanguinity can be the result of social considerations such as caste systems, political considerations, etc. Presence of a high degree of consanguinity in a test population (e.g., a population identified in Section 5.3.1) is preferred because it serves to further isolate a gene pool and therefore facilitates the association of clinical traits in such a population with candidate chromosomal regions. Consanguinity is defined as marriage between second cousins or more closely related individuals (Teebi and El-Shanti, 2006, Lancet: 367: 970-917). Thus, the percent consanguinity (consanguinity rate) of a population or a generation of the population is the percentage of marriages in the population or the generation of the population that are consanguineous.

Marriage between related kin in the past and/or present can be dictated by a limited number of available individuals as in the case of an index founder population.

Alternatively, consanguinity can also be prescribed by strict cultural practice or religious doctrine. Both types of situations have created IFPs throughout the world that may be useful to study complex disease. In particular, close-kin marriage is often practiced within populations of the Middle East. As set forth in Table, 1, consanguinity rates among Middle Eastern countries are remarkably high and range widely from 20-70% (Teebi and El-Shanti, 2006, Lancet: 367: 970-917). See Table 1 for consanguinity break down in each country.

In contrast to the countries in Table 1, many countries have consanguineous marriage rates of less than one percent including the United States, Canada, Mexico, Russia, Australia, and Argentina. Further still, many countries have consanguineous marriage rates of less than four percent including Brazil and China. Thus, consanguineous marriage rates on a per country basis in the world exhibit a bimodal distribution with many countries having a rate of less than four percent and many countries having a rate of ten percent or greater.

TABLE 1
Consanguinity rates in the Middle East

| Consanguinity rate | Country | Year |
|---|---|---|
| 54.50% | Qatar | 2006 |
| 68% | Egypt | 2001 |
| 33% | Syria | 1974 |
| 51.2-58.1% | Jordan | 1992/2003 |
| 54.40% | Kuwait | 1985 |
| 57.70% | Saudi Arabia | 1995 |
| 50.50% | UAE | 1996/1997 |
| 40-47% | Yemen | 2003/2004 |
| 35.90% | Oman | 2000 |
| 64% | Israel | 2004 |
| 40% | Algeria | 1992 |
| 23% | Algeria | 1984 |
| 37.40% | Egypt | 1993 |
| 41% | Egypt | 1989 |
| 23.30% | Egypt | 1989 |
| 28.96% | Egypt | 1983 |
| 24.50% | Iran | 1979 |
| 57.87% | Iraq | 1986 |
| 50.23% | Jordan | 1992 |
| 36.20% | Jordan | 1989 |
| 53.00% | Kuwait | 1991 |
| 37.80% | Kuwait | 1989 |
| 54.30% | Kuwait | 1985 |
| 25% | Lebanon | 1989 |
| 26% | Lebanon | 1984 |
| 29% | Morocco | 1992 |
| 33% | Morocco | 1987 |
| 57.70% | Saudi Arabia | 1995 |
| 54.30% | Saudi Arabia | 1990 |
| 33% | Syria | 1974 |
| 49% | Tunisia | 1988 |
| 20% | Turkey | 1992 |
| 21.21% | Turkey | 1988 |
| 50.50% | UAE | 1997 |
| 29% | Iraq | 1989 |
| 30% | Kuwait | 1985 |
| 26% | Saudi Arabia | 1990 |
| 24% | Oman | 2000 |
| 32% | Jordan | 1992 |
| 66% | Jordan | 1993 |
| 25.60% | Jordan | 2005 |

Given the relationship of the offspring's parents, the percentage of consanguinity and amount of inherited homozygous loci in the offspring can be predicted (Lander and Botstein, 1987, Science 236: 1567-1570). History of consanguinity over a number of generations will influence the percentage of the genome that is homozygous by descent (Table 2).

Table 2. Levels of Consanguinity with Expected Fraction of Homozygous Loci

TABLE 2
Levels of consanguinity with expected
fraction of homozygous loci

| Level | Relationship (offspring of:) | Expected homozygous loci |
|---|---|---|
| 1 | double first cousin, uncle-niece | ⅛ |
| 2 | first cousin | 1/16 |
| 3 | second cousin | 1/64 |
| 4 | less than second cousin | less than 1/64 |

It has been demonstrated that the theoretical prediction of homozygous loci in offspring from first cousin marriage (6%) is accurate in a population with recent consanguinity (Woods et al., 2006, Am J. Hum Genet. 78: 889-896). However, this study also revealed that multiple generations of consanguinity created a greater amount of homozygosity in offspring from first cousin unions than predicted.

In some embodiments, a population is deemed to be consanguineous if the consanguinity rate of the population is ten percent or greater. In other words, a population is deemed to be consanguineous if more then ten percent of the population are offspring of a level 2 or closer (e.g., level 1) relationship. In some embodiments, a population is deemed to be consanguineous if the consanguinity rate of the population is twenty percent or greater, thirty percent or greater, forty percent or greater, fifty percent or greater, or sixty percent or greater. In some embodiments, a population is considered consanguineous if the average coefficient of inbreeding F_{avg} in the population is 0.10 or greater, 0.12 or greater, 0.14 or greater, 0.16 or greater, 0.18 or greater, or 0.20 greater. Here, the coefficient of inbreeding F is defined as the chance that a given locus in a subject in the population will be found homozygous by descent or, equivalently, the fraction of the subject's genome expected to be homozygous by descent. F_{avg} is the value of F averaged across all the members of the population. See, for example, Wright, 1922, Am. Nat. 56, 330, which is hereby incorporated by reference herein for the purpose of describing the coefficient of inbreeding. In some embodiments, the coefficient of inbreeding F for a given subject in the population is limited to considering the relationship between the given subject's parents. For example, if the subject is a product of sibling, first-cousin, second-cousin, or unrelated marriage, F=¼, 1/16, 1/64, and 0 respectively. The value F for each subject in the population is then averaged to compute the average coefficient of inbreeding F_{avg} in the population. In some embodiments, the coefficient of inbreeding F for a given subject in the population is limited to considering the relationship between the given subject's parents as well as grandparents.

In some embodiments, for the purpose of identifying an IFP, populations enrolled in a study can be assigned a degree of consanguinity (DC) based upon knowledge of parental relationships in that group in accordance with Table 3. In Table 3, the degree of consanguinity ranges from 0% to over 50% and is equated with a score for the purpose of ranking an IFP.

A second criterion for ranking the IFP using consanguinity data relies on the modality (MC) of the consanguinity in the sample. For example, in one embodiment, first cousin union of parents results in an MC score of 512 in the sample. The modality score of each subject in the population is summed and then averaged by the number of persons in the population in order to calculate an average modality score. This average modality score can then be added to the DC score (degree of consanguinity) for the population in order to arrive at a final score for the population. In some embodiments that use the summation of the modality score and the degree of consanguinity score using the assignments given for such scores in Table 3, a population identified using the techniques disclosed in Section 5.3.1 are considered consanguineous when the score is 200 or greater, 225 or greater, 250 or greater, 275 or greater, 300 or greater, 325 or greater, 350 or greater, 375 or greater, or 400 or greater.

As discussed in more detail below, factors over and above consanguinity, such as average family size and number of generations available, can also be used to assist in validation a population identified in Section 5.3.1 as an index founder population. In some embodiments, arithmetic addition of scores of variables such family size and number of generations available are factored in with the consanguinity scores (DC and/or MC) for final ranking. It will be appreciated that the actual scores assigned to particular population factors in Table 3 is just one of many possible scoring systems. For instance, scoring systems in which a lower score indicates that a population is an IFP are within the scope of the present invention.

TABLE 3
Expanded IFP rating scheme

| Population Factor | Symbol | Score |
|---|---|---|
| Degree of Consanguinity: | DC | |
| 0% to 2% pop. DC | DC.00.02 | 1 |
| 2% to 4% pop. DC | DC.02.04 | 2 |
| 4% to 6% pop. DC | DC.04.06 | 3 |
| 6% to 8% pop. DC | DC.06.08 | 4 |
| 8% to 10% pop. DC | DC.08.10 | 5 |
| 10% to 20% pop. DC | DC.10.20 | 16 |
| 20% to 30% pop. DC | DC.20.30 | 64 |
| 30% to 40% pop. DC | DC.30.40 | 256 |
| 40% to 50% pop. DC | DC.40.50 | 512 |
| Over 50% pop. DC | DC50.100 | 1024 |
| Modality of Consanguinity in | MC | |
| Sample selection: | | |
| Both Parents and grandparent 1st | MC.P1.GP1 | 1024 |
| Cousins | | |
| Both Parents 1^{st} Cousins | MC.P.1C.1C | 512 |
| Both Grandparents 1^{st} Cousins | MC.GP.1C.1C | 256 |
| Both Parents and GP 2nd Cousins | MC.P2.GP2 | 5 |
| Both Parents 2nd Cousins | MC.P.2C.2C | 4 |
| Both Grandparents 2^{nd} Cousins | MC.GP.2C.2C | 3 |
| Average Family Size: | AFS | |
| One Child | AFS.1 | 1 |
| Two Children | AFS.2 | 2 |
| Three Children | AFS.3 | 3 |
| Four Children | AFS.4 | 4 |
| Five or more Children | AFS.5 | 5 |

In some embodiments, one or more factors over and above consanguinity are used to select an index founder population out of a test population. Such factors include, but are not limited to, average family size, availability of medical records, occupation of same region, degree of genetic isolation, availability of historical records, availability of historical population and demographic data, family structure (polygamous versus monogamous), generations in a single household, life expectancy, nomadic versus agriculture-based, availability of medical records, accessibility/willingness of the population, and patriarchy/matriarchy considerations

Average family size. Larger families are preferred because such families provide more genetic information for some forms of quantitative phenotype analysis than smaller families.

Occupation of same region. The presumption behind this factor is that populations that have stayed in the same geographic region for multiple generations will have a higher degree of genetic isolation than those populations that have not.

Availability of medical records. In one embodiment, there are comprehensive medical records available for all or a portion of the members of an index founder population. Such medical records provide a rich source of clinical traits that can be associated with candidate chromosomal regions. In other embodiments, there are no comprehensive medical records available for an index founder population.

Accessibility/willingness of the population. Those populations that are cooperative and are committed to providing answers to the questionnaires as well as providing biological sample are preferred over populations that are not willing.

5.3.3 Indexing Systems

Section 5.3.1 provided examples of geographic sources of index founder populations that can form the basis of a test population. In some embodiments, an index founder population that is used to link one or more candidate chromosomal regions with a candidate clinical trait is selected from the test populations of Section 5.3.1 using one or more additional selection criteria, such as any combination of the factors described in Section 5.3.2. For example, in some embodiments, a suitable Kuwaiti population identified in Section 5.3.1 is filtered using a factor described in Section 5.3.2 in order to derive a suitable in index founder population. Sections 5.3.3.1 and 5.3.3.2, below, describe methods by which the factors described in Section 5.3.2 can be used to screen a test population in order to obtain an index founder population.

5.3.3.1 Arithmetic Sum of Rated Factors

In Section 5.3.2, numerous factors that can be used to screen a population were described. In some embodiments, members of a test population are screened against one or more factors. For each of these factors, subjects in the test population are assigned a score. In one embodiment, lower scores represent undesirable attributes whereas higher scores represent more preferred attributes. In some embodiments, a score of “1” is assigned for a given factor for a subject when the attribute exhibited by the subject for the given factor is not suitable for the purposes of the present invention and a score of “5” when it is highly suitable. An intermediate value of “2”, “3” or “4” is assigned when the attribute falls somewhere between these two extremes. For example, consider a subject in a test population. A factor that is being used to refine the test population into an index founder population is degree of consanguinity. If the subject has a coefficient of inbreeding F of, for example, 0.05 or less, a low degree of consanguinity is assigned to the subject (e.g. “1”). However, if the subject has coefficient of inbreeding of 0.25, the subject is assigned a consanguinity score of “5”. Coefficients of inbreeding between 0.05 and 0.25 are assigned consanguinity scores between 1 and 5. To compute the consanguinity score for the test population in this embodiment, scores assigned to each subject in the test population are averaged.

In some embodiments, values for factors are assigned to an entire test population as opposed to individual members of the test population. For example, in some embodiments, the degree of consanguinity of the test population as a whole is considered. If the degree of consanguinity is high, then the test population itself is assigned a high value for this factor. In other embodiments, values for each factor are assigned to each respective member by considering the respective member in the context of the respective member's family. Table 4 illustrates a scoring scheme for some factors that can be used to screen a population and examples of possible scores that can be assigned to subjects for each of these factors.

TABLE 4

| FACTOR | SYMBOL | LOW SCORE - 1 | HIGH SCORE - 5 |
|---|---|---|---|
| Availability medical records | AMR | not available | available |
| Degree of consanguinity | DC | low degree of | high degree of |
| | | consanguinity in family | consanguinity in |
| | | | family |
| Modality of | MC | Two grandparents | both Parents and |
| consanguinity | | second cousins | Grandparents first |
| | | | cousins |
| Average family size | AFS | one child | five more children |
| Number of generations | NGGR | First generation | five or more |
| that the population has | | immigrant | generations |
| occupied the same | | | |
| geographic region | | | |
| Accessibility/ | AW | not willing | very willing |
| willingness | | | |
| Degree of genetic | DGI | no evidence of | extremely |
| Isolation | | genetic isolation | genetically isolated |
| Availability of | AHP | no records | extensive records |
| historical population | | | |
| and demographic data | | | |
| Family structure | FS | Monogamous | polygamous |
| (polygamous versus | | | |
| monogamous) | | | |
| Number of generations | NG | one generation | three or more |
| in single household | | | generations |
| Life expectancy | LE | less than 65 years | more than 70 years |
| | | at birth | at birth |
| Nomadic versus | NO | nomadic | agricultural-based |
| agricultural-based. | | | |

In some embodiments, a score is assigned to each respective member of a test population and only those members of the test population that receive a score higher than a threshold score are considered part of the index founder population. For example, consider the case in which one of the test populations described in Section 5.3.1 has been identified. In this example, only those members of the test population that receive a score greater than 10 are selected for the index founder population, using the factors AMR, DC, and AFS. Thus if a subject has a value of 3 for AMR, 2 for DC and 5 for AFS, for a total 10, the subject will not be selected for the index founder population. On the other hand, a subject having a value of 4 for AMR, 2 for DC, and 5 for AFS, the subject will be selected for the index founder population.

In another example, consider the case in which one of the test populations described in Section 5.3.1 has been identified. In this example, only those members of the test population that receive a score greater than 15 are selected for the index founder population, using the factors FS, NG, LE, and NO. Thus, if a subject has a value of 3 for FS, 2 for NG, 5 for LE, and 2 for NO, for a total 12, the subject will not be selected for the index founder population. On the other hand, if a subject has a value of 5 for FS, 5 for NG, 5 for LE, and 2 for NO, for a total 16, the subject will be selected for the index founder population.

It can be appreciated that any desired combination of factors and any threshold value can be used in the selection of an index founder population from a test population. For example, any combination of 2, 3, 4, 5, 6, 7, 9, or 10 factors from the table above can be used in such a decision making process. Each subject can be assigned a value for a factor that depends on how closely the subject adheres to the goal of the factor in identifying genetically isolated subjects suitable for an index founder population. A scale having a range of 1 to 5 for each factor has been described and presented in the table above. However, the invention is not so limited. In some embodiments, each subject is assigned a “+” or “−” for each factor, and the subject is selected for the index founder population if more of the factors are assigned a “+” than are assigned a “−”. Such embodiments require that an odd number of factors be considered or, alternatively, rules for what happens when there is an equal number of plusses and minuses.

In embodiments in which factor values are assigned to the population as a whole, rather than individual subjects, the use of the factor assignments serves to validate the test population as an index founder population. Thus, for example, if the summed score of the factor values assigned to the population does not exceed a threshold value, the test population is rejected and another test population is sought for validation. On the other hand, if the summed score of the factor values assigned to the population does exceed the threshold value, the test population is selected (validated) as an index founder population. To illustrate, consider the case in which the threshold score is 15 using the factors FS, NG, LE, and NO. If a test population has a value of 3 for FS, 2 for NG, 5 for LE, and 2 for NO, for a total 12, the test population will not be selected as an index founder population. On the other hand, if a test population has a value of 5 for FS, 5 for NG, 5 for LE, and 2 for NO, for a total 16, the test population will be selected as an index founder population.

5.3.3.2 Weighting Schemes

Not every factor used to facilitate the selection of an index founder population from a test population needs to be given equal weight. For example, as discussed above, consanguinity is considered to be the most important factor. Accordingly, in some embodiments, weights are assigned to each factor. For example, consider the case in which there are three factors, F₁, F₂, and F₃ used to screen a test population, where each F is any factor, such as a factor from the Table in Section 5.3.3.1. Each factor is independently assigned a weight W that is commensurate with the discriminating value of the weight. Then, the weighted sum of the factors is computed in order to determine whether a subject exceeds a predetermined threshold value.

To illustrate a weighting scheme, consider the case in which one of the test populations described in Section 5.3.1 has been identified. In this example, only those members of the test population that receive a score greater than 15 are selected for the index founder population, using the factors FS, NG, LE, and NO. FS has a weight of 1, NG has a weight of 2, LE has a weight of 0.5, and NO has a weight of 0.1. Thus,
Score=1*[FS]+2*[NG]+0.5*[LE]+0.1*[NO].
If a subject has a value of 3 for FS, 2 for NG, 5 for LE, and 2 for NO, then the score for the subject will be:
Score=1*[3]+2*[2]+0.5*[5]+0.1*[2]=9.7
Thus, the subject will not be selected for the index founder population given the weighting scheme.

5.3.3.3 Confirmatory Scoring Schemes Based on Genotypes

Methods for identifying an index founder population in a test population using one or more factors, either as a weighted or an unweighted arithmetic sum, have been described. In some embodiments, genotypic screening is used to identify an index founder population in a test population. Such genotypic screening can be used in combination with the factor approach. In fact, a quantitative measure associated with genotype can serve as one of the factors used in the approaches described in Sections 5.3.3.1 and 5.3.3.2. In some embodiments, the genotypic approach is used instead of the factor based screening described in Sections 5.3.3.1 and 5.3.3.2.

Biological samples are obtained from subjects in the test population in accordance with Section 5.4.1 and genotyped in accordance with Section 5.4.2. In this way, genotypic information for a set of markers (e.g. SNPs) is obtained. Such genotypic information can be used to determine the genetic relatedness of the test population. For instance, the genotypic information can be used to determine whether the test population is best explained as one discrete population, an admixture of two discrete populations, three populations, etc.

One embodiment of the present invention provides a method of associating a clinical parameter with one or more candidate chromosomal regions in the human genome. In the method an index founder population is identified in a test population based upon the genotype X of each member of the test population, where the posterior probability Pr(K|X) for the index founder population is greatest for K=1, and where K represents the number of subpopulations in the index founder population. In some embodiments, the test population has multiple subpopulations K and a single subpopulation in the K subpopulations is selected as the index founder population. Next, the clinical parameter is measured from each respective member of the index founder population. Then, a quantitative trait locus analysis between (i) the genotype X of each member of the index founder population and (ii) the clinical parameter is performed using, for example, the techniques disclosed in Sections 5.6 or 5.7 below to thereby identify one or more candidate chromosomal regions in the human genome that associate with the clinical parameter.

There are many ways in which actual subpopulations of a test population can be identified and individuals assigned, on a probabilistic basis, to these subpopulations. In some embodiments, a Bayesian clustering approach is used. See, for example, Pritchard et al., 2000, Genetics 155: 945-959, which is hereby incorporated by reference herein in its entirety. Assume a model in which there are K subpopulations (where K may be unknown), each of which is characterized by a set of allele frequencies at each locus. The approach attempts to assign individuals in the test populations to the subpopulations on the basis of their genotypes, while simultaneously estimating population allele frequencies. The method can be applied to various types of markers [e.g., microsatellites, restriction fragment length polymorphisms (RFLPs), or single nucleotide polymorphisms (SNPs)], but it assumes that the marker loci are unlinked and at linkage equilibrium with one another within populations. It also assumes Hardy-Weinberg equilibrium within populations. The approach allows for the presence of admixed individuals in the test population, whose genetic makeup is drawn from more than one of the K subpopulations.

In the method, genetic data from samples of individuals in the test population are taken and clustered. Individuals who are genetically similar form distinct clusters. In some embodiments, a check is made to determine whether clusters relate to geographical or phenotypic data on the individuals. There are broadly two types of clustering methods that can be used (i) distance-based methods and (ii) model-based methods.

Distance-based methods. Distance-based methods proceed by calculating a pairwise distance matrix, whose entries give the distance (suitably defined) between every pair of in-situ individuals. This matrix may then be represented using convenient graphical representation, such as a multidimensional scaling plot, and clusters may be identified by eye.

Model-based methods. Model-based methods proceed by assuming that observations from each cluster are random draws from some parametric model. Inference for the parameters corresponding to each cluster is then done jointly with inference for the cluster membership of each individual, using standard statistical methods (for example, maximum-likelihood or Bayesian methods).

Distance-based methods are usually easy to apply and are often visually appealing. In the genetics literature, it has been common to adapt distance-based phylogenetic algorithmns, such as neighbor-joining, to clustering multilocus genotype data. However, these methods suffer from many disadvantages: the clusters identified may be heavily dependent on both the distance measure and graphical representation chosen; it is difficult to assess whether the clusters obtained in this way are meaningful; and it is difficult to incorporate additional information such as the geographic sampling locations individuals. Distance-based methods are thus more suited to exploratory data analysis than to fine statistical inference, and in preferred embodiments of the present invention, a model-based approach is taken.

The first challenge when applying model-based methods is to specify a suitable model for observations from each cluster. Assume that each cluster K (subpopulation) is modeled by a characteristic set of allele frequencies. Let X denote the genotypes of the sampled individuals of the test population, Z denote the (unknown) populations of origin of the individuals, and P denote the (unknown) allele frequencies in all populations. (Note that X, Z, and P actually represent multidimensional vectors.) The main modeling assumptions are Hardy-Weinberg equilibrium within populations and complete linkage equilibrium between loci within populations. Under these assumptions each allele at each locus in each genotype is an independent draw from the appropriate frequency distribution, and this completely specifies the probability distribution Pr(X|Z, P). The idea here is that the model accounts for the presence of Hardy-Weinberg linkage disequilibrium by introducing population structure and attempts to find population groupings that, as far as possible, are not in disequilibrium. While inference may depend heavily on these modeling assumptions, it is easier to assess the validity of explicit modeling assumptions than to compare the relative merits of more abstract quantities such as distance measures and graphical representations. In situations where these assumptions are deemed unreasonable then alternative models can be built.

Having specified the model, a decision is needed on how to perform inference for the quantities of interest (Z and P). Here, a Bayesian approach has been taken, although other approaches are suitable by specifying models (priors) Pr(Z) and Pr(P), for both Z and P. The Bayesian approach provides a coherent framework for incorporating the inherent uncertainty of parameter estimates into the inference procedure and for evaluating the strength of evidence for the inferred clustering. It also eases the incorporation of various sorts of prior information that may be available, such as information about the geographic sampling location of individuals.

Having observed the genotypes, X, knowledge about Z and P is given by the posterior distribution:
Pr(Z,P|X)∝Pr(Z)Pr(P)Pr(X|Z,P)
While it is not usually possible to compute this distribution exactly, it is possible to obtain an approximate sample (Z⁽¹⁾, P⁽¹⁾), (Z⁽²⁾, P⁽²⁾), . . . ,(Z^{(M)}, P^{(M)}) from Pr(Z,P|X) using Markov chain Monte Carlo (MCMC) methods described in Pritchard et al., 2000, Genetics 155, 945-959; as well as Gilks et al., 1996, Markov Chain Monte Carlo in Practice, Chapman & Hall, London, each of which is hereby incorporated by reference herein in its entirety.) Inference for Z and P may then be based on summary statistics obtained from this sample.

The problem of inferring the number of subpopulations, K, in a given test population will now be addressed. Using a Bayesian paradigm a prior distribution is placed on K inference for K is based on the posterior distribution:
Pr(K|X),∝Pr(X|K)Pr(K).
However, this posterior distribution can be peculiarly dependent on the modeling assumptions made, even where the posterior distributions of other quantities (Q, Z, and P, say) are relatively robust to these assumptions. Moreover, there are typically severe computational challenges in estimating Pr(K|X) . Therefore, in some embodiments Pr(K|X) is estimated using the ad hoc rule:

$$Pr ( X \vert K ) \approx exp ( - \hat{μ} / 2 - {\hat{σ}}^{2} / 8 )$$

$$where$$

$$\hat{μ} = \frac{1}{M} {\sum }_{m = 1}^{M} - 2 log Pr ( X \vert {Z}^{( m )} , {P}^{( m )} , {Q}^{( m )} )$$

$$and$$

$${\hat{σ}}^{2} = \frac{1}{M} {\sum }_{m = 1}^{M} {( - 2 log Pr ( X \vert {Z}^{( m )} , {P}^{( m )} , {Q}^{( m )} ) - \hat{μ} )}^{2} .$$

the estimate for Pr(X|K) is used for each K and these estimates are substituted into Pr(K|X)∝Pr(X|K) Pr(K) to approximate the posterior distribution Pr(K|X) . In some embodiments, an index founder population is identified from a test population when the posterior probability Pr(K|X) for the index founder population for any K less than 6 and greater than 1 is 0.4 or less. In some embodiments, an index founder population is identified from a test population when the posterior probability Pr(K|X) for the index founder population for any K less than 6 and greater than 1 is 0.3 or less.

5.4 Germ Line Assay

A germ line assay is performed on each subject of a population that has been identified using the methods of the present invention. One or more biological samples is obtained from each subject in order to conduct the germ line assay. Representative biological samples are described in Section 5.4.1, below. Genotyping is then performed with the biological samples. In some embodiments, the biological samples are used to sequence a portion of the human genome. Representative genotyping techniques used in some embodiments of the present invention are described in Section 5.4.2, below.

5.4.1 Biological Samples

Samples from a subject used in accordance with the invention for genotyping and sequencing of the genome or portion thereof include biological samples and samples derived from a biological sample which comprise genomic DNA (i.e., a “genotyping biological sample”). In certain embodiments, in addition to the biological sample itself or in addition to material derived from the biological sample such as cells and genomic DNA, the sample used in the methods of this invention comprises added water, salts, glycerin, glucose, an antimicrobial agent, paraffin, a chemical stabilizing agent, heparin, an anticoagulant, or a buffering agent.

In accordance with the invention, a sample derived from a biological sample is one in which the biological sample has been subjected to one or more pretreatment steps prior to genotyping and/or sequencing. In certain embodiments, a biological fluid is pretreated by centrifugation, filtration, precipitation, dialysis, or chromatography, or by a combination of such pretreatment steps. In other embodiments, a tissue sample is pretreated by freezing, chemical fixation, paraffin embedding, dehydration, permeablization, or homogenization followed by centrifugation, filtration, precipitation, dialysis, or chromatography, or by a combination of such pretreatment steps. In certain embodiments, the sample is pretreated by adjusting the concentration of nucleic acid in the sample, by adjusting the pH or ionic strength of the sample, or by removing contaminating proteins, nucleic acids, lipids, or debris from the sample prior to genotyping and/or sequencing.

In a specific embodiment, the sample is a blood sample. A blood sample may be obtained from a subject according to methods well known in the art. In some embodiments, a drop of blood is collected from a simple pin prick made in the skin of a subject. In such embodiments, this drop of blood collected from a pin prick is all that is needed. Blood may be drawn from a subject from any part of the body (e.g., a finger, a hand, a wrist, an arm, a leg, a foot, an ankle, a stomach, and a neck) using techniques known to one of skill in the art, in particular methods of phlebotomy known in the art. In a specific embodiment, venous blood is obtained from a subject and utilized in accordance with the methods of the invention. In another embodiment, arterial blood is obtained and utilized in accordance with the methods of the invention. The composition of venous blood varies according to the metabolic needs of the area of the body it is servicing. In contrast, the composition of arterial blood is consistent throughout the body. For routine blood tests, venous blood is generally used.

Venous blood can be obtained from the basilic vein, cephalic vein, or median vein. Arterial blood can be obtained from the radial artery, brachial artery or femoral artery. A vacuum tube, a syringe or a butterfly may be used to draw the blood. Typically, the puncture site is cleaned, a tourniquet is applied approximately 3-4 inches above the puncture site, a needle is inserted at about a 15-45 degree angle, and if using a vacuum tube, the tube is pushed into the needle holder as soon as the needle penetrates the wall of the vein. When finished collecting the blood, the needle is removed and pressure is maintained on the puncture site. Usually, heparin or another type of anticoagulant is in the tube or vial that the blood is collected in so that the blood does not clot. When collecting arterial blood, anesthetics can be administered prior to collection.

In some embodiments of the present invention, blood is collected and/or stored in a K₃/EDTA tube. In a specific embodiment, blood is collected and/or stored in ACD-A tubes (Becton Dickinson Catalog No. 364606). In another embodiment, blood is collected and/or stored on one, two, three, four or more FAST TECHNOLOGY FOR ANALYSIS (FTA®) cards, such as FTA® Classic Cards, FTA® MINI CARDS, FTA® MICRO CARDS, and FTA® GENE CARDS (Whatman).

In some embodiments, the collected blood is stored prior to use. In one embodiment, the collected blood is stored at room temperature (i.e., approximately 22° C.). In another embodiment, the collected blood is stored at refrigerated temperatures, such as 4° C., prior to use. In some embodiments, a portion of the blood sample is used in accordance with the invention at a first instance of time whereas one or more remaining portions of the blood sample is stored for a period of time for later use. This period of time can be an hour or more, a day or more, a week or more, a month or more, a year or more, or indefinitely. For long term storage, storage methods well known in the art, such as storage at cryo temperatures (e.g below −60° C.) can be used. In some embodiments, in addition to storage of the blood or instead of storage of the blood, isolated genomic DNA is stored for a period of time for later use. Storage of such nucleic acids can be for an hour or more, a day or more, a week or more, a month or more, a year or more, or indefinitely.

In some embodiments of the present invention, blood cells are separated from whole blood collected from a subject using techniques known in the art. For example, blood collected from a subject can be subjected to Ficoll-Hypaque (Pharmacia) gradient centrifugation. Such centrifugation separates erythrocytes (red blood cells) from various types of nucleated cells and from plasma.

By way of example, but not limitation, macrophages can be obtained as follows. Mononuclear cells are isolated from peripheral blood of a subject, by syringe removal of blood followed by Ficoll-Hypaque gradient centrifugation. Tissue culture dishes are pre-coated with the subject's own serum or with AB+ human serum and incubated at 37° C. for one hour. Non-adherent cells are removed by pipetting. Cold (4° C.) 1 mM EDTA in phosphate-buffered saline is added to the adherent cells left in the dish and the dishes are left at room temperature for fifteen minutes. The cells are harvested, washed with RPMI buffer and suspended in RPMI buffer. Increased numbers of macrophages can be obtained by incubating at 37° C. with macrophage-colony stimulating factor (M-CSF). Antibodies against macrophage specific surface markers, such as Mac-1, can be labeled by conjugation of an affinity compound to such molecules to facilitate detection and separation of macrophages. Affinity compounds that can be used include but are not limited to biotin, photobiotin, fluorescein isothiocyante (FITC), or phycoerythrin (PE), or other compounds known in the art. Cells retaining labeled antibodies are then separated from cells that do not bind such antibodies by techniques known in the art such as, but not limited to, various cell sorting methods, affinity chromatography, and panning.

Blood cells can be sorted using a fluorescence activated cell sorter (FACS). Fluorescence activated cell sorting (FACS) is a known method for separating particles, including cells, based on the fluorescent properties of the particles. See, for example, Kamarch, 1987, Methods Enzymol 151:150-165. Laser excitation of fluorescent moieties in the individual particles results in a small electrical charge allowing electromagnetic separation of positive and negative particles from a mixture. An antibody or ligand used to detect a blood cell antigenic determinant present on the cell surface of particular blood cells is labeled with a fluorochrome, such as FITC or phycoerythrin. The cells are incubated with the fluorescently labeled antibody or ligand for a time period sufficient to allow the labeled antibody or ligand to bind to cells. The cells are processed through the cell sorter, allowing separation of the cells of interest from other cells. FACS sorted particles can be directly deposited into individual wells of microtiter plates to facilitate separation.

Magnetic beads can also be used to separate blood cells in some embodiments of the present invention. For example, blood cells can be sorted using a magnetic activated cell sorting (MACS) technique, a method for separating particles based on their ability to bind magnetic beads (0.5-100 m diameter). A variety of useful modifications can be performed on the magnetic microspheres, including covalent addition of an antibody which specifically recognizes a cell-solid phase surface molecule or hapten. A magnetic field is then applied, to physically manipulate the selected beads. In a specific embodiment, antibodies to a blood cell surface marker are coupled to magnetic beads. The beads are then mixed with the blood cell culture to allow binding. Cells are then passed through a magnetic field to separate out cells having the blood cell surface markers of interest. These cells can then be isolated.

In some embodiments, the surface of a culture dish may be coated with antibodies, and used to separate blood cells by a method called panning. Separate dishes can be coated with antibody specific to particular blood cells. Cells can be added first to a dish coated with blood cell specific antibodies of interest. After thorough rinsing, the cells left bound to the dish will be cells that express the blood cell markers of interest. Examples of cell surface antigenic determinants or markers include, but are not limited to, CD2 for T lymphocytes and natural killer cells, CD3 for T lymphocytes, CD11a for leukocytes, CD28 for T lymphocytes, CD19 for B lymphocytes,CD20 for B lymphocytes, CD21 for B lymphocytes, CD22 for B lymphocytes, CD23 for B lymphocytes, CD29 for leukocytes, CD14 for monocytes, CD41 for platelets, CD61 for platelets, CD66 for granulocytes, CD67 for granulocytes and CD68 for monocytes and macrophages.

A blood sample can be separated into cells types such as leukocytes, platelets, erythrocytes, etc. and such cell types can be used in accordance with the invention. Leukocytes can be further separated into granulocytes and agranulocytes using standard techniques and such cells can be used in accordance with the methods of the invention. Granulocytes can be separated into cell types such as neutrophils, eosinophils, and basophils using standard techniques and such cells can be used in accordance with the methods of the invention. Agranulocytes can be separated into lymphocytes (e.g., T lymphocytes and B lymphocytes) and monocytes using standard techniques and such cells can be used in accordance with the methods of the invention. T lymphocytes can be separated from B lymphocytes and helper T cells separated from cytotoxic T cells using standard techniques and such cells can be used in accordance with the methods of the invention. Separated blood cells (e.g., leukocytes) can be frozen by standard techniques prior to use in the present methods.

In some embodiments, blood cells are immortalized and/or proliferated in cell culture prior to use or storage. Any technique known in the art for immortalizing and/or proliferating blood cells can be used in accordance with the invention. In certain embodiments, the blood cells (e.g., lymphocytes) are infected with a virus, such as HTLV-I or HTLV-II, that immortalizes the cells. In other embodiments, the blood cells are transformed with an oncogene, such as bcl-2, that immortalizes the cells. In some embodiments, the blood cells are stored prior to or after proliferation and/or immortalization. In one embodiment, the blood cells are stored at cryo temperatures (e.g. below −60° C.).

In an embodiment, the biological sample collected from each subject is a swab of buccal cells from a subject's inner cheek (i.e., a cheek or buccal swab). In another embodiment, the biological sample is a tissue sample that comprises nucleated cells. In a particular embodiment, the tissue sample is breast, colon, lung, liver, ovarian, pancreatic, prostate, renal, bone or skin tissue. In a specific embodiment, the tissue sample is a biopsy.

In some embodiments, the collected cheek swab or tissue sample is stored prior to use. In one embodiment, the collected cheek swab or tissue sample is stored at room temperature (e.g., approximately 22° C.). In another embodiment, the collected cheek swab or tissue sample is stored at refrigerated temperatures, such as 4° C., prior to use. In some embodiments, a portion of the tissue sample is used in accordance with the invention at a first instance of time whereas one or more remaining portions of the tissue sample is stored for a period of time for later use. This period of time can be an hour or more, a day or more, a week or more, a month or more, a year or more, or indefinitely.

For long term storage, storage methods well known in the art, such as storage at cryo temperatures (e.g. below −60° C.) can be used. In some embodiments, in addition to storage of the cheek swab or tissue sample, or instead of storage of the cheek swab or tissue sample, isolated nucleic acids (e.g., isolated genomic DNA) is stored for a period of time for later use. Storage of such nucleic acids can be for an hour or more, a day or more, a week or more, a month or more, a year or more, or indefinitely.

A tissue sample can be separated into cells types such as epithelial cells, fibroblasts, etc. and such cell types can be used in accordance with the invention. In some embodiments, cells are immortalized and/or proliferated in cell culture prior to use or storage. Any technique known in the art for immortalizing and/or proliferating cells can be used in accordance with the invention. In certain embodiments, the cells (e.g., lymphocytes) are infected with a virus that immortalizes the cells. In other embodiments, the cells are transformed with an oncogene, such as bcl-2, that immortalizes the cells. In some embodiments, the cells isolated from a cheek swab or tissue sample are stored prior to or after proliferation and/or immortalization. In one embodiment, the cells are stored at cryo temperatures (e.g. below −60° C.).

The amount of a biological sample taken from the subject will vary according to the type of biological sample and the genotyping and/or sequencing method employed.

For example, the amount of blood collected will vary depending upon the site of collection, the amount required for genotyping and/or sequencing, and the comfort of the subject. In one embodiment, the amount of blood required is so small that more invasive procedures are not required to obtain the sample. For example, in some embodiments, all that is required is a drop of blood. This drop of blood can be obtained, for example, from a simple pinprick. In some embodiments, any amount of blood is collected that is sufficient to perform genotyping techniques and/or sequencing of genomic DNA. In certain embodiments, the amount of blood that is collected is 0.001 ml, 0.005 ml, 0.01 ml, 0.025 ml, 0.05 ml, 0.1 ml, 0.125 ml, 0.15 ml, 0.2 ml, 0.225 ml, 0.25 ml, 0.5 ml, 0.75 ml, 1 ml, 1.5 ml, 2 ml, 3 ml, 4 ml, 5 ml, 10 ml, 15 ml, 20 ml, 25 ml, 30 ml or more of blood is collected from a subject. In a specific embodiment, 0.001 ml to 30 ml, 0.01 to 25 ml, 0.01 to 20 ml, 0.01 ml to 10 ml, 0.1 ml to 30 ml, 0.1 to 25 ml, 0.1 to 20 ml, 0.1 ml to 10 ml, 0.1 ml to 5 ml, 1 to 5 ml of blood is collected from a subject. In another embodiment, the biological sample is a tissue and the amount of tissue taken from the subject is less than 10 milligrams, less than 25 milligrams, less than 50 milligrams, less than 1 gram, less than 5 grams, less than 10 grams, less than 50 grams, or less than 100 grams. In certain embodiments, the amount of a biological sample collected is sufficient to immortalize cells contained in the biological sample.

5.4.2 Genotyping

5.4.2.1 Methods for Extracting Genomic DNA

There are several known methods for extracting genomic DNA from biological samples, any of which can be used in the present invention. One nonlimiting example follows. Between 60-80 mg of tissue is placed in a petri dish with culture media and the tissue is divided into two pieces. The tissue is placed into two sterile 15 ml tubes and centrifuged for two minutes at 4° C. at 1500 rpm. The supernatant is removed and washed twice with 1 ml 1×PBS or DNA-buffer. The supernatant is removed the pellet resuspended in 2.06 ml DNA-buffer. About 100 μl proteinase K (10 mg/ml) and 240 μl 10% SDS is added, and the solution is shaken gently before incubation overnight at 45° C. in a waterbath. If there are still some tissue pieces visible, proteinase K is added again, the solution shaken gently, and incubated for another 5 hr at 45° C. About 2.4 ml of phenol is then added and the solution is shaken by hand for 5-10 minutes before centrifugation at 3000 rpm for 5 minute at 10° C. The supernatant is pipetted into a new tube, 1.2 ml of phenol is added, 1.2 ml of chloroform/isoamyl alcohol (24:1) is added and then the solution is shaken by hand for 5-10 min before centrifugation at 3000 rpm for 5 minute at 10° C. The supernatant is pipetted into a new tube and 2.4 ml of chloroform/isoamyl alcohol (24:1) is added. The solution is shaken by hand for 5-10 minutes, and centrifuged at 3000 rpm for 5 minutes at 10° C. The supernatant is pipetted into a new tube, 25 μl of 3 M sodium acetate (pH 5.2) is added, 5 ml ethanol is added, and then the solution shaken gently until the DNA precipitates. A glass pipette is heated over a gas burner and the end bent to a hook. The DNA thread out is fished out of the solution using the hook and transferred to a new tube. The DNA is washed in 70% ethanol and dried in a speed vacuum. The DNA is dissolved in 0.5-1 ml sterile water overnight (or longer if necessary) at 4° C. on a rotating shaker.

5.4.2.2 Sources of Marker Data

Several forms of genetic markers that are used for genotyping are known in the art. A common genetic marker is a single nucleotide polymorphism (SNP). It has been estimated that SNPs occur approximately once every 600 base pairs in the genome. See, for example, Kruglyak and Nickerson, 2001, Nature Genetics 27, 235, which is hereby incorporated by reference herein in its entirety. The present invention contemplates the use of genotypic databases such as SNP databases as a source of genetic markers. Alleles making up blocks of such SNPs in close physical proximity are often correlated, resulting in reduced genetic variability and defining a limited number of “SNP haplotypes” each of which reflects descent from a single ancient ancestral chromosome. See, for example, Fullerton et al., 2000, Am. J. Hum. Genet. 67, 881, which is hereby incorporated by reference herein in its entirety. Such a haplotype structure is useful in selecting appropriate genetic variants for analysis. Patil et al. found that a very dense set of SNPs is required to capture all the common haplotype information. Once common haplotype information is available, it can be used to identify much smaller subsets of SNPs useful for comprehensive whole-genome studies. See Patil et al., 2001, Science 294, 1719-1723, which is hereby incorporated by reference herein in its entirety.

Other suitable sources of genetic markers include databases that have various types of gene expression data from platform types such as spotted microarray (microarray), high-density oligonucleotide array (HDA), hybridization filter (filter), serial analysis of gene expression (SAGE) data. Another example of a genetic database that can be used is a DNA methylation database. For details on a representative DNA methylation database, see Grunau et al., 2001, MethDB—a public database for DNA methylation data, Nucleic Acids Research 29, pp. 270-274, which is hereby incorporated by reference herein in its entirety. In some embodiments, the markers that are used in the systems in methods are mitochondrial variants, mitochondrial haplogroups, Y chromosome markers, and copy number polymorphisms.

In one embodiment of the present invention, markers are identified in any type of genetic database that tracks variations in the human genome. Information that is typically represented in such databases is a collection of loci within the human genome. For each locus, variation information is provided. Variation information is any type of genetic variation information. Representative genetic variation information includes, but is not limited to, single nucleotide polymorphisms, restriction fragment length polymorphisms, random amplified polymorphic DNA, amplified fragment length polymorphisms, microsatellite markers, short tandem repeats, mitochondrial variants, mitochondrial haplogroups, Y chromosome markers, and copy number polymorphisms.

One form of genetic marker that can be used is a restriction fragment length polymorphism (RFLP). RFLPs are the product of allelic differences between DNA restriction fragments caused by nucleotide sequence variability. As is well known to those of skill in the art, RFLPs are typically detected by extraction of genomic DNA and digestion with a restriction endonuclease. Generally, the resulting fragments are separated according to size and hybridized with a probe. Single copy probes are preferred. As a result, restriction fragments from homologous chromosomes are revealed. Differences in fragment size among alleles represent an RFLP. See, for example, Helentjaris et al., 1985, Plant Mol. Bio. 5:109-118; and U.S. Pat. No. 5,324,631, each of which is hereby incorporated by reference herein in its entirety.

Another form of genetic marker that can be used is random amplified polymorphic DNA (RAPD). The phrase “random amplified polymorphic DNA” or “RAPD” refers to the amplification product of the distance between DNA sequences homologous to a single oligonucleotide primer appearing on different sites on opposite strands of DNA. Mutations or rearrangements at or between binding sites will result in polymorphisms as detected by the presence or absence of amplification product. See, for example, Welsh and McClelland, 1990, Nucleic Acids Res. 18:7213-7218; Hu and Quiros, 1991, Plant Cell Rep. 10:505-511, each of which is hereby incorporated by reference herein in its entirety.

Yet another form of marker data that can be used for genotyping is an amplified fragment length polymorphism (AFLP). AFLP technology refers to a process that is designed to generate large numbers of randomly distributed molecular markers. See, for example, Vos, 1995, “AFLP: a new technique for DNA fingerprinting,” Nucleic Acids Research 23: 4407-4414, which is hereby incorporated by reference herein in its entirety.

Still another form of marker data that can be used is “simple sequence repeats” or “SSRs”. SSRs are di-, tri- or tetra-nucleotide tandem repeats within a genome. The repeat region can vary in length between genotypes while the DNA flanking the repeat is conserved such that the same primers will work in a plurality of genotypes. A polymorphism exists in which the genotypes represents pairs of repeats of different lengths between the two flanking conserved DNA sequences. See, for example, Akagi et al., 1996, Theor. Appl. Genet. 93, 1071-1077; Bligh et al., 1995, Euphytica 86:83-85; Struss etal., 1998, Theor. Appl. Genet. 97, 308-315; Wu etal., 1993, Mol. Gen. Genet. 241, 225-235; and U.S. Pat. No. 5,075,217, each of which is hereby incorporated by reference herein in its entirety. SSRs are also known as satellites or microsatellites.

As described above, many genetic markers suitable for use with the present invention are publicly available. Those skilled in the art can also readily prepare suitable markers. For molecular marker methods, see generally, “The DNA Revolution” by Andrew H. Paterson 1996 (Chapter 2) in: Genome Mapping in Plants (ed. Andrew H. Paterson) by Academic Press/R. G. Landis Company, Austin, Tex., pp. 7-21, which is hereby incorporated by reference herein in its entirety.

Another source of marker data is the HapMap project, which is a public database of common variation in the human genome that contains more than one million single nucleotide polymorphisms (SNPs) for which accurate and complete genotypes have been obtained in at least 269 DNA samples from four populations, including ten 500-kilobase regions in which essentially all information about common DNA variation has been extracted. These data document the generality of recombination hotspots, a block-like structure of linkage disequilibrium and low haplotype diversity, leading to substantial correlations of SNPs with many of their neighbors. See, for example, The International HapMap Consortium, 2005, Nature 437, 1299-1320; The International HapMap Consortium, 2003, Nature 426, 789-796; The International HapMap Consortium, 2004, Nature Reviews Genetics 5, 467-475; Thorisson et al., 2005, Genome Research 15:1591-1593, each of which is hereby incorporated by reference herein in its entirety.

5.5 Cellular Constituent Detection and Abundance Measurement Assays

Once a population in accordance with the present invention has been defined, a cellular constituent abundance assay is performed on biological samples collected from the population. In some embodiments, the purpose of this assay is to measure cellular constituent abundances in such biological samples. In some embodiments, the purpose of this assay is to measure the presence or absence of specific cellular constituents in such biological samples. In some instances, the biological samples used to confirm that the subjects are members of a population in accordance with the present invention, such as those described in Section 5.4.1, can be used for such assays. In some embodiments, biological samples described in Section 5.5.1 are used for such assays. Representative cellular constituent abundance assays that can be performed using such assays include, but are not limited to, polymerase chain reaction or a related amplification methods such as those described in Section 5.5.2, microarray based transcript assays such as those described in Section 5.5.3, other methods of transcriptional state measurements such as those described in Section 5.5.4, measurements of other aspects of the biological state such as those described in Section 5.5.5, measurement of the translational state such as those described in Section 5.5.6, or other types of cellular constituent abundance measurements such as those described in Section 5.5.7.

5.5.1 Biological Samples

Samples from a subject used in accordance with the methods of the invention for detecting and/or measuring the abundance of a cellular constituent include any type of biological sample obtained from a subject and samples derived from a biological sample. In certain embodiments, in addition to the biological sample itself or in addition to material derived from the biological sample such as cells, nucleic acids or proteins, the sample used in the methods of this invention comprises added water, salts, glycerin, glucose, an antimicrobial agent, paraffin, a chemical stabilizing agent, heparin, an anticoagulant, or a buffering agent. In certain embodiments, the biological sample is blood, serum, urine, interstitial fluid, cartilage or synovial fluid. In a specific embodiment, the sample is a blood or serum sample. In another embodiment, the sample is a tissue sample. In a particular embodiment, the tissue sample is breast, colon, lung, liver, ovarian, pancreatic, prostate, renal, bone or skin tissue. In a specific embodiment, the tissue sample is a biopsy. The amount of biological sample taken from the subject will vary according to the type of biological sample, the type of cellular constituent to be measured, and the method to be employed to measure the abundance of the cellular constituent. In another embodiment, the biological sample is a tissue and the amount of tissue taken from the subject is less than 10 milligrams, less than 25 milligrams, less than 50 milligrams, less than 1 gram, less than 5 grams, less than 10 grams, less than 50 grams, or less than 100 grams.

In accordance with the methods of the invention, a sample derived from a biological sample is one in which the biological sample has been subjected to one or more pretreatment steps prior to the detection and/or measurement of a cellular constituent in the sample. In certain embodiments, a biological fluid is pretreated by centrifugation, filtration, precipitation, dialysis, or chromatography, or by a combination of such pretreatment steps. In other embodiments, a tissue sample is pretreated by freezing, chemical fixation, paraffin embedding, dehydration, permeablization, or homogenization followed by centrifugation, filtration, precipitation, dialysis, or chromatography, or by a combination of such pretreatment steps. In certain embodiments, the sample is pretreated by adjusting the concentration of a cellular constituent (e.g., protein or nucleic acid) in the sample, by adjusting the pH or ionic strength of the sample, or by removing contaminating proteins, nucleic acids, lipids, or debris from the sample prior to the detection and/or determination of the amount of a cellular constituent in the sample according to the methods of this invention.

In some embodiments, the collected biological sample is stored prior to use. In one embodiment, the biological sample is stored at room temperature (e.g., approximately 22° C.). In another embodiment, the collected biological sample is stored at refrigerated temperatures, such as 4° C., prior to use. In some embodiments, a portion of the biological sample is used in accordance with the invention at a first instance of time whereas one or more remaining portions of the biological sample is stored for a period of time for later use. This period of time can be an hour or more, a day or more, a week or more, a month or more, a year or more, or indefinitely. For long term storage, storage methods well known in the art, such as storage at cryo temperatures (e.g. below −60° C.) can be used. In some embodiments, in addition to storage of the biological sample, or instead of storage of the biological sample, isolated cellular constituents, such as RNA and proteins, are stored for a period of time for later use. Storage of such constituents can be for an hour or more, a day or more, a week or more, a month or more, a year or more, or indefinitely.

A biological sample can be separated into cells types, such as blood cells, epithelial cells, fibroblasts, etc., and such cell types can be used in accordance with the invention. Any technique known to one of skill in the art or described herein (e.g., in Section 5.4.1) for separating or isolating cells can be used in accordance with the invention. In some embodiments, cells are immortalized and/or proliferated in cell culture prior to use or storage. Any technique known in the art for immortalizing and/or proliferating cells can be used in accordance with the invention. In certain embodiments, the cells (e.g., lymphocytes) are infected with a virus that immortalizes the cells. In other embodiments, the cells are transformed with an oncogene, such as bcl-2, that immortalizes the cells. In some embodiments, the cells are stored prior to or after proliferation and/or immortalization. In one embodiment, the cells are stored at cryo temperatures (e.g. below −60° C.).

The biological samples for use in the methods of this invention is a human subject, preferably a human subject that is a member of an index founder population. The subject from which a biological sample is obtained and utilized in accordance with the methods of this invention includes, without limitation, an asymptomatic subject, a subject manifesting or exhibiting 1, 2, 3, 4 or more symptoms of a disorder, a subject clinically diagnosed as having a disorder, a subject predisposed to a disorder, a subject suspected of having a disorder, a subject diagnosed as having a disorder, a subject undergoing therapy for a disorder, a subject that has been medically determined to be free of a disorder (e.g., following therapy for the disorder), a subject that is managing a disorder, or a subject that has not been diagnosed with a disorder.

5.5.2 Polymerase and Related Amplification Methods

In one embodiment, the presence or the amount of a gene product, which is a form of cellular constituent, is detected and/or measured by polymerase chain reaction (PCR) based techniques. PCR provides a method for rapidly amplifying a particular nucleic acid sequence by using multiple cycles of DNA replication catalyzed by a thermostable, DNA-dependent DNA polymerase to amplify the target sequence of interest. PCR is well known in the art. PCR, is performed as described in Mullis and Faloona, 1987, Methods Enzymol., 155:335. Additional techniques to quantitatively measure RNA expression include, but are not limited to, ligase chain reaction, Qbeta replicase (see, e.g., International Application No. PCT/US87/00880), isothermal amplification method (see, e.g., Walker et al. (1992) PNAS 89:382-396), strand displacement amplification (SDA), repair chain reaction, Asymmetric Quantitative PCR (see, e.g., U.S. Publication No. US200330134307A1) and the multiplex microsphere bead assay described in Fuja et al., 2004, Journal of Biotechnology 108:193-205.

PCR is performed using template DNA or cDNA (at least 1 fg; more usefully, 1-1000 ng) and at least 25 pmol of oligonucleotide primers. A typical reaction mixture includes: 2 μl of DNA, 25 pmol of oligonucleotide primer, 2.5 l of 10 M PCR buffer 1 (Perkin-Elmer, Foster City, Calif.), 0.4 μl of 1.25 M dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30° C. and 72° C. is used. Initial denaturation of the template molecules normally occurs at between 92° C. and 99° C. for four minutes, followed by 20-40 cycles consisting of denaturation (94-99° C. for 15 seconds to 1 minute), annealing (temperature determined as discussed above; 1-2 minutes), and extension (72° C. for 1 minute). The final extension step is generally carried out for four minutes at 72° C., and may be followed by an indefinite (0-24 hour) step at 4° C.

Reverse transcription of RNA followed by PCR (“RT-PCR”) can be used to quantitatively or semi-quantitatively measure the expression level of a gene product in a biological sample. Techniques for performing RT-PCR are well known in the art and there are commercially available kits such as Taqman (Perkin Elmer, Foster City, Calif.).

The level of expression of a gene product can be measured by amplifying RNA from a sample using transcription based amplification systems (TAS), including nucleic acid sequence amplification (NASBA) and 3SR. See, e.g., Kwoh et al. (1989) PNAS USA 86:1173; International Publication No. WO 88/10315; and U.S. Pat. No. 6,329,179. These amplification techniques involve annealing a primer that has target specific sequences. Following polymerization, DNA/RNA hybrids are digested with RNase H while double stranded DNA molecules are heat denatured again. In either case the single stranded DNA is made fully double stranded by addition of a second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once with a polymerase such as T7 or SP6. The resulting products, whether truncated or complete, indicate target specific sequences.

5.5.3. Transcript Assay Using Microarrays

The techniques described in this section are particularly useful for the determination of the expression state or the transcriptional state of a cell or cell type or any other cell sample by measuring or obtaining expression profiles. These techniques include the provision of polynucleotide probe arrays that can be used to provide determination of the expression levels of a plurality of genes. These techniques further provide methods for designing and making such polynucleotide probe arrays.

The expression level of a nucleotide sequence in a gene can be measured by any high throughput technique. However measured, the result is either the absolute or relative amounts of transcripts or response data, including but not limited to values representing abundances or abundance ratios. Preferably, measurement of the expression profile is made by hybridization to transcript arrays, which is described in this subsection. In one embodiment, “transcript arrays” or “profiling arrays” are used. Transcript arrays can be employed for analyzing the expression profile in a cell sample and especially for measuring the expression profile of a cell sample of a particular tissue type or developmental state or exposed to a drug of interest.

In one embodiment, an expression profile is obtained by hybridizing detectably labeled polynucleotides representing the nucleic acid sequences in mRNA transcripts present in a cell (e.g., fluorescently labeled cDNA synthesized from total cell mRNA) to a microarray. A microarray is an array of positionally-addressable binding (e.g., hybridization) sites on a support for representing many of the nucleic acid sequences in the genome of a cell, preferably most or almost all of the genes. Each of such binding sites consists of nucleic acid probe bound to the predetermined region on the support. Microarrays are reproducible, allowing multiple copies of a given array to be produced and compared with each other. Preferably, microarrays are made from materials that are stable under binding (e.g., nucleic acid hybridization) conditions. Preferably, a given binding site or unique set of binding sites in the microarray will specifically bind (e.g., hybridize) to a nucleic acid sequence in a single gene from a cell (e.g., to an exon of a specific mRNA or a specific cDNA derived therefrom).

The microarrays used can include one or more test probes, each of which has a nucleic acid sequence that is complementary to a subsequence of RNA or DNA to be detected. Each probe typically has a different nucleic acid sequence, and the position of each probe on the solid surface of the array is usually known. Indeed, the microarrays are preferably addressable arrays, more preferably positionally addressable arrays. Each probe of the array is preferably located at a known, predetermined position on the solid support so that the identity (e.g., the sequence) of each probe can be determined from its position on the array (e.g., on the support or surface). In some embodiments, the arrays are ordered arrays.

Preferably, the density of probes on a microarray or a set of microarrays is 100 different (e.g., non-identical) probes per 1 cm² or higher. More preferably, a microarray used in the methods of the invention will have at least 550 probes per 1 cm², at least 1,000 probes per 1 cm², at least 1,500 probes per 1 cm² or at least 4,000 probes per 1 cm². In a particularly preferred embodiment, the microarray is a high density array, preferably having a density of at least 2,500 different probes per 1 cm². The microarrays used in the invention therefore preferably contain at least 10, at least 100, at least 500, at least 1,000, at least 2,500, at least 5,000, at least 10,000, at least 15,000, at least 20,000, at least 25,000, at least 50,000 or at least 55,000 different (e.g., non-identical) probes.

In one embodiment, the microarray is an array (e.g., a matrix) in which each position represents a discrete binding site for a nucleic acid sequence of a transcript encoded by a gene (e.g., for an exon of an mRNA or a cDNA derived therefrom). The array of binding sites on a microarray contains sets of binding sites for a plurality of genes. For example, in various embodiments, the microarrays of the invention can comprise binding sites for products encoded by fewer than 5% of the genes in the human genome. Alternatively, the microarrays of the invention can have binding sites for the products encoded by at least 5%, at least 10%, at least 25%, at least 50%, at least 75%, at least 85%, at least 90%, at least 95%, at least 99% or 100% of the genes in the human genome. In other embodiments, the microarrays of the invention can having binding sites for products encoded by fewer than 50%, by at least 50%, by at least 75%, by at least 85%, by at least 90%, by at least 95%, by at least 99% or by 100% of the genes expressed by a cell of a human. The binding site can be a DNA or DNA analog to which a particular RNA can specifically hybridize. The DNA or DNA analog can be, e.g., a synthetic oligomer or a gene fragment, e.g corresponding to an exon.

In some embodiments, a gene or an exon in a gene is represented in the microarrays by a set of binding sites comprising probes with different polynucleotides that are complementary to different sequence segments of the gene or the exon. Such polynucleotides are preferably of the length of 15 to 200 bases, more preferably of the length of 20 to 100 bases, most preferably 40-60 bases. Each probe sequence may also comprise linker sequences in addition to the sequence that is complementary to its target sequence. As used herein, a linker sequence is a sequence between the sequence that is complementary to its target sequence and the surface of support. In some instances, a microarray comprises one probe specific to each target gene or gene fragment. However, if desired, a microarray may contain at least 2, 5, 10, 100, or 1000 or more probes specific to some target genes under study. For example, the microarray may contain probes tiled across the sequence of the longest mRNA isoform of a gene at single base steps.

In specific embodiments of the invention, when an exon has alternative spliced variants, a set of nucleic acid probes of successive overlapping sequences, e.g., tiled sequences, across the genomic region containing the longest variant of an exon can be included in the microarray. The set of nucleic acid probes can comprise successive overlapping sequences at steps of a predetermined base intervals, e.g. at steps of 1, 5, or 10 base intervals, span, or are tiled across, the mRNA containing the longest variant. Such sets of nucleic acid probes therefore can be used to scan the genomic region containing all variants of a gene to determine the expressed variant or variants of the gene. Alternatively or additionally, a set of nucleic acid probes comprising gene specific probes and/or variant junction probes can be included in the microarray.

In some cases, a gene is represented in the microarray by a probe comprising a nucleic acid that is complementary to a portion of the full length gene. In some instances, a gene is represented by a single binding site on the profiling arrays. In some instances, a gene is represented by one or more binding sites on the microarray, each of the binding sites comprising a probe with a nucleic acid sequence that is complementary to an RNA fragment that is a portion of the target gene. The lengths of such probes are normally between 15-600 bases, preferably between 20-200 bases, more preferably between 30-100 bases, and most preferably between 40-80 bases. A probe of length 40-80 allows more specific binding of the gene than a probe of shorter length, thereby increasing the specificity of the probe to the target gene.

It will be apparent to one skilled in the art that any of the probe schemes, supra, can be combined on the same microarray and/or on different microarray within the same set of microarrays so that a more accurate determination of the expression profile for a plurality of genes (or cellular constituents) can be accomplished. It will also be apparent to one skilled in the art that the different probe schemes can also be used for different levels of accuracies in profiling. For example, a microarray comprising a small set of probes for each gene may be used to determine the relevant genes and/or RNA splicing pathways under certain specific conditions. A microarray or microarray set comprising larger sets of probes for the genes that are of interest is then used to more accurately determine the gene expression profile under such specific conditions. Other microarray strategies that allow more advantageous use of different probe schemes are also encompassed by the present invention.

It will be appreciated that when cDNA complementary to the RNA of a cell is made and hybridized to a microarray under suitable hybridization conditions, the level of hybridization to the site in the array corresponding to a particular gene will reflect the prevalence in the cell of mRNA or mRNAs containing the mRNA transcribed from that gene. For example, when detectably labeled (e.g., with a fluorophore) cDNA complementary to the total cellular mRNA is hybridized to a microarray, the site on the array corresponding to a gene (e.g., capable of specifically binding the product or products of the gene expressing) that is not transcribed or is removed during RNA splicing in the cell will have little or no signal (e.g., fluorescent signal), and a gene for which the encoded mRNA expressing the gene is prevalent will have a relatively strong signal.

5.5.3.1 Preparing Probes for Microarrays

As noted above, the “probe” to which a particular polynucleotide molecule, such as an exon, specifically hybridizes according to the invention is a complementary polynucleotide sequence. Preferably one or more probes are selected for each target gene. For example, when a minimum number of probes are to be used for the detection of a gene, the probes normally comprise nucleotide sequences greater than 40 bases in length. Alternatively, when a large set of redundant probes is to be used for a gene, the probes normally comprise nucleic acid sequences of 40-60 bases. The probes can also comprise sequences complementary to full length exons. It will be understood that each probe sequence can also comprise linker sequences in addition to the sequence that is complementary to its target sequence.

The probes may comprise DNA or DNA “mimics” (e.g., derivatives and analogs) corresponding to a portion of each gene in the human genome. In one embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates. DNA can be obtained, e.g., by polymerase chain reaction (PCR) amplification of gene segments from genomic DNA, cDNA (e.g., by RT-PCR), or cloned sequences. PCR primers are preferably chosen based on known sequence of the genes or cDNA that result in amplification of unique fragments (e.g., fragments that do not share more than 10 bases of contiguous identical sequence with any other fragment on the microarray). Computer programs that are well known in the art are useful in the design of primers with the required specificity and optimal amplification properties, such as Oligo version 5.0 (National Biosciences). Typically each probe on the microarray will be between 20 bases and 600 bases, and usually between 30 and 200 bases in length. PCR methods are well known in the art, and are described, for example, in Innis et al., eds., 1990, PCR Protocols: A Guide to Methods and Applications, Academic Press Inc., San Diego, Calif. It will be apparent to one skilled in the art that controlled robotic systems are useful for isolating and amplifying nucleic acids.

An alternative, preferred means for generating the nucleic acid probes of the microarray is by synthesis of synthetic polynucleotides or oligonucleotides, e.g., using N-phosphonate or phosphoramidite chemistries. See, for example, Froehler et al., 1986, Nucleic Acid Res. 14:5399-5407; and McBride et al., 1983, Tetrahedron Lett. 24:246-248, each of which is hereby incorporated by reference herein in its entirety. Synthetic sequences are typically between 15 and 600 bases in length, more typically between 20 and 100 bases, most preferably between 40 and 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid. See, e.g., Egholm et al., 1993, Nature 363:566-568; and U.S. Pat. No. 5,539,083, each of which is hereby incorporated herein by reference in its entirety. In alternative embodiments, the hybridization sites (e.g., the probes) are made from plasmid or phage clones of genes, cDNAs (e.g., expressed sequence tags), or inserts therefrom (Nguyen et al., 1995, Genomics 29:207-209).

5.5.3.2 Attaching Nucleic Acids to the Solid Surface

There are two main approaches used for microarray fabrication: deposition of nucleic acid fragments and in situ synthesis. The first type of fabrication involves two methods: deposition of PCR-amplified cDNA clones and printing of already synthesized nucleic acids. In situ manufacturing can be divided into photolithography, ink jet printing and electrochemical analysis. See for example, Draghici, 2003, Data Analysis Tools for DNA Microarrays, pp. 16-22, which is hereby incorporated by reference herein in its entirety.

In the deposition fabrication approach, microarray probes are attached to a solid support or surface, which may be made, e.g., from glass, plastic (e.g., polypropylene, nylon), polyacrylamide, nitrocellulose, gel, or other porous or nonporous material. One method for attaching the nucleic acids to a surface is by printing on glass plates, as is described generally by Schena et al., 1995, Science 270:467-470. This method is useful for preparing microarrays of cDNA (See also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

Another method for making microarrays is by making high-density polynucleotide arrays. Techniques are known for producing arrays containing thousands of nucleic acids complementary to defined sequences, at defined locations on a surface using photolithographic techniques for synthesis in situ (see, Fodor et al., 1991, Science 251:767-773; Lockhart et al., 1996, Nature Biotechnology 14:1675; U.S. Pat. Nos. 5,578,832; 5,556,752; and 5,510,270) or other methods for rapid synthesis and deposition of defined nucleic acids (Blanchard et al., Biosensors & Bioelectronics 11:687-690). When these methods are used, nucleic acids (e.g., 60-mers) of known sequence are synthesized directly on a surface such as a derivatized glass slide. The array produced can be redundant, with several polynucleic acid molecules per exon.

Other methods for making microarrays, e.g., by masking (Maskos and Southern, 1992, Nucl. Acids. Res. 20:1679-1684), can also be used. In principle, and as noted supra, any type of array, for example, dot blots on a nylon hybridization membrane (see Sambrook et al., supra) could be used. However, as will be recognized by those skilled in the art, very small arrays will frequently be preferred because hybridization volumes will be smaller.

In one embodiment, a microarray is manufactured by means of an ink jet printing device for nucleic acid synthesis, e.g., using the methods and systems described by Blanchard in International Patent Publication No. WO 98/41531, published Sep. 24, 1998; Blanchard et al., 1996, Biosensors and Bioelectronics 11:687-690; Blanchard, 1998, in Synthetic DNA Arrays in Genetic Engineering 20, Setlow, Ed., Plenum Press, New York at pages 111-123; and U.S. Pat. No. 6,028,189 to Blanchard. Specifically, the nucleic acid probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (e.g., the different probes). Polynucleic acid probes are normally attached to the surface covalently at the 3′ end of the polynucleic acid. Alternatively, polynuleic acid probes can be attached to the surface covalently at the 5′ end of the nucleic acid (see for example, Blanchard, 1998, in Synthetic DNA Arrays in Genetic Engineering 20, Setlow, Ed., Plenum Press, New York at pages 111-123).

5.5.3.3 Target Polynucleotide Molecules

Target polynucleotides that can be measured to obtain cellular constituent abundance data include, but are not limited to, RNA molecules such as, but by no means limited to, messenger RNA (mRNA) molecules, ribosomal RNA (rRNA) molecules, cRNA molecules (e.g., RNA molecules prepared from cDNA molecules that are transcribed in vivo) and fragments thereof. Other target polynucleotides which may also be measured to obtain cellular constituent abundance data include, but are not limited to DNA molecules such as genomic DNA molecules, cDNA molecules, and fragments thereof including oligonucleotides, ESTs, STSs, etc.

The target polynucleic acids can be from any source. For example, the target polynucleic acids may be naturally occurring nucleic acid molecules such as genomic or extragenomic DNA molecules isolated from a human, or RNA molecules, such as mRNA molecules, isolated from a human. Alternatively, the polynucleic acids may be synthesized, including, e.g., nucleic acid molecules synthesized enzymatically in vivo or in vitro, such as cDNA molecules, or polynucleotide molecules synthesized by PCR, RNA molecules synthesized by in vitro transcription, etc. The sample of target polynucleic acids can comprise, e.g., molecules of DNA, RNA, or copolymers of DNA and RNA. In preferred embodiments, the target polynucleic acids of the invention will correspond to particular genes or to particular gene transcripts (e.g., to particular mRNA sequences expressed in cells or to particular cDNA sequences derived from such mRNA sequences). However, in many embodiments, particularly those embodiments in which the polynucleic acids are derived from mammalian cells, the target polynucleic acids may correspond to particular fragments of a gene transcript. For example, the target polynucleic acids may correspond to different variants of the same gene, e.g., so that different splice variants of that gene may be detected and/or analyzed.

In some embodiments, the target polynucleic acids to be measured to obtain cellular constituent abundance data are prepared in vitro from nucleic acids extracted from cells. For example, in one embodiment, RNA is extracted from cells (e.g., total cellular RNA, poly(A)⁺ messenger RNA, fraction thereof) and messenger RNA is purified from the total extracted RNA. Methods for preparing total and poly(A)⁺ RNA are well known in the art, and are described generally, e.g., in Sambrook et al., supra. In one embodiment, RNA is extracted from cells of the various types of interest in this invention using guanidinium thiocyanate lysis followed by CsCl centrifugation and an oligo dT purification (Chirgwin et al., 1979, Biochemistry 18:5294-5299). In another embodiment, RNA is extracted from cells using guanidinium thiocyanate lysis followed by purification on RNeasy columns (Qiagen). cDNA is then synthesized from the purified mRNA using, e.g., oligo-dT or random primers. In some embodiments, the target polynucleic acids are cRNA prepared from purified messenger RNA extracted from cells. As used herein, cRNA is defined here as RNA complementary to the source RNA. The extracted RNAs are amplified using a process in which doubled-stranded cDNAs are synthesized from the RNAs using a primer linked to an RNA polymerase promoter in a direction capable of directing transcription of anti-sense RNA. Anti-sense RNAs or cRNAs are then transcribed from the second strand of the double-stranded cDNAs using an RNA polymerase (see, e.g., U.S. Pat. Nos. 5,891,636, 5,716,785; 5,545,522 and 6,132,997; see also, U.S. Pat. No. 6,271,002, and U.S. Provisional Patent Application Ser. No. 60/253,641, filed on Nov. 28, 2000, by Ziman et al.). Both oligo-dT primers (U.S. Pat. Nos. 5,545,522 and 6,132,997) or random primers (U.S. Provisional Patent Application Ser. No. 60/253,641, filed on Nov. 28, 2000, by Ziman et al.) that contain an RNA polymerase promoter or complement thereof can be used. Preferably, the target polynucleic acids are short and/or fragmented polynucleic acids which are representative of the original nucleic acid population of the cell.

The target polynucleic acids to be measured to obtain cellular constituent abundance data are detectably labeled in some embodiments. For example, cDNA can be labeled directly, e.g., with nucleotide analogs, or indirectly, e.g., by making a second, labeled cDNA strand using the first strand as a template. Alternatively, the double-stranded cDNA can be transcribed into cRNA and labeled. Preferably, the detectable label is a fluorescent label, e.g., by incorporation of nucleotide analogs. Other labels suitable for use include, but are not limited to, biotin, imminobiotin, antigens, cofactors, dinitrophenol, lipoic acid, olefinic compounds, detectable polypeptides, electron rich molecules, enzymes capable of generating a detectable signal by action upon a substrate, and radioactive isotopes. Exemplary suitable radioactive isotopes include ³²P, ³⁵S, ¹⁴C, ¹⁵N and ¹²⁵I. Fluorescent molecules suitable for the present invention include, but are not limited to, fluorescein and its derivatives, rhodamine and its derivatives, texas red, 5′carboxy-fluorescein (“FMA”), 2′,7′dimethoxy-4′,5′-dichloro-6-carboxy-fluorescein (“JOE”), N,N,N′,N′- tetramethyl-6-carboxy-rhodamine (“TAMRA”), 6′ carboxy-X-rhodamine (“ROX”), HEX, TET, IRD40, and IRD41. Fluorescent molecules that are suitable for the invention further include: cyamine dyes, including by not limited to Cy3, Cy3.5 and Cy5; BODIPY dyes including but not limited to BODIPY-FL, BODIPY-TR, BODIPY-TMR, BODIPY-630/650, and BODIPY-650/670; and ALEXA dyes, including but not limited to ALEXA-488, ALEXA-532, ALEXA-546, ALEXA-568, and ALEXA-594; as well as other fluorescent dyes which will be known to those who are skilled in the art. Electron rich indicator molecules suitable for the present invention include, but are not limited to, ferritin, hemocyanin, and colloidal gold. Alternatively, in some embodiments the target polynucleic acids may be labeled by specifically complexing a first group to the polynucleic acid. A second group, covalently linked to an indicator molecules and which has an affinity for the first group, can be used to indirectly detect the target polynucleic acid. In such an embodiment, compounds suitable for use as a first group include, but are not limited to, biotin and iminobiotin. Compounds suitable for use as a second group include, but are not limited to, avidin and streptavidin.

5.5.3.4 Hybridization to Microarrays

As described supra, nucleic acid hybridization and wash conditions are chosen so that the polynucleic acid molecules to be measured, in order to obtain cellular constituent abundance data, specifically bind or specifically hybridize to the complementary polynucleic acid sequences of the array, preferably to a specific array site in which its complementary DNA is located.

Microarrays containing double-stranded probe DNA situated thereon are preferably subjected to denaturing conditions to render the DNA single-stranded prior to contacting with the target polynucleic acids. Microarrays containing single-stranded nucleic acid probes (e.g., synthetic oligodeoxyribonucleic acids) may need to be denatured prior to contacting with the target polynucleic acids, e.g., to remove hairpins or dimers which form due to self complementary sequences.

Optimal hybridization conditions will depend on the length (e.g., oligomer versus polynucleic acid greater than 200 bases) and type (e.g., RNA, or DNA) of probe and target nucleic acids. As used herein, a polynucleic acid is any nucleic acid that is two base pairs long or longer (e.g., greater than 5 base pairs, greater than 10 base pairs, greater than 30 base pairs, greater than 50 base pairs, greater than 80 base pairs, etc.). General parameters for specific (e.g., stringent) hybridization conditions for polynucleic acids are described in Sambrook et al., (supra), and in Ausubel et al., 1987, Current Protocols in Molecular Biology, Greene Publishing and Wiley-Interscience, New York. When the cDNA microarrays of Schena et al. are used, typical hybridization conditions are hybridization in 5×SSC plus 0.2 percent SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Shena et al., 1996, Proc. Natl. Acad. Sci. U.S.A. 93:10614). Useful hybridization conditions are also provided in, e.g., Tijessen, 1993, Hybridization With Nucleic Acid Probes, Elsevier Science Publishers B.V. and Kricka, 1992, Nonisotopic DNA Probe Techniques, Academic Press, San Diego, Calif.

Particularly preferred hybridization conditions for use with the screening and/or signaling chips of the present invention include hybridization at a temperature at or near the mean melting temperature of the probes (e.g., within 5° C., more preferably within 2° C.) in 1 M NaCl, 50 mM MES buffer (pH 6.5), 0.5 percent sodium Sarcosine and thirty percent formamide.

5.5.3.5 Signal Detection and Data Analysis

It will be appreciated that when target sequences, e.g., cDNA or cRNA, complementary to the RNA of a cell is made and hybridized to a microarray under suitable hybridization conditions, the level of hybridization to the site in the array corresponding to any particular gene will reflect the prevalence in the cell of mRNA or mRNAs transcribed from that gene. For example, when detectably labeled (e.g., with a fluorophore) cDNA complementary to the total cellular mRNA is hybridized to a microarray, the site on the array for a gene (e.g., capable of specifically binding the product or products of the gene expressing) that is not transcribed will have little or no signal (e.g., fluorescent signal), and a gene for which the encoded mRNA expressing the gene is prevalent will have a relatively strong signal.

When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. In a some embodiment, the arrays are scanned with a laser fluorescence scanner with a computer controlled X-Y stage and a microscope objective. Excitation of the fluorophores is achieved with a laser, and the emitted light is detected with a photomultiplier tube. Such fluorescence laser scanning devices are described, e.g., in Schena et al., 1996, Genome Res. 6:639-645. Alternatively, the fiber-optic bundle described by Ferguson et al., 1996, Nature Biotech. 14:1681-1684, may be used to monitor mRNA abundance levels at a large number of sites simultaneously.

Signals are recorded and, in a preferred embodiment, analyzed by computer. In one embodiment, the scanned image is despeckled using a graphics program (e.g., Hijaak Graphics Suite) and then analyzed using an image gridding program that creates a spreadsheet of the average hybridization at each wavelength at each site. More information on image processing of microarrays is provided in Draghici, 2003, Data Analysis Tools for DNA Microarrays, pp. 33-59, hereby incorporated by reference herein in its entirety, including spot finding, image segmentation, quantification, and spot quality assessment.

5.5.4 Other Methods of Transcriptional State Measurements

The transcriptional state of a cell can be measured by other gene expression technologies known in the art. Several such technologies produce pools of restriction fragments of limited complexity for electrophoretic analysis, such as methods combining double restriction enzyme digestion with phasing primers (see, e.g., European Patent O 534858 A1, filed Sep. 24, 1992, by Zabeau et al.), or methods selecting restriction fragments with sites closest to a defined mRNA end (see, e.g., Prashar et al., 1996, Proc. Natl. Acad. Sci. USA 93:659-663). Other methods statistically sample cDNA pools, such as by sequencing sufficient bases (e.g., 20-50 bases) in each of multiple cDNAs to identify each cDNA, or by sequencing short tags (e.g., 9-10 bases) that are generated at known positions relative to a defined mRNA end (see, e.g., Velculescu, 1995, Science 270, 484-487, which is hereby incorporated by reference in its entirety).

5.5.5 Measurement of Other Aspects of the Biological State

In various embodiments of the present invention, aspects of the biological state other than the transcriptional state, such as the translational state, the activity state, or mixed aspects can be measured. Thus, in such embodiments, gene expression data can include translational state measurements or even protein expression measurements. Details of embodiments in which aspects of the biological state other than the transcriptional state are described below.

5.5.6 Translational State Measurements

Measurement of the translational state can be performed according to several methods. For example, whole genome monitoring of protein (e.g., the “proteome,”) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to the action of a drug of interest. Methods for making monoclonal antibodies are well known (see, e.g., Harlow and Lane, 1988, Antibodies. A Laboratory Manual, Cold Spring Harbor, N.Y., which is incorporated in its entirety for all purposes). In one embodiment, monoclonal antibodies are raised against synthetic peptide fragments designed based on genomic sequences of the cell. With such an antibody array, proteins from the cell are contacted to the array and their binding is assayed with assays known in the art.

Alternatively, proteins can be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., 1990, Gel Electrophoresis of Proteins: A Practical Approach, IRL Press, New York; Shevchenko et al., 1996, Proc. Natl. Acad. Sci. USA 93:1440-1445; Sagliocco et al., 1996, Yeast 12:1519-1533; and Lander, 1996, Science 274:536-539, which is hereby incorporated by reference in its entirety. The resulting electropherograms can be analyzed by numerous techniques, including mass spectrometric techniques, Western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug, or in cells modified by, e.g., deletion or over-expression of a specific gene.

5.5.7 Other Types of Cellular Constituent Abundance Measurements

The methods of the invention are applicable to any cellular constituent that can be detected and/or quantifiably measured. For example, where activities of proteins can be measured, embodiments of this invention can use such measurements. Activity measurements can be performed by any functional, biochemical, or physical means appropriate to the particular activity being characterized. Where the activity involves a chemical transformation, the cellular protein can be contacted with the natural substrate(s), and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle control, performance of the function can be observed. However known and measured, the changes in protein activities form the response data analyzed by the foregoing methods of this invention.

In some embodiments of the present invention, cellular constituent measurements are derived from cellular phenotypic techniques. One such cellular phenotypic technique uses cell respiration as a universal reporter. In one embodiment, 96-well microtiter plate, in which each well contains its own unique chemistry is provided. Each unique chemistry is designed to test a particular phenotype. Cells from a human subject are pipetted into each well. If the cells exhibits the appropriate phenotype, they will respire and actively reduce a tetrazolium dye, forming a strong purple color. A weak phenotype results in a lighter color. No color means that the cells don't have the specific phenotype. Color changes can be recorded as often as several times each hour. During one incubation, more than 5,000 phenotypes can be tested. See, for example, Bochner et al., 2001, Genome Research 11, p. 1246.

In some embodiments of the present invention, cellular constituent measurements are derived from cellular phenotypic techniques. One such cellular phenotypic technique uses cell respiration as a universal reporter. In one embodiment, 96-well microtiter plates, in which each well contains its own unique chemistry is provided. Each unique chemistry is designed to test a particular phenotype. Cells from the human 46 (FIG. 1) of interest are pipetted into each well. If the cells exhibit the appropriate phenotype, they will respire and actively reduce a tetrazolium dye, forming a strong purple color. A weak phenotype results in a lighter color. No color means that the cells don't have the specific phenotype. Color changes may be recorded as often as several times each hour. During one incubation, more than 5,000 phenotypes can be tested. See, for example, Bochner et al., 2001, Genome Research 11, 1246-55.

In some embodiments of the present invention, the cellular constituents that are measured are metabolites. Metabolites include, but are not limited to, amino acids, metals, soluble sugars, sugar phosphates, and complex carbohydrates. Such metabolites can be measured, for example, at the whole-cell level using methods such as pyrolysis mass spectrometry (Irwin, 1982, Analytical Pyrolysis: A Comprehensive Guide, Marcel Dekker, New York; Meuzelaar et al., 1982, Pyrolysis Mass Spectrometry of Recent and Fossil Biomaterials, Elsevier, Amsterdam), fourier-transform infrared spectrometry (Griffiths and de Haseth, 1986, Fourier transform infrared spectrometry, John Wiley, New York; Helm et al., 1991, J. Gen. Microbiol. 137, 69-79; Naumann et al., 1991, Nature 351, 81-82; Naumann et al., 1991, In: Modern techniques for rapid microbiological analysis, 43-96, Nelson, W. H., ed., VCH Publishers, New York), Raman spectrometry, gas chromatography-mass spectroscopy (GC-MS) (Fiehn et al., 2000, Nature Biotechnology 18, 1157-1161, capillary electrophoresis (CE)/MS, high pressure liquid chromatography/mass spectroscopy (HPLC/MS), as well as liquid chromatography (LC)-Electrospray and cap-LC-tandem-electrospray mass spectrometries. Such methods can be combined with established chemometric methods that make use of artificial neural networks and genetic programming in order to discriminate between closely related samples.

5.6 Identification of Loci of Interest by Linkage Analysis

This section describes a number of standard quantitative trait locus (QTL) linkage analysis algorithms that can be used to associate genomic regions with quantitative traits. Such linkage analysis is also sometimes referred to as QTL analysis. See, for example, Lynch and Walsch, 1998, Genetics and Analysis of Quantitative Traits, Sinauer Associates, Sunderland, Massachusetts, which is hereby incorporated by reference herein in its entirety. The primary aim of linkage analysis is to determine whether there exist pieces of the genome that are passed down through each of several families with multiple afflicted humans in a pattern that is consistent with a particular inheritance model and that is unlikely to occur by chance alone. In other words, the purpose of these algorithms is to identify a linkage region (e.g., a QTL) for a phenotypic trait exhibited by one or more humans. A linkage region is a region of the human genome that is responsible for a percentage of variation in a phenotypic trait in humans.

The recombination fraction can be denoted by θ and is bounded between 0 and 0.5. If θ=0.5 for two loci, then alleles at the two loci are transmitted independently with half of the gametes being recombinant, for the two loci, and half parental. In this case, the loci are unlinked. If θ<0.5, then alleles are not transmitted independently, and the two loci are linked. The extreme scenario is when θ=0, so that the two loci are completely linked, and there will be no recombination between the two loci during meiosis, e.g. all gametes are parental. Linkage analysis tests whether a marker locus, of known location, is linked to a locus of unknown location, that influences the phenotype under study. In other words, a linkage region is identified by comparing genotypes of humans in a group to a phenotype exhibited by the group using pedigree data. The genotype of each human at each marker in a plurality of markers in a genetic map produced by marker genotypic data is compared to a given phenotype of each human. The genetic map is created by placing genetic markers in genetic (linear) map order so that the positional relationships between markers are understood. The information gained from knowing the relationships between markers that is provided by a marker map provides the setting for addressing the relationship between linkage region effect and the location of the linkage region.

In some embodiments of the present invention, linkage analysis is based on any of the linkage region detection methods disclosed or referenced in Lynch and Walsch, 1998, Genetics and Analysis of Quantitative Traits, Sinauer Associates, Inc., Sunderland, Mass.

5.6.1 Phenotypic Data Used

It will be appreciated that the present invention provides no limitation on the type of phenotypic data that can be used. The phenotypic data can, for example, represent a series of measurements for a quantifiable phenotypic trait in a collection of humans. Such quantifiable phenotypic traits can include, for example, quantitative manifestations of any of the factors used to define an index founder population described, for example, in Section 5.3.2. Such quantifiable phenotypic traits can also include, for example, measurements of cellular constituents from members of the index founder population that are measured using the techniques described in Section 5.5. In some embodiments, the phenotypic data can be in a binary form that tracks the absence or presence of some phenotypic trait. As an example, a “1” can indicate that a particular subject of the founder population possesses a given phenotypic trait and a “0” can indicate that a particular subject of the index founder population lacks the phenotypic trait. The phenotypic trait can be any form of biological data that is representative of the phenotype of each member of the founder population under study. In some embodiments, the phenotypic traits are quantified and are may be referred to as quantitative phenotypes.

5.6.2 Genotypic Data Used

In order to provide the necessary genotypic data for linkage analysis, members of the index founder population are genotyped. In some embodiments, the genotypic data obtained in Section 5.4.2 is sufficient for this purpose. In some embodiments, more extensive genotyping is performed. Genotypic information is obtained from polymorphisms at each marker in a set of markers. Such polymorphisms include, but are not limited to, single nucleotide polymorphisms, microsatellite markers, restriction fragment length polymorphisms, short tandem repeats, copy number polymorphisms, sequence length polymorphisms, and DNA methylation patterns.

Linkage analyses use the genetic map derived from marker genotypic data as the framework for location of QTL for any given quantitative trait. In some embodiments, the intervals that are defined by ordered pairs of markers are searched in increments (for example, 2 cM), and statistical methods are used to test whether a QTL is likely to be present at the location within the interval. In one embodiment, linkage analysis statistically tests for a single QTL at each increment across the ordered markers in a marker set. The results of the tests are expressed as lod scores, which compares the evaluation of the likelihood function under a null hypothesis (no QTL) with the alternative hypothesis (QTL at the testing position) for the purpose of locating probable QTL. More details on lod scores is found in Section 5.9, as well as in Lander and Schork, 1994, Science 265, p. 2037-2048, which is hereby incorporated by reference in its entirety. Interval mapping searches through ordered sets of genetic markers in a systematic, linear (one-dimensional) fashion, testing the same null hypothesis and using the same form of likelihood at each increment.

5.6.3 Model Free Versus Model Based Linkage Analysis

Linkage analyses can generally be divided into two classes: model-based linkage analysis and model-free linkage analysis. Model-based linkage analysis assumes a model for the mode of inheritance whereas model-free linkage analysis does not assume a mode of inheritance. Model-free linkage analyses are also known as allele-sharing methods and non-parametric linkage methods. Model-based linkage analyses are also known as “maximum likelihood” and “lod score” methods. Either form of linkage analysis can be used in the present invention. Model-based linkage analysis is most often used for dichotomous traits and requires assumptions for the trait model. These assumptions include the disease allele frequency and penetrance function. For a disease trait, particularly those of interest to public health, the true underlying model is complex and unknown, so that these procedures are not applicable. The other form of linkage analysis (model-free linkage analysis) makes use of allele-sharing. Allele-sharing methods rely on the idea that relatives with similar phenotypes should have similar genotypes at a marker locus if and only if the marker is linked to the locus of interest. Linkage analyses are able to localize the locus of interest to a specific region of a chromosome, and the scope of resolution is typically limited to no less than 5 cM or roughly 5000 kb. For more information on model-based and model-free linkage analysis, see Olson et al., 1999, Statistics in Medicine 18, p. 2961-2981; Lander and Schork 1994, Science 265, p. 2037; and Elston, 1998, Genetic Epidemiology 15, p. 565, each of which is hereby incorporated by reference, as well as the sections below.

5.6.4 Known Programs for Performing Linkage Analysis

Many known programs can be used to perform linkage analysis in accordance with this aspect of the invention. One such program is MapMaker/QTL, which is the companion program to MapMaker and is the original QTL mapping software. MapMaker/QTL analyzes F₂ or backcross data using standard interval mapping. Another such program is QTL Cartographer, which performs single-marker regression, interval mapping (Lander and Botstein, Id.), multiple interval mapping and composite interval mapping (Zeng, 1993, PNAS 90: 10972-10976; and Zeng, 1994, Genetics 136: 1457-1468). QTL Cartographer permits analysis from F₂ or backcross populations. QTL Cartographer is available from North Carolina State University. Another program that can be used to perform linkage analysis is Qgene, which performs QTL mapping by either single-marker regression or interval regression (Martinez and Cumow 1994 Heredity 73:198-206). Using Qgene, eleven different population types (all derived from inbreeding) can be analyzed. Yet another program that may be used to perform linkage analysis is Map Manager QT, which is a QTL mapping program (Manly and Olson, 1999, Mamm Genome 10: 327-334). Map Manager QT conducts single-marker regression analysis, regression-based simple interval mapping (Haley and Knott, 1992, Heredity 69, 315-324), composite interval mapping (Zeng 1993, PNAS 90: 10972-10976), and permutation tests. A description of Map Manager QT is provided by the reference Manly and Olson, 1999, Overview of QTL mapping software and introduction to Map Manager QT, Mammalian Genome 10: 327-334.

Yet another program that can be used to perform linkage analysis is MAPL, which performs linkage analysis by either interval mapping (Hayashi and Ukai, 1994, Theor. Appl. Genet. 87:1021-1027) or analysis of variance. MAPL is available from the Institute of Statistical Genetics on Internet (ISGI), Yasuo, UKAI.

Another program that can be used for linkage analysis is R/qtl. This program provides an interactive environment for mapping QTLs in experimental crosses. R/qtl makes uses of the hidden Markov model (HMM) technology for dealing with missing genotype data. R/qtl has implemented many HMM algorithms, with allowance for the presence of genotyping errors, for backcrosses, intercrosses, and phase-known four-way crosses. R/qtl includes facilities for estimating genetic maps, identifying genotyping errors, and performing single-QTL genome scans and two-QTL, two-dimensional genome scans, by interval mapping with Haley-Knott regression, and multiple imputation. R/qtl is available from Karl W. Broman, Johns Hopkins University.

Those of skill in the art will appreciate that there are several other programs and algorithms that can be used in the steps of the methods of the present invention where linkage analysis is needed, and all such programs and algorithms are within the scope of the present invention.

5.6.5 Model-Based Parametric Linkage Analysis

In model-based linkage analysis, (also termed “lod score” methods or parametric methods), the details of a traits mode of inheritance is being modeled. Typically, particular values of the allele frequencies and the penetrance function are specified.

5.6.6 Model-Free Nonparametric Linkage Analysis

Model-based linkage analysis (classical linkage analysis) calculates a lod score that represents the chance that a given locus in the genome is genetically linked to a trait, assuming a specific mode of inheritance for the trait. Namely the allele frequencies and penetrance values are included as parameters and are subsequently estimated. In the case of complex diseases, it is often difficult to model with any certainty all the causes of familial aggregation. In other words, when the trait exhibits non-Mendelian segregation it can be difficult to obtain reliable estimates of penetrance values, including phenocopy risks, and the allele frequency of the disease mutation. Indeed it can be the case that different mutations at different loci have different kinds of effect on susceptibility, some major and some minor, some dominant and some recessive. If different modes of transmission are operative in different families, or if different loci interact in the same family, then no one transmission model may be appropriate. It is conceivable that if the transmission model for a linkage analysis is specified incorrectly the results produced from it will not be valid nor interpretable.

As a result of the difficulties described above, a variety of methods have been developed to test for linkage without the need to specify values for the parameters defining the transmission model, and these methods are termed model-free linkage analyses (meaning that they can be applied without regard to the true transmission model). Such methods are based on the premise that relatives who are similar with respect to the phenotype of interest will be similar at a marker locus, sharing identical marker alleles, only if a locus underlying the phenotype is linked to the marker.

Model-free linkage analyses (allele-sharing methods) are not based on constructing a model, but rather on rejecting a model. Specifically, one tries to prove that the inheritance pattern of a chromosomal region is not consistent with random Mendelian segregation by showing that affected relatives inherit identical copies of the region more often than expected by chance. Affected relatives should show excess allele sharing in regions linked to the QTL even in the presence of incomplete penetrance, phenocopy, genetic heterogeneity, and high-frequency disease alleles.

5.6.6.1 Identical by Descent—Affected Pedigree Member (IBD-APM) Analysis/Outbred Population

In one embodiment, nonparametric linkage analysis involves studying affected relatives in an index founder population to see how often a particular copy of a chromosomal region is shared identical-by descent (IBD), that is, is inherited from a common ancestor within the pedigree. The frequency of IBD sharing at a locus can then be compared with random expectation. An identity-by-descent affected-pedigree-member (IBD-APM) statistic can be defined as:

$$T ( s ) = {\sum }_{i , j} {x}_{ij} ( s ) .$$

where xᵢⱼ(s) is the number of copies shared IBD at position s along a chromosome, and where the sum is taken over all distinct pairs (i,j) of affected members in a founding population. The results from multiple families can be combined in a weighted sum T(s). Assuming random segregation, T(s) tends to a normal distribution with a mean μ and a variance σ that can be calculated on the basis of the kinship coefficients of the relatives compared. See, for example, Blackwelder and Elston, 1985, Genet. Epidemiol. 2, p. 85; Whittemore and Halpern, 1994, Biometrics 50, p. 118; Weeks and Lange, 1988, Am. J. Hum. Genet. 42, p. 315; and Elston, 1998, Genetic Epidemiology 15, p. 565. Deviation from random segregation is detected when the statistic (T-μ)/σ exceeds a critical threshold. The techniques in this section typically use an outbred population.

5.6.6.2 Affected Sib Pair Analysis/Outbred Population

Affected sib pair analysis is one form of IBD-APM analysis (Section 5.6.7.1). For example, two sibs can show IBD sharing for zero, one, or two copies of any locus (with a 25%-50%-25% distribution expected under random segregation). If both parents are available, the data can be partitioned into separate IBD sharing for the maternal and paternal chromosome (zero or one copy, with a 50%-50% distribution expected under random segregation). In either case, excess allele sharing can be measured with a χ² test. In the ASP approach, a large number of small pedigrees (affected siblings and their parents) are used. DNA samples are collected from each human and genotyped using a large collection of markers (e.g., microsatellites, SNPs). Then a check for functional polymorphism is performed. See, for example, Suarez et al., 1978, Ann. Hum. Genet. 42, p. 87; Weitkamp, 1981, N. Engl. J. Med. 305, p. 1301; Knapp et al., 1994, Hum. Hered. 44, p. 37; Holmans, 1993, Am. J. Hum. Genet. 52, p. 362; Rich etal., 1991, Diabetologica 34, p. 350; Owerbach and Gabbay, 1994, Am. J. Hum. Genet. 54, p. 909; and Berrettini et al., Proc. Natl. Acad. Sci. USA 91, p. 5918, each of which is hereby incorporated by reference in its entirety. For more information on Sib pair analysis, see Hamer et al., 1993, Science 261, p. 321, which is hereby incorporated by reference in its entirety.

In some embodiments, ASP statistics that test whether affected siblings pairs have a mean proportion of marker genes identical-by-descent that is>0.50 were computed. See, for example, Blackwelder and Elston, 1985, Genet. Epidemiol. 2, p. 85, which is hereby incorporated by reference in its entirety. In some embodiments, such statistics are computed using the SIBPAL program of the SAGE package. See, for example, Tran et al. 1991, (SIB-PAL) Sib-pair linkage program (Elston, New Orleans), Version 2.5, which is hereby incorporated by reference in its entirety. These statistics are computed on all possible affected pairs. In some embodiments the number of degrees of freedom of the t test is set at the number of independent affected pairs (defined per sibship as the number of affected individuals minus 1) in the sample instead of the number of all possible pairs. See, for example, Suarez and Eerdewegh, 1984, Am. J. Med. Genet. 18, p. 135. The techniques in this section typically use an outbred population.

5.6.6.3 Identical by State-Affected Pedigree Member (IBS-APM) Analysis/Outbred Population

In some instances, it is not possible to tell whether two relatives inherited a chromosomal region IBD, but only whether they have the same alleles at genetic markers in the region, that is, are identical by state (IBS). IBD can be inferred from IBS when a dense collection of highly polymorphic markers has been examined, but the early stages of genetic analysis can involve sparser maps with less informative markers so that IBD status can not be determined exactly. Various methods are available to handle situations in which IBD cannot be inferred from IBS. One method infers IBD sharing on the basis of the marker data (expected identity by descent affected-pedigree-member; IBD-APM). See, for example, Suarez et al., 1978, Ann. Hum. Genet. 42, p. 87; and Amos et al., 1990, Am J. Hum. Genet. 47, p. 842, each of which is hereby incorporated by reference in its entirety. Another method uses a statistic that is based explicitly on IBS sharing (an IBS-APM method). See, for example, Weeks and Lange, 1988, Am J. Hum. Genet. 42, p. 315; Lange, 1986, Am. J. Hum. Genet. 39, p. 148; Jeunemaitre et al., 1992, Cell 71, p. 169; and Pericak-Vance et al., 1991, Am. J. Hum. Genet. 48, p. 1034, each of which is hereby incorporated by reference in its entirety.

In one embodiment the IBS-APM techniques of Weeks and Lange, 1988, Am J. Hum. Genet. 42, p. 315; and Weeks and Lange, 1992, Am. J. Hum. Genet. 50, p. 859 are used. Such techniques use marker information of affected individuals to test whether the affected persons within a pedigree are more similar to each other at the marker locus than would be expected by chance. In some embodiments, the marker similarity is measured in terms of identity by state. In some embodiments, the APM method uses a marker allele frequency weighting function, ƒ(p), where p is the allele frequency, and the APM test statistics are presented separately for each of three different weighting functions, ƒ(p)=1, ƒ(p)=1/\sqrt{p}, and ƒ(p)=1/p. Whereas the second and third functions render the sharing of a rare allele among affected persons a more significant event, the first weighting function uses the allele frequencies only in calculation of the expected degree of marker allele sharing. The third function, ƒ(p)=1/p, can lead (more frequently than the first two) to a non-normal distribution of the test statistic. The second function is a reasonable compromise for generating a normal distribution of the test statistic while incorporating an allele frequency function. In some instances, the APM test statistics are sensitive to marker locus and allele frequency misspecification. See, for example, Babron, et al, 1993, Genet. Epidemiol. 10, p. 389, which is hereby incorporated by reference in its entirety. In some embodiments, allele frequencies are estimated from the pedigree data using the method of Boehnke, 1991, Am J. Hum. Genet. 48, p. 22, or by studying alleles. See, also, for example, Berrettini et al., 1994, Proc. Natl. Acad. Sci. USA 91, p. 5918.

In some embodiments, the significance of the APM test statistics is calculated from the theoretical (normal) distribution of the statistic. In addition, numerous replicates (e.g., 10,000) of these data, assuming independent inheritance of marker alleles and disease (i.e., no linkage), are simulated to assess the probability of observing the actual results (or a more extreme statistic) by chance. This probability is the empirical P value. Each replicate is generated by simulating an unlinked marker segregating through the actual pedigrees. An APM statistic is generated by analyzing the simulated data set exactly as the actual data set is analyzed. The rank of the observed statistic in the distribution of the simulated statistics determines the empirical P value. The techniques in this section typically use an outbred population.

5.6.6.4 Quantitative Traits

Model-free linkage analysis can also be applied to quantitative traits. An approach proposed by Haseman and Elston, 1972, Behav. Genet 2, p. 3, is based on the notion that the phenotypic similarity between two relatives should be correlated with the number of alleles shared at a trait-causing locus. Formally, one performs regression analysis of the squared difference Δ² in a trait between two relatives and the number x of alleles shared IBD at a locus. The approach can be suitably generalized to other relatives (Blackwelder and Elston, 1982, Commun. Stat. Theor. Methods 11, p. 449) and multivariate phenotypes (Amos et al., 1986, Genet. Epidemiol. 3, p. 255). See also, Marsh et al., 1994, Science 264, p. 1152, and Morrison et al., 1994, Nature 367, p. 284; Amos, 1994, Am. J. Hum Genet. 54, p. 535; and Elston, Am J. Hum. Genet. 63, p. 931, each of which is hereby incorporated by reference in its entirety.

5.7 Association Analysis

This section describes a number of association tests that can be used in the present invention. Association studies can be done with the index founder populations of the present invention. For a description of association studies see, for example, Nepom and Ehrlich, 1991, Annu. Rev. Immunol. 9, p. 493; Strittmatter and Roses, 1996, Annu. Rev. Neurosci. 19, p. 53; Vooberg et al., 1994, Lancet 343, p. 1535; Zoller et al., Lancet 343, p. 1536; Bennet et al., 1995, Nature Genet. 9, p. 284; Grant et al., 1996, Nature Genet. 14, p. 205; and Smith et al., 1997, Science 277, p. 959, each of which is hereby incorporated by reference in its entirety. As such, association studies test whether a disease and an allele show correlated occurrence across the population, whereas linkage studies determine whether there is correlated transmission within pedigrees.

Whereas linkage analysis involves the pattern of transmission of gametes from one generation to the next, association is a property of the population of gametes. Association exists between alleles at two loci if the frequency, with which they occur within the same gamete, is different from the product of the allele frequencies. If this association occurs between two linked loci, then utilizing the association will allow for fine localization, since the strength of association is in large part due to historical recombinations rather than recombination within a few generations of a family. In the simplest scenario, association arises when a mutation, which causes disease, occurs at a locus at some time, tₒ. At that time, the disease mutation occurs on a specific genetic background composed of the alleles at all other loci; thus, the disease mutation is completely associated with the alleles of this background. As time progresses, recombination occurs between the disease locus and all other loci, causing the association to diminish. Loci that are closer to the disease locus will generally have higher levels of association, with association rapidly dropping off for markers further away. The reliance of association on evolutionary history can provide localization to a region as small as 50-75 kb. Association is also called linkage disequilibrium. Association (linkage disequilibrium) can exist between alleles at two loci without the loci being linked.

Two forms of association analysis are discussed in the sections below, population based association analysis and family based association analysis. More generally, those of skill in the art will appreciate that there are several different forms of association analysis, and all such forms of association analysis can be used in steps of the present invention that require the use of quantitative genetic analysis.

In some embodiments, whole genome association studies are performed in accordance with the present invention. Two methods can be used to perform whole-genome association studies, the “direct-study” approach and the “indirect-study” approach. In the direct-study approach, all common functional variants of a given gene are cataloged and tested directly to determine whether there is an increased prevalence (association) of a particular functional variant in affected individuals within the coding region of the given gene. The “indirect-study” approach uses a very dense marker map that is arrayed across both coding and noncoding regions. A dense panel of polymorphisms (e.g., SNPs) from such a map can be tested in controls to identify associations that narrowly locate the neighborhood of a susceptibility or resistance gene. This strategy is based on the hypothesis that each sequence variant that causes disease must have arisen in a particular individual at some time in the past, so the specific alleles for polymorphisms (haplotype) in the neighborhood of the altered gene in that individual can be inherited in all of his or her affected descendants. The presence of a recognizable ancestral haplotype therefore becomes an indicator of the disease-associated polymorphism. In actuality, some of the alleles will be in association while others will not due to recombination occurring between the mutation and other polymorphisms.

In the case where the testing is by association analysis, a genetic map is not required because the association test takes place between a single marker (or a number of markers that are physically very close to one another, .e.g., a haplotype) and the trait of interest. In such a case, knowledge about the markers positions relative to others in the genome is not required because each marker is tested by itself. While it may be true that haplotypes are more easily formed with pedigree data, such information is not necessary (it can be computationally derived by examining the extent of linkage disequilibrium in an outbred population, or it can be formed directly by special resequencing assays that can track phase).

5.7.1. Population-Based (Model-Free) Association Analysis

In population-based (model-free) association studies, allele frequencies in afflicted humans are contrasted with allele frequencies in control humans in order to determine if there is an association between a particular allele and a complex trait. Population-based association studies for dichotomous traits are also referred to as case-control studies. A case-control study is based on the comparison of unrelated affected and unaffected individuals from a population. An allele A at a gene of interest is said to be associated with the phenotype if it occurs at significantly higher frequency among affected compared with control individuals. Statistical significance can be tested by a number of methods, including, but not limited to, logistic regression. Association studies are discussed in Lander, 1996, Science 274, 536; Lander and Schork, 1994, Science 265, 2037; Risch and Merikangas, 1996, Science 273, 1516; and Collins et al., 1997, Science 278, 1533, each of which is hereby incorporated by reference in its entirety.

As is true for case-control studies generally, confounding is a problem for inferring a causal relationship between a disease and a measured risk factor using population-based association analysis. One approach to deal with confounding is the matched case-control design, where individual controls are matched to cases on potential confounding factors (for example, age and sex) and the matched pairs are then examined individually for the risk factor to see if it occurs more frequently in the case than in its matched control. In some embodiments, cases and controls are ethnically comparable. In other words, homogeneous and randomly mating populations are used in the association analysis. In some embodiments, the family-based association studies described below are used to minimize the effects of confounding due to genetically heterogeneous populations. See, for example, Risch, 2000, Nature 405, p. 847, which is hereby incorporated by reference in its entirety.

5.7.2 Family-Based Association Analysis

Family-based association analysis is used in some embodiments of the invention. In some embodiments, each affected human is matched with one or more unaffected siblings (see, for example, Curtis, 1997, Ann. Hum. Genet. 61, p. 319) or cousins (see, for example, Witte, et al., 1999, Am J. Epidemiol. 149, p. 693) within the founder population and analytical techniques for matched case-control studies is used to estimate effects and to test a hypothesis. See, for example, Breslow and Day, 1989, Statistical methods in cancer research I, The analysis of case-control studies 32, Lyon: IARC Scientific Publications, hereby incorporated by reference, for an example of such studies. The following subsections describe some forms of family-based association studies. Those of skill in the art will recognize that there are numerous forms of family-based association studies and all such methodologies can be used in the present invention.

5.7.2.1 Transmisson Disequilibrium Test

In some embodiments, the transmission disequilibrium test (TDT) is used. TDT considers parents who are heterozygous for an allele and evaluates the frequency with which that allele is transmitted to affected offspring. By restriction to heterozygous parents, the TDT differs from other model-free tests for association between specific alleles of a polymorphic marker and a disease locus. The parameters of that locus, genotypes of sampled individuals, linkage phase, and recombination frequency are not specified. Nevertheless, by considering only heterozygous parents, the TDT is specific for association between linked loci.

TDT is a test of linkage and association that is valid in heterogeneous populations. It was originally proposed for data consisting of families ascertained due to the presence of a diseased child. The genetic data consists of the marker genotypes for the parents and child. The TDT is based on transmissions, to the diseased child, from heterozygous parents, or parents whose genotypes consist of different alleles. In particular, consider a biallelic marker with alleles M₁ and M₂. The TDT counts the number of times, n₁₂, that M₁M₂ parents transmit marker allele M₁ to the diseased child and the number of times, n₂₁, that M₂ is transmitted. If the marker is not linked to (correlated with) the disease locus, i.e. θ=0.5, or if there is no association between M₁ and the disease mutation, then conditional on the number of heterozygous parents, and in the absence of segregation distortion, n₁₂ is distributed binomially: B(n₁₂+n₂₁, 0.5). The null hypothesis of no linkage or no association can be tested with the statistic

$${T}_{TDT} = \frac{{( {n}_{12} - {n}_{21} )}^{2}}{{n}_{12} + {n}_{21}}$$

with statistical significance level approximated using the χ² distribution with one df or computed exactly with the binomial distribution. When transmissions from more than one diseased child per family are included in the TDT statistic, the test is valid only as a test of linkage.

Several extensions of the TDT test have been proposed and all such extensions are within the scope of the present invention. See, for example, Mortin and Collins, 1998, Proc. Natl. Acad. Sci. USA 95, p. 11389; Terwilliger, 1995, Am J Hum Genet 56, p. 777. See also, for example, Mueller and Young, 1997, Emery's Elements of Medical Genetics, Kalow ed., p. 169-175, Churchill Livingstone, Edinburgh; Zhao et al., 1998, Am. J. Hum. Genet. 63, p. 225; Roses, 2000, Nature 405, p. 857; Spielman et al., 1993, Am J. Hum. Genet. 52, p. 506; and Ewens and Spielman; Am. H. Hum. Genet. 57, p. 455.

5.7.2.2 Sibship-Based Test

In some embodiments, the sibship-based test is used. See, for example, Wiley, 1998, Cur. Pharmaceut. Des. 4, p. 417; Blackstock and Weir, 1999, Trends Biotechnol. 17, p. 121; Kozian and Kirschbaum, 1999, Trends Biotechnol. 17, p. 73; Rockett et al., Xenobiotica 29, p. 655; Roses, 1994, J. Neuropathol. Exp. Neurol 53, p. 429; and Roses, 2000, Nature 405, p. 857.

5.8 Fine-Mapping

In some embodiments in accordance with the present invention, fine mapping of quantitative trait loci (QTL) in candidate chromosomal regions is achieved by a multi-marker linkage disequilibrium mapping method using a dense marker map. The method compares the expected co-variances between haplotype effects given a postulated QTL position to the co-variances that are found in the data. The expected co-variances between the haplotype effects are proportional to the probability that the QTL position is identical by descent (IBD) given the marker haplotype information, which is calculated using the gene dropping method. Such a multi-marker disequilibrium mapping method is more accurate than those from a single marker transmission disequilibrium test. A general approach for the fine mapping method using this algorithm is described found in Meuwissen and Goddard, 2000, Genetics 155:421-430, which is hereby incorporated herein by reference in its entirety.

In some embodiments in accordance with the present invention, fine scale mapping of genes affecting complex traits is accomplished by combining linkage and linkage-disequilibrium information. Linkage information refers to recombinations within the marker-genotyped generations and linkage disequilibrium to historical recombinations over the last 10 to 10,000 generations. The identity-by-descent (IBD) probabilities at the quantitative trait locus (QTL) between first generation haplotypes are obtained from the similarity of the marker alleles surrounding the QTL, whereas IBD probabilities at the QTL between later generation haplotypes are obtained by using the markers to trace the inheritance of the QTL. The variance explained by the QTL is estimated by residual maximum likelihood using the correlation structure defined by the IBD probabilities. Unlinked background genes are accounted for by fitting a polygenic variance component. This method is robust against multiple genes affecting the trait, multiple mutations at the QTL, and relatively low marker density. Details of the method are described in Meuwissen et al., 2002, Genetics 161: 373-379, which is hereby incorporated herein by reference in its entirety.

In some embodiments in accordance with the present invention, fine mapping can be achieved by examining the issue of population stratification in association mapping studies. In case-control studies of association, population subdivision or recent admixture of populations can lead to spurious associations between a phenotype and unlinked candidate loci. With a model of sampling from a structured population, it has been shown that if population stratification exists, mapping can be achieved using unlinked marker loci. A case-control study design using unrelated control individuals is one approach for association mapping, provided that marker loci unlinked to the candidate locus are included in the study in order to test for stratification. Guidelines for how many unlinked marker loci should be used may be found in Prichard and Rosenberg, 1999, Am. J Hum. Genet. 65:220-228, which is hereby incorporated herein by reference in its entirety.

In some embodiments in accordance with the present invention, a general coalescent framework using genotype data in linkage disequilibrium-based mapping studies may be used in fine mapping. This approach unifies two main goals of gene mapping that have generally been treated separately in the past: detecting association (e.g., significance testing) and estimating the location of the causative variation. In one embodiment, the inference is separated into two stages. First, Markov chain Monte Carlo is used to sample from the posterior distribution of coalescent genealogies of all the sampled chromosomes without regard to phenotype. Then, the likelihood of the phenotype data is estimated under various models for mutation and penetrance at an unobserved disease locus by averaging across genealogies. The essential signal that these models look for is that, in the presence of disease susceptibility variants in a region, there is nonrandom clustering of the chromosomes on the tree according to phenotype. The extent of non-random clustering is captured by the likelihood and can be used to construct significance tests or Bayesian posterior distributions for location. A novelty of the framework is that it can naturally accommodate quantitative data. Detailed applications of the method to simulated data and to data from a Mendelian locus and from a proposed complex trait locus is found in Zollner and Pritchard, 2005, Genetics 169:1071-1092, which is hereby incorporated herein by reference in its entirety.

5.9 Logarithm of the Odds Scores

Denoting the joint probability of inheriting all genotypes P(g), and the joint probability of all observed data x (trait and marker species) conditional on genotypes P(x|g), the likelihood L for a set of data is
L=ΣP(g)P(x|g)
where the summation is over all the possible joint genotypes g (trait and marker) for all pedigree members. What is unknown in this likelihood is the recombination fraction θ, on which P(g) depends.

The recombination fraction θ is the probability that two loci will recombine during meiosis. The recombination fraction θ is correlated with the distance between two loci. By definition, the genetic distance is defined to be infinity between the loci on different chromosomes (nonsyntenic loci), and for such unlinked loci, θ=0.5. For linked loci on the same chromosome (syntenic loci), θ<0.5, and the genetic distance is a monotonic function of θ. See, e.g., Ott, 1985, Analysis of Human Genetic Linkage, first edition, Baltimore, MD, John Hopkins University Press. The essence of linkage analysis described in Section 5.10, is to estimate the recombination fraction θ and to test whether θ=0.5. When the position of one locus in the genome is known, genetic linkage can be exploited to obtain an estimate of the chromosomal position of a second locus relative to the first locus. In the techniques described in Section 5.10, linkage analysis is used to map the unknown location of genes predisposing to various quantitative phenotypes relative to a large number of marker loci in a genetic map. In the ideal situation, where recombinant and nonrecombinant meioses can be counted unambiguously, θ is estimated by the frequency of recombinant meioses in a large sample of meioses. If two loci are linked, then the number of nonrecombinant meioses N is expected to be larger than the number of recombinant meioses R. The recombination fraction between the new locus and each marker can be estimated as:

$$\hat{θ} = \frac{R}{N + R}$$

The likelihood of interest is:
L=ΣP(g|θ)P(x|g)
and inferences are based about a test recombination fraction θ on the likelihood ratio Λ=L(θ)/L(½) or, equivalently, its logarithm.

Thus, in a typical clinical genetics study, the likelihood of the trait and a single marker is computed over one or more relevant pedigrees. This likelihood function L(θ) is a function of the recombination fraction θ between the trait (e.g., classical trait or quantitative trait) and the marker locus. The standardized loglikelihood Z(θ)=log₁₀[L(θ)/L(½)] is referred to as a lod score. Here, “lod” is an abbreviation for “logarithm of the odds.” A lod score permits visualization of linkage evidence. As a rule of thumb, in human studies, geneticists provisionally accept linkage if
Z(\hat{θ})≧3
at its maximum for θ on the interval [0,½], where θ represents the θ value corresponding to this maximum. Further, linkage is provisionally rejected at a particular θ if
Z(\hat{θ})≦−2.

However, for complex traits, other rules have been suggested. See, for example, Lander and Kruglyak, 1995, Nature Genetics 11, p. 241.

Acceptance and rejection are treated asymmetrically because, with 22 pairs of human autosomes, it is unlikely that a random marker even falls on the same chromosome as a trait locus. See Lange, 1997, Mathematical and Statistical Methods for Genetic Analysis, Springer-Verlag, New York; Olson, 1999, Tutorial in Biostatistics: Genetic Mapping of Complex Traits, Statistics in Medicine 18, 2961-2981, which is hereby incorporated by reference herein in its entirety.

When the value of L is large, the null hypothesis of no linkage, L(½), to a marker locus of known location can be rejected, and the relative location of the locus corresponding to the quantitative trait can be estimated by \hat{θ}. Therefore, lod scores provide a method to calculate linkage distances as well as to estimate the probability that two genes (and/or QTLs) are linked.

Those of skill in the art will appreciate that lod score interpretation may be species dependent. For example, methods for evaluation the lod score in mouse are different from that described in this section. However, methods for computing lod scores are known in the art and the method described in this section is only by way of illustration and not by limitation.

5.10 Use of Genetic Markers Identified

The genetic markers (e.g. QTL, genes, or genetic markers) identified utilizing the methods of the invention can be used in the field of predictive medicine. In one aspect of the present invention, the genetic markers can be utilized to determine whether an individual is afflicted with a disorder or is at risk of developing a disorder. For example, mutations in a gene can be assayed in a biological sample. Such assays can be used for prognostic or predictive purpose to thereby prophylactically treat an individual prior to the onset of a disorder.

In another aspect of the invention, the genetic markers can be used to select appropriate therapies to prevent, treat, manage or ameliorate a disorder or a symptom thereof for an individual based on the genotype of the individual (e.g., the genotype of the individual examined to determine the ability of the individual to respond to a particular agent) (referred to herein as “pharmacogenomics”). Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism.” These pharmacogenetic conditions can occur either as rare defects or as polymorphisms.

In yet another aspect of the invention, the genetic markers can be used to monitor the influence of a therapy in clinical trials.

5.11 Analytic Kit Implementation

In a preferred embodiment, the methods of this invention can be implemented by use of kits for associating a clinical parameter with one or more candidate chromosomal regions in the human genome. Such kits contain microarrays, such as those described in subsections below. The microarrays contained in such kits comprise a solid phase, e.g., a surface, to which probes are hybridized or bound at a known location of the solid phase. Preferably, these probes consist of nucleic acids of known, different sequence, with each nucleic acid being capable of hybridizing to an RNA species or to a cDNA species derived therefrom. In a particular embodiment, the probes contained in the kits of this invention are nucleic acids capable of hybridizing specifically to nucleic acid sequences derived from RNA species in cells collected from an human of interest.

Some embodiments of the present invention comprise a method of using a microarray, where the microarray comprises a plurality of probe spots, where at least twenty percent, at least thirty percent, at least forty percent, at least fifty percent, at least sixty percent, or at least seventy percent of the probe spots in the plurality of probe spots each comprise at least a hybridizable portion of the coding sequence of a gene that encompasses a marker in the chromosomal regions identified by any of the methods, computer programs products, or computer systems of the present invention. As used herein, the term “probe spot” is a discrete addressable location on a microarray that typically contains a probe. In the case of nucleic acid arrays, the probe is a single stranded nucleic acid that binds to a target nucleic acid under nucleic acid microarray hybridization conditions. In the case of protein arrays, the probe is a molecular entity such as a monoclonal antibody that binds to a target protein under protein microarray hybridization conditions. For more information on probes in the context of nucleic acid arrays, see Draghici, 2003, Data Analysis Tools for DNA Microarrays, chapter 2, which is hereby incorporated by reference herein in its entirety for such purpose.

In a preferred embodiment, a kit of the invention also contains one or more modules described in Section 5.1 in conjunction with FIGS. 1 and 2, encoded on computer readable medium, and/or an access authorization to use the databases described above from a remote networked computer.

In another preferred embodiment, a kit of the invention further contains software capable of being loaded into the memory of a computer system such as the one described supra, and illustrated in FIG. 1. The software contained in the kit of this invention, is essentially identical to the software described above in conjunction with FIG. 1.

Alternative kits for implementing the analytic methods of this invention will be apparent to one of skill in the art and are intended to be comprehended within the accompanying claims.

5.12 Exemplary Diseases

The present invention can be used to identify loci that are linked to complex traits in index founder populations. In some embodiments, the complex trait is a phenotype that does not exhibit Mendelian recessive or dominant inheritance attributable to a single gene locus. In some embodiments, the trait is adult macular degeneration, asthma, ataxia telangiectasia, autism, bipolar disorder, breast cancer, a cancer, cardiomyopathy, celiac disease, a Charcot-Marie-Tooth disease, colon cancer, a dementia, insulin-dependent diabetes mellitus, T2 diabetes, diabetic retinopathy, glaucoma, heart disease, hereditary early-onset Alzheimer's disease, early-onset Parkinson's disease, an epilepsy, familial hypercholesteremia, hereditary nonpolyposis, hypertension, infection, late-onset Alzheimer's disease, late-onset Parkinson's disease, a leukemia, longevity, lung cancer, maturity-onset diabetes of the young, mellitus, migraine, multiple sclerosis, myofibrillar myopathy, a neuropathy, nonalcoholic fatty liver (NAFL), nonalcoholic steatohepatitis (NASH), non-insulin-dependent diabetes mellitus (NIDDM), non-syndromic-blindness, non-syndromic deafness, osteoporosis, pancreatic diabetes, pancreatic cancer, Parkinsonisms, polycystic kidney disease, prostate cancer, psoriases, rheumatoid arthritis, schizophrenia, sickle cell disease, steatohepatitis, a stroke, systemic lupus erythematosus, or xeroderma pigmentosum.

5.13 Multivariate Statistical Models

Multivariate statistical techniques can be used to determine whether the genes identified in the methods of the present invention affect a particular clinical trait, such as a complex disease trait. The form of multivariate statistical analysis used in some embodiments of the present invention is dependent upon the type of genotypic data that is available. Methods described in Allison, 1998, Multiple Phenotype Modeling in Gene-Mapping Studies of Quantitative Traits: Power Advantages, Am J. Hum. Genetics 63, pp. 1190-1201, are used, including, but not limited to, those of Amos et al., 1990, Am J Hum. Genetics 47, pp. 247-254. Each of these references is hereby incorporated by reference in its entirety. In some embodiments, gene expression data is collected for multiple tissue types. In such instances, multivariate analysis can be used to determine the true nature of a complex disease.

5.14 Sequencing Methods

Any technique known to one of skill in the art may be used to sequence a nucleic acid. Sequencing techniques that can be used include the Maxam-Gilbert and Sanger sequencing techniques. Using the Maxam-Gilbert technique, DNA fragments of different lengths are produced using chemicals that cleave DNA. In the Sanger technique, DNA chains of varying lengths are produced using four different enzymatic reactions and a chemical is included to stop the DNA replication at positions occupied by one of the four bases. Both techniques use gel electrophoresis to separate DNA molecules that differ in length by only one nucleotide. See, e.g., Ausubel et al., eds., 1998, Current Protocols in Molecular Biology, John Wiley & Sons, Inc., New York.

5.15 Computer and Computer Program Product Implementations

The present invention can be implemented as a computer program product that comprises a computer program mechanism embedded in a computer readable storage medium. Further, any of the methods of the present invention can be implemented in one or more computers. Further still, any of the methods of the present invention can be implemented in one or more computer program products. Some embodiments of the present invention provide a computer program product that encodes any or all of the methods disclosed herein. Such methods can be stored on a CD-ROM, DVD, magnetic disk storage product, or any other computer readable data or program storage product. Such methods can also be embedded in permanent storage, such as ROM, one or more programmable chips, or one or more application specific integrated circuits (ASICs). Such permanent storage can be localized in a server, 802.11 access point, 802.11 wireless bridge/station, repeater, router, mobile phone, or other electronic devices. Such methods encoded in the computer program product can also be distributed electronically, via the Internet or otherwise, by transmission of a computer data signal (in which the software modules are embedded) either digitally or on a carrier wave.

Some embodiments of the present invention provide a computer program product that contains any or all of the program modules shown in FIG. 1. These program modules can be stored on a CD-ROM, DVD, magnetic disk storage product, or any other computer readable data or program storage product. The program modules can also be embedded in permanent storage, such as ROM, one or more programmable chips, or one or more application specific integrated circuits (ASICs). Such permanent storage can be localized in a server, 802.11 access point, 802.11 wireless bridge/station, repeater, router, mobile phone, or other electronic devices. The software modules in the computer program product can also be distributed electronically, via the Internet or otherwise, by transmission of a computer data signal (in which the software modules are embedded) either digitally or on a carrier wave.

5.16 Necessity and Sufficiency Genes

Index founder populations provide an opportunity to discover simple disease-causing (or preventing) genetic variations that are likely to be masked or obscured in non-index founder populations. Such genes are masked in non-index founder populations because of the much broader heterogeneity of disease, due to both genetic and non-genetic causes in non-index founder populations.

Specifically, two such classes of genes are defined: necessity genes and sufficiency genes. A “sufficiency” gene is a specific genetic variant that, in and of itself, is sufficient to cause disease. A “necessity” genetic variant is one that is absolutely required to cause disease, yet by itself, is not sufficient to cause disease. Similarly, it is expected that there may also exist resistance versions of both necessity and sufficiency genes. That is, some individuals might have genetic factors that can block certain diseases. There are several parallels and symmetries between the concepts of susceptibility and resistance, and also between necessity and sufficiency. This will become clear when the genetic concepts of recessive and dominant effects are introduced below.

Table 5, panels A-D, assume a 200 patient sample of 100 cases (D+) and 100 controls (D−). In panel 1 A, a disease sufficiency gene is assumed to cause 10% of cases, and this gene is dominant. That is, 10% of D+ individuals also have at least one copy (dominance) of this disease marker (M+). Importantly, by definition, none of the controls (D−) have any copies of the marker—they are 100% M−. Of course, in practice experimental error can occur, such as misclassification of cases or controls. However, as shown below, these concepts are relatively robust to such errors, even with relatively small sample sizes.

TABLE 5
Sufficiency and necessity gene examples

Panel A. “Sufficiency” gene

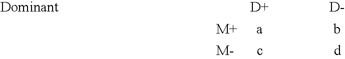

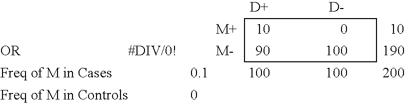

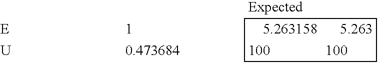

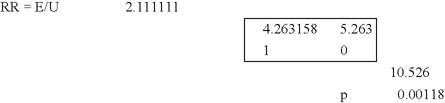

Panel B. “Necessity” gene
Dominant

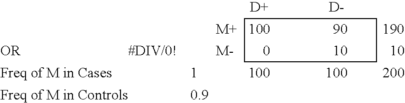

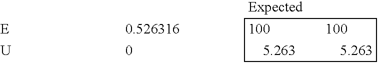

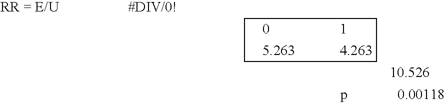

Panel C. “Sufficiency” gene
Recessive

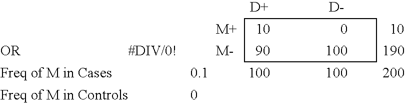

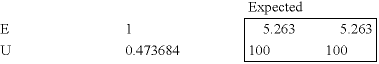

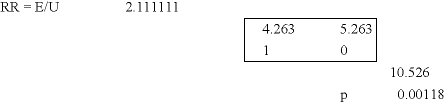

Panel D. “Necessity” gene
Recessive

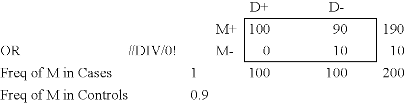

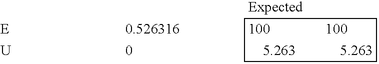

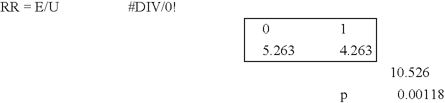

Note that in panel A, even this relatively small effect is detectable with a relatively small sample size (p = 0.0012). Note also, that if one were instead looking at a sufficiency gene for disease resistance with the same parameters and genetic characteristics, all one would need to do is switch the D+/D− column headings, leaving the rest of the table intact.

In fact, all of the actual calculations in Table 5 A-D are identical. This is done intentionally, so that one can focus on the symmetry of necessity and sufficiency, and to explain additional genetic nuances arising from each of the four illustrated examples. In Panel B, a dominant necessity gene causing disease is assumed. Even though the gene is very frequent, and found in 90% of controls, it would still be detectable with this sample size. Another interpretation of this result is that most of the population is genetically vulnerable to disease, except for the 10% of control individuals (D−) who are likewise M− Here lies the symmetry between necessity and sufficiency: if one variant in a gene is a dominant necessity gene for disease, the absence of this variant is sufficient for resistance. In genetic terms, the absence of a specific allele at an autosomal locus is equivalent to the presence of two copies of an alternative allele. That is, each alternative allele could be viewed as a recessive sufficiency allele for resistance. Even more than that, compound heterozygotes of such alleles would likewise be protective.

In panels C and D of Table 5, the recessive versions of sufficiency genes and necessity genes are illustrated, respectively. Although the mathematics is entirely identical to the dominant version of each gene, the difference lies in the interpretation of the M+/M− columns. That is, an individual with only one copy of a recessive sufficiency gene would be M−, since “M+” status requires two copies of the gene.

These considerations highlight some additional considerations derived from population genetics, as follows. Hardy-Weinberg Equilibrium (HWE) is the concept that under many common circumstances, a population's genotype frequency is predictable from its allele frequencies. Deviations from HWE are often used to suggest the action of other forces, and may also be used, in our examples, to detect and support the action of necessity and sufficiency genes. Actual detection will depend, among other things, on disease prevalence. Taking autism as our example, with a prevalence of 1% in some of the index founder populations, the example in panel A of Table 5 (a dominant sufficiency gene for disease) would have a tremendous deviation from HWE, since only 0.1% of the whole population (10% of 1%) should be heterozygous, yet the sample would show 10% of cases heterozygous versus 0% of controls.

Another important consideration for necessity and sufficiency genes is their hereditability. As sufficiency is defined herein, one expects to see essentially Mendelian inheritance. Whether dominant or recessive, sufficiency disease genes should show strictly Mendelian inheritance. Necessity disease genes, on the other hand, do not show Mendelian inheritance since one or more co-factors are necessary to cause disease. However, in this case the symmetry with sufficiency resistance genes mentioned above can be used: all alleles that are alternative to a dominant necessity disease gene are (at least) recessive sufficiency resistance genes. Furthermore, all allelic alternatives to a recessive necessity disease gene are in fact dominant sufficiency resistance genes, since any one of them should block disease.

Given the heritability considerations above, index founder population are an excellent resource for discovering Mendelian genes causing disease or disease resistance, even when the actual disease is much more complicated in general. This is especially true if the index founder population has a high degree of consanguinity, since even very rare recessive genetic factors can be exposed.

The above definitions and descriptions of necessity and sufficiency genes are very rigorous, and it is worthwhile to investigate how relaxing these restrictions affects their detectability. Fortunately, this is easily accomplished in a single, simple framework. Returning to the case-control scenario in Table 5, it is recognized that relaxing either the D+/D− dichotomy or the M+/M− dichotomy is tantamount to allowing a certain amount of misclassification. For instance, in panel A, if two of the 100 controls were either misclassified as M+ (or even if they were actually M+), the sufficiency gene would still be detectable (p=0.017). Thus, even though necessity and sufficiency are described rigorously and in absolute terms, in practice these concepts can tolerate some degree of exception and even experimental error.

### 6. EXAMPLE

The systems and methods of the present invention identify index founder populations. An exemplary implementation follows. Before step 202, a potential index founder population of the Wahhabi Sect of Qatar from Section 5.3.1.3 is selected. The systems and methods of the present invention then apply one or more filtering criteria as described in steps 202 to 206 in order to validate that the test population as an index founder population.

In accordance with step 202 data is received on the populations of Qatar. In 2005, there are approximately 860,000 people in Qatar. The Arabs of the Wahhabi Sect are a minority representing about 20% of the population of Qatar, while the remaining population is made up of other Arabs, Pakistanis, Indians, and Iranians. This means there are approximately 172,000 members in the Qatar Wahhabi Sect as a potential index founder population. Using Table 1 the consanguinity rates in Qatar are noted to exceed 50%. Using Table 3, above, an index rating of 1024 assists in validation of the Wahhabi Sect as an index founder population. In step 204, the population is further validated the by genotyping nine members from the Wahhabi Sect: {Genotypic Data: Member 1; Member 2; Member 3; Member 4; Member 5; Member 6; Member 7; Member 8; Member 9}. Using Table 3, the modality of consanguinity in the 9 member selection parents are First Cousin and the index rating system gives 512, thereby further validating the Wahhabi Sect as an index founder population. In accordance with step 206, one is able to add additional genotypic and /or phenotypic information. In this example, such additional data is not used.

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed:

1. A method of associating a clinical parameter with one or more candidate chromosomal regions in the human genome, said method comprising:
(A) identifying a first index founder population in a first test population based upon the genotype X of each member of said first test population, wherein the posterior probability Pr(K|X) for said first index founder population is greater for K=1 than any other integer K, where K is a number of subpopulations in said first index founder population;
(B) measuring said clinical parameter for each respective member of said first index founder population; and
(C) performing a quantitative phenotypic analysis between (i) the genotype X of each respective member of said first index founder population and (ii) the clinical parameter thereby identifying one or more candidate chromosomal regions in the human genome that associate with the clinical parameter.

2. The method of claim 1, wherein said quantitative phenotypic analysis is linkage analysis and wherein the method further comprises obtaining pedigree data for all or a portion of the first index founder population and wherein a chromosomal region in the one or more candidate chromosomal regions is a quantitative trait locus (QTL).

3. The method of claim 1, wherein said quantitative phenotypic analysis is association analysis and a chromosomal region in the one or more candidate chromosomal regions is a QTL.

4. The method of claim 1, the method further comprising:
(D) communicating the identity of the one or more chromosomal regions to a user, a display, an internal or external component of a computer, a remote computer, or to storage on a computer readable medium.

5. The method of claim 1, wherein said genotype X comprises at least five markers.

6. The method of claim 1, wherein said genotype X comprises at least one hundred markers.

7. The method of claim 1, wherein said genotype X comprises at least one thousand markers.

8. The method of claim 1, wherein said genotype X comprises at least twenty thousand markers.

9. The method of claim 1, wherein said genotype X comprises a haplotype.

10. The method of claim 1, wherein said clinical parameter is an abundance level measurement for a gene in a biological sample obtained from said respective member.

11. The method of claim 10, wherein said abundance level measurement for said gene is determined by measuring an amount of a cellular constituent for said gene in one or more cells in said biological sample.

12. The method of claim 11, wherein the amount of the cellular constituent for said gene comprises an abundance measurement of mRNA transcripts, cDNAs, or cRNAs for mRNA transcribed from the gene, or nucleic acid derived from any of the foregoing.

13. The method of claim 11, wherein the amount of the cellular constituent comprises an abundance of a protein encoded by the gene that are present in or secreted by one or more cells in said biological sample.

14. The method of claim 11, wherein said biological sample is obtained from a single tissue type or single organ type in said respective member.

15. The method of claim 1, wherein the clinical parameter is absence, presence, or stage of a disease.

16. The method of claim 15, wherein the disease is a complex disease.

17. The method of claim 1, wherein the posterior probability Pr(K|X) for said first index founder population for any K less than 6 and greater than 1 is 0.4 or less.

18. The method of claim 1, wherein the posterior probability Pr(K|X) for said first index founder population for any K less than 6 and greater than 1 is 0.3 or less.

19. The method of claim 1, the method further comprising, prior to said identifying step (A), determining that the consanguinity rate of the first test population is ten percent or greater.

20. The method of claim 1, the method further comprising, prior to said identifying step (A), determining that the consanguinity rate of the first test population is twenty percent or greater.

21. The method of claim 1, the method further comprising, prior to said identifying step (A), determining that the consanguinity rate of the first test population is forty percent or greater.

22. The method of claim 1, the method further comprising, prior to said identifying step (A), determining that the average coefficient of inbreeding F_{avg} in the first test population is 0.20 or greater.

23. The method of claim 1, the method further comprising, prior to said identifying step (A), identifying each member of the first test population using at least one criterion selected from the group consisting of geographical region, consanguinity, average family size, availability of medical records, and life expectancy.

24. The method of claim 1, wherein the method further comprises:
obtaining a biological sample from each member of said first test population, prior to said identifying step (A); and
determining, for each respective member i of said first test population, a genotype Xᵢ from the biological sample obtained from the respective member of said first test population, prior to said identifying step (A).

25. The method of claim 1, wherein the first test population comprises more than 500 members and the first index founder population comprises less than 500 members.

26. The method of claim 1, wherein the first test population comprises more than 1000 members and the first index founder population comprises less than 1000 members.

27. The method of claim 1, wherein the first test population comprises more than 2500 members and the first index founder population comprises less than 2500 members.

28. The method of claim 1, wherein the one or more chromosomal regions encompasses a dominant or recessive necessity gene.

29. The method of claim 1, wherein the one or more chromosomal regions encompasses a dominant or recessive sufficiency gene.

30. The method of claim 1, the method further comprising:
(D) communicating the identity of the one or more chromosomal regions.

31. The method of claim 1 wherein said first index founder population is Arabic.

32. The method of claim 1, wherein said first index founder population is Indian.

33. The method of claim 1, wherein said first index founder population is African.

34. The method of claim 1, wherein said first index founder population is Indo-Chinese.

35. The method of claim 1, wherein said first index founder population is of Eur-Asian.

36. The method of claim 1, wherein said genotype X comprises a plurality of markers present in the human genome at an average marker density of at least 1 marker per 10 kilobases of human genome.

37. The method of claim 1, wherein said genotype X comprises a plurality of markers present in the human genome at an average marker density of at least 1 marker per 3 kilobases of human genome.

38. The method of claim 1, the method further comprising:
(D) performing an expression analysis of one or more genes within the one or more candidate chromosomal regions in which expression of the one or more genes in members of the first index founder population is correlated with variation in the clinical parameter exhibited by members of the first index founder population.

39. The method of claim 1, further comprising:
(D) identifying a second index founder population in a second test population based upon the genotype X of each member of said second test population, wherein the posterior probability Pr(K|X) for said second index founder population is greater for K=1 than any other integer K, where K is the number of subpopulations in said second index founder population;
(E) measuring said clinical parameter for each respective member of said second index founder population;
(F) performing a quantitative phenotypic analysis between (i) the genotype X of each respective member of said second index founder population and (ii) the clinical parameter thereby identifying one or more candidate chromosomal regions in the human genome that associate with the clinical parameter; and
(G) forming a composite genetic locus associated with the clinical parameter by taking the intersection of the one or more chromosomal regions found in the first index founder population and the one or more chromosomal regions found in the second index founder population.

40. The method of claim 39, wherein the first index founder population is Arabic and the second index founder population is Indian.

41. The method of claim 39, wherein the first index founder population is Arabic, Indian, African, Indo-Chinese or Eur-Asian and the second index founder population is Arabic, Indian, African, Indo-Chinese, or Eur-Asian.

42. The method of claim 1, wherein a variation used in the performing step (C) is a variation in a genotype call of a single nucleotide polymorphism in the genotype X across the members of the first index founder population.

43. A computer program product for use in conjunction with a computer system, the computer program product comprising a user readable storage medium and a computer program mechanism embedded therein, wherein the computer program mechanism is for associating a clinical parameter with one or more candidate chromosomal regions in the human genome, the computer program mechanism comprising:
(A) instructions for identifying an index founder population in a test population based upon the genotype X of each member of said test population, wherein the posterior probability Pr(K|X) for said index founder population is greater for K=1 than any other integer K, wherein K is a number of subpopulations in said index founder population;
(B) instructions for receiving measurements of said clinical parameter for each respective member of said index founder population; and
(C) instructions for performing a quantitative phenotypic analysis between (i) the genotype X of each respective member of said index founder population and (ii) the clinical parameter thereby identifying one or more candidate chromosomal regions in the human genome that associate with the clinical parameter.

44. The computer program product of claim 43, further comprising:
(D) instructions for communicating the identity of the one or more chromosomal regions.

45. A computer system for associating a clinical parameter with one or more candidate chromosomal regions in the human genome, the computer system comprising a processor, and a memory encoding one or more programs coupled to the processor, wherein the one or more programs cause the processor to perform a method comprising:
(A) instructions for identifying an index founder population in a test population based upon the genotype X of each member of said test population, wherein the posterior probability Pr(K|X) for said index founder population is greater for K=1 than any other integer K, wherein K is a number of subpopulations in said index founder population;
(B) instructions for receiving measurements of said clinical parameter for each respective member of said index founder population; and
(C) instructions for performing a quantitative phenotypic analysis between (i) the genotype X of each respective member of said index founder population and (ii) the clinical parameter thereby identifying one or more candidate chromosomal regions in the human genome that associate with the clinical parameter.

46. The computer system of claim 45, wherein the method further comprises:
(D) instructions for communicating the identity of the one or more chromosomal regions.

47. A method of identifying an index founder population comprising:
(A) determining whether the consanguinity rate of a test population is ten percent or greater; and
(B) determining whether the posterior probability Pr(K|X) for the test population is greater for K=1 than any other integer K, where X is a test population member genotype and where K is a number of subpopulations in said test population, wherein
the test population is deemed to be an index founder population when both (i) the determining step (A) determines that the consanguinity rate of a test population is ten percent or greater and (ii) the posterior probability Pr(K|X) for the test population is greater for K=1 than any other positive integer K.

48. The method of claim 47, the method further comprising:
(C) measuring a clinical parameter for each respective member of said index founder population; and
(D) performing a quantitative phenotypic analysis between (i) the genotype X of each respective member of said index founder population and (ii) the clinical parameter thereby identifying one or more candidate chromosomal regions in the human genome that associate with the clinical parameter.

49. The method of claim 48, wherein said quantitative phenotypic analysis is linkage analysis and a chromosomal region in the one or more candidate chromosomal regions is a quantitative trait locus (QTL).

50. The method of claim 48, wherein said quantitative phenotypic analysis is association analysis and a chromosomal region in the one or more candidate chromosomal regions is a QTL.

51. The method of claim 47, wherein said genotype X comprises at least five markers.

52. The method of claim 47, wherein said genotype X comprises at least one hundred markers.

53. The method of claim 47, wherein said genotype X comprises at least one thousand markers.

54. The method of claim 47, wherein said genotype X comprises at least twenty thousand markers.

55. The method of claim 47, wherein said genotype X comprises a haplotype.

56. The method of claim 47, wherein said clinical parameter is an abundance level measurement for a gene in a biological sample obtained from said respective member.

57. The method of claim 56, wherein said abundance level measurement for said gene is determined by measuring an amount of a cellular constituent for said gene in one or more cells in said biological sample.

58. The method of claim 57, wherein the amount of the cellular constituent for said gene comprises an abundance measurement of mRNA transcripts, cDNAs, or cRNAs for mRNA transcribed from the gene, or nucleic acid derived from any of the foregoing.

59. The method of claim 57, wherein the amount of the cellular constituent comprises an abundance of a protein encoded by the gene that are present in or secreted by one or more cells in said biological sample.

60. The method of claim 56, wherein said biological sample is obtained from a single tissue type or a single organ type in said respective member.

61. The method of claim 48, wherein the clinical parameter is absence, presence, or stage of a disease.

62. The method of claim 61, wherein the disease is a complex disease.

63. The method of claim 47, wherein the posterior probability Pr(K|X) for said index founder population for any K less than 6 and greater than 1 is 0.4 or less.

64. The method of claim 47, wherein the posterior probability Pr(K|X) for said index founder population for any K less than 6 and greater than 1 is 0.3 or less.

65. The method of claim 47, the method further comprising, prior to said determining step (A), determining that the average coefficient of inbreeding F_{avg} in the test population is 0.10 or greater.

66. The method of claim 47, the method further comprising, prior to said determining step (A), determining that the average coefficient of inbreeding F_{avg} in the test population is 0.20 or greater.

67. The method of claim 47, the method further comprising, prior to said determining step (A), identifying each member of the test population using at least one criterion selected from the group consisting of geographical region, consanguinity, average family size, availability of medical records, and life expectancy.

68. The method of claim 47, wherein the method further comprises:
obtaining a biological sample from each member of said test population, prior to said determining step (A); and
determining, for each respective member i of said test population, a genotype Xᵢ from the biological sample obtained from the respective member of said test population, prior to said determining step (A).

69. The method of claim 47, wherein the test population comprises more than 500 members and the index founder population comprises less than 500 members.

70. The method of claim 47, wherein the test population comprises more than 1000 members and the index founder population comprises less than 1000 members.

71. The method of claim 47, wherein the test population comprises more than 2500 members and the index founder population comprises less than 2500 members.

72. The method of claim 47, wherein the one or more chromosomal regions encompasses a dominant or recessive necessity gene.

73. The method of claim 47, wherein the one or more chromosomal regions encompasses a dominant or recessive sufficiency gene.

74. The method of claim 47, the method further comprising:
(E) communicating the identity of the one or more chromosomal regions.

75. The method of claim 74, wherein said communicating step (E) comprises communicating the identity of the one or more chromosomal regions to a user, a display, an internal or external component of a computer, a remote computer, or to storage on a computer readable medium.

76. The method of claim 47, wherein said index founder population is Arabic.

77. The method of claim 47, wherein said index founder population is Indian.

78. The method of claim 47, wherein said index founder population is African.

79. The method of claim 47, wherein said index founder population is Indo-Chinese.

80. The method of claim 47, wherein said index founder population is Eur-Asian.

81. The method of claim 47, wherein said population member genotype X comprises a plurality of markers that are present in the human genome at an average marker density of at least 1 marker per 10 kilobases of human genome.

82. The method of claim 47, wherein said population member genotype X comprises a plurality of markers that are present in the human genome at an average marker density of at least 1 marker per 3 kilobases of human genome.

83. The method of claim 48, the method further comprising:
(F) performing an expression analysis of one or more genes within the one or more candidate chromosomal regions in which expression of the one or more genes in members of the index founder population is correlated with variation in the clinical parameter exhibited by members of the index founder population.

84. The method of claim 48, wherein a variation used in the performing step (C) is a variation in a genotype call of a single nucleotide polymorphism in the genotype X across the members of the index founder population.

85. A computer program product for use in conjunction with a computer system, the computer program product comprising a user readable storage medium and a computer program mechanism embedded therein, wherein the computer program mechanism is comprises instructions for carrying out the method of claim 47.

86. A computer program product for use in conjunction with a computer system, the computer program product comprising a user readable storage medium and a computer program mechanism embedded therein, wherein the computer program mechanism is comprises instructions for carrying out the method of claim 48.

87. A computer system comprising a processor, and a memory encoding one or more programs coupled to the processor, wherein the one or more programs cause the processor to perform the method of claim 47.

88. A computer system comprising a processor, and a memory encoding one or more programs coupled to the processor, wherein the one or more programs cause the processor to perform the method of claim 48.
